SEQUENCE LISTING (464 sequences)

SEQ ID NO: 1 - DNA, 21 bp, synthetic construct
  source 1..21: mol_type = other DNA
gagtttcggcggcgtttttcg

SEQ ID NO: 2 - DNA, 22 bp, synthetic construct
  source 1..22: mol_type = other DNA
cgctacgtctaacttcccgcgc

SEQ ID NO: 3 - DNA, 29 bp, synthetic construct
  source 1..29: mol_type = other DNA
ttttcgacgagtaggattgaagaaggaac

SEQ ID NO: 4 - DNA, 25 bp, synthetic construct
  source 1..25: mol_type = other DNA
gcgaatctatctaccgaaacgcgct

SEQ ID NO: 5 - DNA, 31 bp, synthetic construct
  source 1..31: mol_type = other DNA
ttttgatttgtaatatagaggaaagcgtcgt

SEQ ID NO: 6 - DNA, 31 bp, synthetic construct
  source 1..31: mol_type = other DNA
gtataaacgcgtaaataccaaactaaacgaa

SEQ ID NO: 7 - DNA, 25 bp, synthetic construct
  source 1..25: mol_type = other DNA
gtttcgagaaaggagaagggggagc

SEQ ID NO: 8 - DNA, 25 bp, synthetic construct
  source 1..25: mol_type = other DNA
actcccaacgaaaacttcgcaaacg

SEQ ID NO: 9 - DNA, 23 bp, synthetic construct
  source 1..23: mol_type = other DNA
gttttttgtcgggagttattcgt

SEQ ID NO: 10 - DNA, 28 bp, synthetic construct
  source 1..28: mol_type = other DNA
ccaaaaattaaattaaaaacgctacgca

SEQ ID NO: 11 - DNA, 20 bp, synthetic construct
  source 1..20: mol_type = other DNA
gttttaggagggtggggcgt

SEQ ID NO: 12 - DNA, 25 bp, synthetic construct
  source 1..25: mol_type = other DNA
aacacgactattcgaaaaacgcgca

SEQ ID NO: 13 - DNA, 20 bp, synthetic construct
  source 1..20: mol_type = other DNA
ttcgtagtatcgggagtcga

SEQ ID NO: 14 - DNA, 24 bp, synthetic construct
  source 1..24: mol_type = other DNA
gaaataataaaaacgccgcacgct

SEQ ID NO: 15 - DNA, 25 bp, synthetic construct
  source 1..25: mol_type = other DNA
gtcgtagttttcgcgggtggtaagc

SEQ ID NO: 16 - DNA, 25 bp, synthetic construct
  source 1..25: mol_type = other DNA
cgaacgctacctaaactctcccgac

SEQ ID NO: 17 - DNA, 25 bp, synthetic construct
  source 1..25: mol_type = other DNA
ggaatcgcgagttttgggatagtcg

SEQ ID NO: 18 - DNA, 25 bp, synthetic construct
  source 1..25: mol_type = other DNA
aaatacaattacaccctctaccgcc

SEQ ID NO: 19 - DNA, 20 bp, synthetic construct
  source 1..20: mol_type = other DNA
gaggcgttcggtgggatttc

SEQ ID NO: 20 - DNA, 25 bp, synthetic construct
  source 1..25: mol_type = other DNA
ccccgacctataaacctacgacgct

SEQ ID NO: 21 - DNA, 20 bp, synthetic construct
  source 1..20: mol_type = other DNA
gaggaggtagcgggcgtcga

SEQ ID NO: 22 - DNA, 25 bp, synthetic construct
  source 1..25: mol_type = other DNA
cgcgtcgatctaacttacctacgaa

SEQ ID NO: 23 - DNA, 25 bp, synthetic construct
  source 1..25: mol_type = other DNA
ttgcgtttatcgatttcgttttcgt

SEQ ID NO: 24 - DNA, 29 bp, synthetic construct
  source 1..29: mol_type = other DNA
gcactactatcccccgaactactctacgc

SEQ ID NO: 25 - DNA, 24 bp, synthetic construct
  source 1..24: mol_type = other DNA
ttattaggcggggagtcgggtgtc

SEQ ID NO: 26 - DNA, 25 bp, synthetic construct
  source 1..25: mol_type = other DNA
ctcgaatccctaaaaaactcgcgaa

SEQ ID NO: 27 - DNA, 25 bp, synthetic construct
  source 1..25: mol_type = other DNA
aggaaattcggtagcgattatacgg

SEQ ID NO: 28 - DNA, 28 bp, synthetic construct
  source 1..28: mol_type = other DNA
aaacccctacaacctcaccgtacacgat

SEQ ID NO: 29 - DNA, 25 bp, synthetic construct
  source 1..25: mol_type = other DNA
cggagttaataggtacgggaggcgt

SEQ ID NO: 30 - DNA, 22 bp, synthetic construct
  source 1..22: mol_type = other DNA
caaacccccgaactatcgcgaa

SEQ ID NO: 31 - DNA, 22 bp, synthetic construct
  source 1..22: mol_type = other DNA
cgggtatcgcggttaagttggc

SEQ ID NO: 32 - DNA, 25 bp, synthetic construct
  source 1..25: mol_type = other DNA
tatcgtaaaaacccaacccctcgac

SEQ ID NO: 33 - DNA, 23 bp, synthetic construct
  source 1..23: mol_type = other DNA
gggattggtgagattcgggacgt

SEQ ID NO: 34 - DNA, 25 bp, synthetic construct
  source 1..25: mol_type = other DNA
ctccccgaaaccaaaaaacaacgaa

SEQ ID NO: 35 - DNA, 25 bp, synthetic construct
  source 1..25: mol_type = other DNA
gttttaaatcgtattcgtagttcgg

SEQ ID NO: 36 - DNA, 25 bp, synthetic construct
  source 1..25: mol_type = other DNA
acgaacgaaaactttcctaaacgaa

SEQ ID NO: 37 - DNA, 25 bp, synthetic construct
  source 1..25: mol_type = other DNA
gcgtttttttatcgttttagggcgt

SEQ ID NO: 38 - DNA, 24 bp, synthetic construct
  source 1..24: mol_type = other DNA
accgacactaccaacctctccgaa

SEQ ID NO: 39 - DNA, 22 bp, synthetic construct
  source 1..22: mol_type = other DNA
tgcggggatttttagcggaagc

SEQ ID NO: 40 - DNA, 28 bp, synthetic construct
  source 1..28: mol_type = other DNA
ccgacgaactatccgactatcactcgtt

SEQ ID NO: 41 - DNA, 24 bp, synthetic construct
  source 1..24: mol_type = other DNA
gtagtaggtggagggggcgagttc

SEQ ID NO: 42 - DNA, 24 bp, synthetic construct
  source 1..24: mol_type = other DNA
ctctcgaaaaccgcaaaatcctcg

SEQ ID NO: 43 - DNA, 27 bp, synthetic construct
  source 1..27: mol_type = other DNA
ggtaatattgcgatatttcgtagacgt

SEQ ID NO: 44 - DNA, 25 bp, synthetic construct
  source 1..25: mol_type = other DNA
aacaatcaaataatcgaacgcacgc

SEQ ID NO: 45 - DNA, 23 bp, synthetic construct
  source 1..23: mol_type = other DNA
gtcgtttttcgttacgaagcggc

SEQ ID NO: 46 - DNA, 25 bp, synthetic construct
  source 1..25: mol_type = other DNA
aaaactaaataaatctatcctcgat

SEQ ID NO: 47 - DNA, 24 bp, synthetic construct
  source 1..24: mol_type = other DNA
gcgtcggcggtttttagtaaaagc

SEQ ID NO: 48 - DNA, 25 bp, synthetic construct
  source 1..25: mol_type = other DNA
aacgaatctcattaaatctcccgtc

SEQ ID NO: 49 - DNA, 18 bp, synthetic construct
  source 1..18: mol_type = other DNA
gattttcgggagcggcga

SEQ ID NO: 50 - DNA, 18 bp, synthetic construct
  source 1..18: mol_type = other DNA
cttccccgcaacgaaccg

SEQ ID NO: 51 - DNA, 25 bp, synthetic construct
  source 1..25: mol_type = other DNA
ttcgttggtatattcgcgtaggtgc

SEQ ID NO: 52 - DNA, 26 bp, synthetic construct
  source 1..26: mol_type = other DNA
cgaataccgaaatctataaccccgaa

SEQ ID NO: 53 - DNA, 23 bp, synthetic construct
  source 1..23: mol_type = other DNA
attatgattacgatggttgacgg

SEQ ID NO: 54 - DNA, 25 bp, synthetic construct
  source 1..25: mol_type = other DNA
ctccataaaaacgaatttaaacgaa

SEQ ID NO: 55 - DNA, 25 bp, synthetic construct
  source 1..25: mol_type = other DNA
tttggttatagaacgtagaggtcgt

SEQ ID NO: 56 - DNA, 22 bp, synthetic construct
  source 1..22: mol_type = other DNA
atcgaaccaccaaaccaaacgc

SEQ ID NO: 57 - DNA, 22 bp, synthetic construct
  source 1..22: mol_type = other DNA
gggaagtttagtaggtgagcgt

SEQ ID NO: 58 - DNA, 25 bp, synthetic construct
  source 1..25: mol_type = other DNA
actaaaaacgtttccgtcgaacgca

SEQ ID NO: 59 - DNA, 25 bp, synthetic construct
  source 1..25: mol_type = other DNA
gttggtaggagtagggttggttcga

SEQ ID NO: 60 - DNA, 24 bp, synthetic construct
  source 1..24: mol_type = other DNA
atcgcaatcgtaacccgtaaacgc

SEQ ID NO: 61 - DNA, 25 bp, synthetic construct
  source 1..25: mol_type = other DNA
attcgtacggttttttcgttttcgt

SEQ ID NO: 62 - DNA, 23 bp, synthetic construct
  source 1..23: mol_type = other DNA
gaccaactacttccgctcgacgc

SEQ ID NO: 63 - DNA, 20 bp, synthetic construct
  source 1..20: mol_type = other DNA
cggatttagcggtcgagacg

SEQ ID NO: 64 - DNA, 23 bp, synthetic construct
  source 1..23: mol_type = other DNA
tttaaaacgtttctcgcgacgcc

SEQ ID NO: 65 - DNA, 24 bp, synthetic construct
  source 1..24: mol_type = other DNA
tcgttaggcgatgataattagcga

SEQ ID NO: 66 - DNA, 25 bp, synthetic construct
  source 1..25: mol_type = other DNA
taaaaaaaccataaaccctaacgac

SEQ ID NO: 67 - DNA, 25 bp, synthetic construct
  source 1..25: mol_type = other DNA
gttggagaagacgattcgttcggac

SEQ ID NO: 68 - DNA, 23 bp, synthetic construct
  source 1..23: mol_type = other DNA
ccaaaacctcactcctcaaccgc

SEQ ID NO: 69 - DNA, 19 bp, synthetic construct
  source 1..19: mol_type = other DNA
cgcgatgcgcgttttgaac

SEQ ID NO: 70 - DNA, 27 bp, synthetic construct
  source 1..27: mol_type = other DNA
gacgcgactaacttccaacctaacgaa

SEQ ID NO: 71 - DNA, 22 bp, synthetic construct
  source 1..22: mol_type = other DNA
ttttcgtggttgtcgtcgttgc

SEQ ID NO: 72 - DNA, 27 bp, synthetic construct
  source 1..27: mol_type = other DNA
gccgcgctctacactaaacatattcgc

SEQ ID NO: 73 - DNA, 23 bp, synthetic construct
  source 1..23: mol_type = other DNA
cggggaagtgggagtttttagcg

SEQ ID NO: 74 - DNA, 25 bp, synthetic construct
  source 1..25: mol_type = other DNA
aaaaaaactaaatcaaaaccgcgac

SEQ ID NO: 75 - DNA, 24 bp, synthetic construct
  source 1..24: mol_type = other DNA
gcgagttcgcgttgtttacgtttc

SEQ ID NO: 76 - DNA, 26 bp, synthetic construct
  source 1..26: mol_type = other DNA
accgacgctacctataactccacgct

SEQ ID NO: 77 - DNA, 25 bp, synthetic construct
  source 1..25: mol_type = other DNA
ttaggtttgtttattaattttacgt

SEQ ID NO: 78 - DNA, 18 bp, synthetic construct
  source 1..18: mol_type = other DNA
tctacaaaacgccgcgac

SEQ ID NO: 79 - DNA, 23 bp, synthetic construct
  source 1..23: mol_type = other DNA
gttaattcgagagcgcgaggcgt

SEQ ID NO: 80 - DNA, 28 bp, synthetic construct
  source 1..28: mol_type = other DNA
gaccaaaaaaaataaaaaatcccgcgac

SEQ ID NO: 81 - DNA, 27 bp, synthetic construct
  source 1..27: mol_type = other DNA
taaagaaatagaaagcgggcgatacgt

SEQ ID NO: 82 - DNA, 24 bp, synthetic construct
  source 1..24: mol_type = other DNA
cgaactaaaaaaaccgccaacccg

SEQ ID NO: 83 - DNA, 20 bp, synthetic construct
  source 1..20: mol_type = other DNA
gggttttgcggttaatggcg

SEQ ID NO: 84 - DNA, 25 bp, synthetic construct
  source 1..25: mol_type = other DNA
aataacaaacgcgtcccgaaaacga

SEQ ID NO: 85 - DNA, 32 bp, synthetic construct
  source 1..32: mol_type = other DNA
ttagttttttttggtttttatttgaatttcga

SEQ ID NO: 86 - DNA, 26 bp, synthetic construct
  source 1..26: mol_type = other DNA
aacttttccaccgattctcaattccg

SEQ ID NO: 87 - DNA, 25 bp, synthetic construct
  source 1..25: mol_type = other DNA
atttaaattttcggcgtttcgtcgt

SEQ ID NO: 88 - DNA, 27 bp, synthetic construct
  source 1..27: mol_type = other DNA
acactccaaatcgacctttacaatcgc

SEQ ID NO: 89 - DNA, 25 bp, synthetic construct
  source 1..25: mol_type = other DNA
agtttggttcgtttagcgattgcgt

SEQ ID NO: 90 - DNA, 25 bp, synthetic construct
  source 1..25: mol_type = other DNA
aacgcgactaaaaccaatttccgca

SEQ ID NO: 91 - DNA, 25 bp, synthetic construct
  source 1..25: mol_type = other DNA
tttatttgtttttatcgttcgtcgg

SEQ ID NO: 92 - DNA, 24 bp, synthetic construct
  source 1..24: mol_type = other DNA
aaatatatacccgatttccccgtt

SEQ ID NO: 93 - DNA, 25 bp, synthetic construct
  source 1..25: mol_type = other DNA
taatcggcgtcgagagagatatcgt

SEQ ID NO: 94 - DNA, 22 bp, synthetic construct
  source 1..22: mol_type = other DNA
ccgtcaaccaatcgaaaacgaa

SEQ ID NO: 95 - DNA, 25 bp, synthetic construct
  source 1..25: mol_type = other DNA
tcgtttatttcgttttttttgtcga

SEQ ID NO: 96 - DNA, 25 bp, synthetic construct
  source 1..25: mol_type = other DNA
aaccaacctaaaatctacactcgca

SEQ ID NO: 97 - DNA, 20 bp, synthetic construct
  source 1..20: mol_type = other DNA
gggtcgtaggggtttatcgc

SEQ ID NO: 98 - DNA, 26 bp, synthetic construct
  source 1..26: mol_type = other DNA
catacttatccgaacgtctaaacgtc

SEQ ID NO: 99 - DNA, 23 bp, synthetic construct
  source 1..23: mol_type = other DNA
ggttttagcgatgaatcggacgt

SEQ ID NO: 100 - DNA, 25 bp, synthetic construct
  source 1..25: mol_type = other DNA
cacgatcttaaaaaaacaacgcgac

SEQ ID NO: 101 - DNA, 25 bp, synthetic construct
  source 1..25: mol_type = other DNA
cgtatttttaggtttagttcggcgt

SEQ ID NO: 102 - DNA, 25 bp, synthetic construct
  source 1..25: mol_type = other DNA
acactattacccgcgaaaaaacgat

SEQ ID NO: 103 - DNA, 27 bp, synthetic construct
  source 1..27: mol_type = other DNA
gagtttgtttgggggttggtcgtattc

SEQ ID NO: 104 - DNA, 31 bp, synthetic construct
  source 1..31: mol_type = other DNA
ccaaatataacgtttaactctttaccacgaa

SEQ ID NO: 105 - DNA, 25 bp, synthetic construct
  source 1..25: mol_type = other DNA
ttttttttgattcggattttttcgg

SEQ ID NO: 106 - DNA, 25 bp, synthetic construct
  source 1..25: mol_type = other DNA
ctaataaacgccgccgtattcgacg

SEQ ID NO: 107 - DNA, 25 bp, synthetic construct
  source 1..25: mol_type = other DNA
ttttcgcgttgtttttatttatcgt

SEQ ID NO: 108 - DNA, 25 bp, synthetic construct
  source 1..25: mol_type = other DNA
tacacaaccacccaactactccgcg

SEQ ID NO: 109 - DNA, 25 bp, synthetic construct
  source 1..25: mol_type = other DNA
tgttgttgggtaaaggttagtacgt

SEQ ID NO: 110 - DNA, 19 bp, synthetic construct
  source 1..19: mol_type = other DNA
cgaaaacccaactcccgaa

SEQ ID NO: 111 - DNA, 23 bp, synthetic construct
  source 1..23: mol_type = other DNA
tttttgcggtcgtttttcggagc

SEQ ID NO: 112 - DNA, 25 bp, synthetic construct
  source 1..25: mol_type = other DNA
cttaccaactaacccccgcctaccg

SEQ ID NO: 113 - DNA, 24 bp, synthetic construct
  source 1..24: mol_type = other DNA
cgttttagtagggattgggggcga

SEQ ID NO: 114 - DNA, 25 bp, synthetic construct
  source 1..25: mol_type = other DNA
cccgaaaaccaaaataaaatccgca

SEQ ID NO: 115 - DNA, 23 bp, synthetic construct
  source 1..23: mol_type = other DNA
cggaatagcgcggtcgttttttc

SEQ ID NO: 116 - DNA, 24 bp, synthetic construct
  source 1..24: mol_type = other DNA
tttaaccgtaacgctcgcctcgac

SEQ ID NO: 117 - DNA, 25 bp, synthetic construct
  source 1..25: mol_type = other DNA
gtcggttgtgtttagagcgtagcgt

SEQ ID NO: 118 - DNA, 21 bp, synthetic construct
  source 1..21: mol_type = other DNA
aaaaaaaaccccgacgacgaa

SEQ ID NO: 119 - DNA, 24 bp, synthetic construct
  source 1..24: mol_type = other DNA
gttgcgattgtttgtattttgcgg

SEQ ID NO: 120 - DNA, 25 bp, synthetic construct
  source 1..25: mol_type = other DNA
ataataacaaaaaacccctcccgac

SEQ ID NO: 121 - DNA, 25 bp, synthetic construct
  source 1..25: mol_type = other DNA
agtttcgttagggaagggttgcgtc

SEQ ID NO: 122 - DNA, 25 bp, synthetic construct
  source 1..25: mol_type = other DNA
caactaaaactctaccgcgctcgat

SEQ ID NO: 123 - DNA, 25 bp, synthetic construct
  source 1..25: mol_type = other DNA
aggaagggtttcgagtttagtgcgg

SEQ ID NO: 124 - DNA, 25 bp, synthetic construct
  source 1..25: mol_type = other DNA
aaaaaaatcaacgcgtcctcctcgc

SEQ ID NO: 125 - DNA, 25 bp, synthetic construct
  source 1..25: mol_type = other DNA
tttcgatttcgtttttaaatttcgt

SEQ ID NO: 126 - DNA, 25 bp, synthetic construct
  source 1..25: mol_type = other DNA
aaactaaacgacctaaccctacgta

SEQ ID NO: 127 - DNA, 25 bp, synthetic construct
  source 1..25: mol_type = other DNA
aagtttacgcgcgagtttgatcgtc

SEQ ID NO: 128 - DNA, 22 bp, synthetic construct
  source 1..22: mol_type = other DNA
cgaaacgacttctctccccgca

SEQ ID NO: 129 - DNA, 25 bp, synthetic construct
  source 1..25: mol_type = other DNA
tttagttcgcggaagttaggttcgg

SEQ ID NO: 130 - DNA, 24 bp, synthetic construct
  source 1..24: mol_type = other DNA
gaaaacacaataaaccccgccgtc

SEQ ID NO: 131 - DNA, 25 bp, synthetic construct
  source 1..25: mol_type = other DNA
gttagattgtaggagggattagcgg

SEQ ID NO: 132 - DNA, 25 bp, synthetic construct
  source 1..25: mol_type = other DNA
aaaaaacgactaaaaaattcacgcc

SEQ ID NO: 133 - DNA, 25 bp, synthetic construct
  source 1..25: mol_type = other DNA
tttggagtttgggggatcgatagtc

SEQ ID NO: 134 - DNA, 25 bp, synthetic construct
  source 1..25: mol_type = other DNA
cgacgaaactaaaaccgcgtacgta

SEQ ID NO: 135 - DNA, 25 bp, synthetic construct
  source 1..25: mol_type = other DNA
tttggagtttgggggatcgatagtc

SEQ ID NO: 136 - DNA, 25 bp, synthetic construct
  source 1..25: mol_type = other DNA
cgacgaaactaaaaccgcgtacgta

SEQ ID NO: 137 - DNA, 25 bp, synthetic construct
  source 1..25: mol_type = other DNA
attatattgggggcgttaggttcgg

SEQ ID NO: 138 - DNA, 25 bp, synthetic construct
  source 1..25: mol_type = other DNA
aacaaacaattcgcacgtaaacgaa

SEQ ID NO: 139 - DNA, 31 bp, synthetic construct
  source 1..31: mol_type = other DNA
gggcggtttacgtggatttttatagattttc

SEQ ID NO: 140 - DNA, 25 bp, synthetic construct
  source 1..25: mol_type = other DNA
gcgtctcgaaccgtaccctaacgta

SEQ ID NO: 141 - DNA, 24 bp, synthetic construct
  source 1..24: mol_type = other DNA
cgtcgttgttgattatgatcgcgg

SEQ ID NO: 142 - DNA, 24 bp, synthetic construct
  source 1..24: mol_type = other DNA
cgcttcctaacaaccttcctcgaa

SEQ ID NO: 143 - DNA, 25 bp, synthetic construct
  source 1..25: mol_type = other DNA
ttaacggtattttttgttttttcgt

SEQ ID NO: 144 - DNA, 22 bp, synthetic construct
  source 1..22: mol_type = other DNA
aaaaaaaactcgtccccgcgct

SEQ ID NO: 145 - DNA, 28 bp, synthetic construct
  source 1..28: mol_type = other DNA
tcggttagttcgaggtaggaagttttgc

SEQ ID NO: 146 - DNA, 30 bp, synthetic construct
  source 1..30: mol_type = other DNA
tattaaccgaaaaacgaaaaccaaatccga

SEQ ID NO: 147 - DNA, 25 bp, synthetic construct
  source 1..25: mol_type = other DNA
agttttgttgttttgggtaggtcgg

SEQ ID NO: 148 - DNA, 25 bp, synthetic construct
  source 1..25: mol_type = other DNA
aaaaactaaaaacctttctctcgac

SEQ ID NO: 149 - DNA, 28 bp, synthetic construct
  source 1..28: mol_type = other DNA
gcgttgagagtgacggatatttttcgtc

SEQ ID NO: 150 - DNA, 25 bp, synthetic construct
  source 1..25: mol_type = other DNA
actacctaaactccgaacacgcccg

SEQ ID NO: 151 - DNA, 25 bp, synthetic construct
  source 1..25: mol_type = other DNA
ttagcgtatcgggaattagggggac

SEQ ID NO: 152 - DNA, 22 bp, synthetic construct
  source 1..22: mol_type = other DNA
gaaaacgaaaaaacgacgcgca

SEQ ID NO: 153 - DNA, 25 bp, synthetic construct
  source 1..25: mol_type = other DNA
tcgttttttaggtggggaagaagcg

SEQ ID NO: 154 - DNA, 26 bp, synthetic construct
  source 1..26: mol_type = other DNA
gaaccgtatttaaaaccaatccccgc

SEQ ID NO: 155 - DNA, 23 bp, synthetic construct
  source 1..23: mol_type = other DNA
aattggggttcggggttcggtac

SEQ ID NO: 156 - DNA, 25 bp, synthetic construct
  source 1..25: mol_type = other DNA
ttacccctacccaaaaaaatacgct

SEQ ID NO: 157 - DNA, 22 bp, synthetic construct
  source 1..22: mol_type = other DNA
ggggttagagtttcgcgttcgc

SEQ ID NO: 158 - DNA, 28 bp, synthetic construct
  source 1..28: mol_type = other DNA
cgcgtctcccgtcctatctatatacgtc

SEQ ID NO: 159 - DNA, 27 bp, synthetic construct
  source 1..27: mol_type = other DNA
taggaattttttaaattcgttttacgg

SEQ ID NO: 160 - DNA, 26 bp, synthetic construct
  source 1..26: mol_type = other DNA
cacaaaaactcgatacaattaccgtt

SEQ ID NO: 161 - DNA, 25 bp, synthetic construct
  source 1..25: mol_type = other DNA
tattttatagtcgcgttaaaagcgt

SEQ ID NO: 162 - DNA, 25 bp, synthetic construct
  source 1..25: mol_type = other DNA
gtcgataaaaaacctacgcgacgaa

SEQ ID NO: 163 - DNA, 26 bp, synthetic construct
  source 1..26: mol_type = other DNA
gatttagtttttcgggtttatagcgg

SEQ ID NO: 164 - DNA, 25 bp, synthetic construct
  source 1..25: mol_type = other DNA
tattaaaaacgaccaaacctccgca

SEQ ID NO: 165 - DNA, 26 bp, synthetic construct
  source 1..26: mol_type = other DNA
tggttgtaggcgttttgttggagttc

SEQ ID NO: 166 - DNA, 25 bp, synthetic construct
  source 1..25: mol_type = other DNA
aaaaacgaccctaaccaccctcgtt

SEQ ID NO: 167 - DNA, 25 bp, synthetic construct
  source 1..25: mol_type = other DNA
ttttgcgtagttgggtagggttcgg

SEQ ID NO: 168 - DNA, 23 bp, synthetic construct
  source 1..23: mol_type = other DNA
cccgcattcccgaaaaaaacgat

SEQ ID NO: 169 - DNA, 25 bp, synthetic construct
  source 1..25: mol_type = other DNA
ttagggtttttttcgaggagttcga

SEQ ID NO: 170 - DNA, 24 bp, synthetic construct
  source 1..24: mol_type = other DNA
atcccccgtacgaaactaaacgcg

SEQ ID NO: 171 - DNA, 23 bp, synthetic construct
  source 1..23: mol_type = other DNA
gcgttcgtattttcgggagaggc

SEQ ID NO: 172 - DNA, 25 bp, synthetic construct
  source 1..25: mol_type = other DNA
tctacgtaactaaacaaaacccgaa

SEQ ID NO: 173 - DNA, 33 bp, synthetic construct
  source 1..33: mol_type = other DNA
cgttttgtgttttataaaaagaaagattttcgg

SEQ ID NO: 174 - DNA, 21 bp, synthetic construct
  source 1..21: mol_type = other DNA
aaaaccccaaaaacgcccgat

SEQ ID NO: 175 - DNA, 27 bp, synthetic construct
  source 1..27: mol_type = other DNA
ggggcgtatatattagttatcgagcga

SEQ ID NO: 176 - DNA, 25 bp, synthetic construct
  source 1..25: mol_type = other DNA
aaaaaaaaccctaaaaaccgccgaa

SEQ ID NO: 177 - DNA, 26 bp, synthetic construct
  source 1..26: mol_type = other DNA
ttcgtttaatgagaaggggttagcgg

SEQ ID NO: 178 - DNA, 31 bp, synthetic construct
  source 1..31: mol_type = other DNA
taaaacaaactaaaaaccttaacgcgacgct

SEQ ID NO: 179 - DNA, 20 bp, synthetic construct
  source 1..20: mol_type = other DNA
ggggagggagttttttttac

SEQ ID NO: 180 - DNA, 25 bp, synthetic construct
  source 1..25: mol_type = other DNA
gtacgcgaactcgccaaacactacg

SEQ ID NO: 181 - DNA, 25 bp, synthetic construct
  source 1..25: mol_type = other DNA
gtagggttggtagtcgtttttacgt

SEQ ID NO: 182 - DNA, 28 bp, synthetic construct
  source 1..28: mol_type = other DNA
aacccatctaattacaaaatacctcgat

SEQ ID NO: 183 - DNA, 26 bp, synthetic construct
  source 1..26: mol_type = other DNA
ggttttataggggaaattattttcgt

SEQ ID NO: 184 - DNA, 25 bp, synthetic construct
  source 1..25: mol_type = other DNA
aaaacctcgtctttataacatcgaa

SEQ ID NO: 185 - DNA, 27 bp, synthetic construct
  source 1..27: mol_type = other DNA
taggatatttcgatgttataaagacga

SEQ ID NO: 186 - DNA, 25 bp, synthetic construct
  source 1..25: mol_type = other DNA
aacaaaactaacaaccgcctccacg

SEQ ID NO: 187 - DNA, 27 bp, synthetic construct
  source 1..27: mol_type = other DNA
tttggagttatcggaaggcgaaagtac

SEQ ID NO: 188 - DNA, 24 bp, synthetic construct
  source 1..24: mol_type = other DNA
gcacgccgaaaaaataaaaacgaa

SEQ ID NO: 189 - DNA, 25 bp, synthetic construct
  source 1..25: mol_type = other DNA
ttttcgatatcgatatcgaaggcgt

SEQ ID NO: 190 - DNA, 25 bp, synthetic construct
  source 1..25: mol_type = other DNA
ataacttaaaaccctaaattccgcc

SEQ ID NO: 191 - DNA, 26 bp, synthetic construct
  source 1..26: mol_type = other DNA
gcgggtagtaggaagattagtagcgg

SEQ ID NO: 192 - DNA, 22 bp, synthetic construct
  source 1..22: mol_type = other DNA
ccgacttccgtacgaaaccgta

SEQ ID NO: 193 - DNA, 25 bp, synthetic construct
  source 1..25: mol_type = other DNA
taatagaggtttgcgttggaatcga

SEQ ID NO: 194 - DNA, 25 bp, synthetic construct
  source 1..25: mol_type = other DNA
aacgcaccctaaacaaaaccacgac

SEQ ID NO: 195 - DNA, 26 bp, synthetic construct
  source 1..26: mol_type = other DNA
tgaagagttgttagttcgtttagcgt

SEQ ID NO: 196 - DNA, 23 bp, synthetic construct
  source 1..23: mol_type = other DNA
gccaaaaattcgattccaacgca

SEQ ID NO: 197 - DNA, 25 bp, synthetic construct
  source 1..25: mol_type = other DNA
tagtggtaggtatagttggtagcgg

SEQ ID NO: 198 - DNA, 25 bp, synthetic construct
  source 1..25: mol_type = other DNA
atcaaaactcccctcctcgaaaacg

SEQ ID NO: 199 - DNA, 19 bp, synthetic construct
  source 1..19: mol_type = other DNA
gtttttaagcggcggtcgt

SEQ ID NO: 200 - DNA, 19 bp, synthetic construct
  source 1..19: mol_type = other DNA
aaaaaaaatcccgttcgct

SEQ ID NO: 201 - DNA, 24 bp, synthetic construct
  source 1..24: mol_type = other DNA
gcgcgcgtttattagatgaagtcg

SEQ ID NO: 202 - DNA, 27 bp, synthetic construct
  source 1..27: mol_type = other DNA
aaaatcaaaaaccacaaattcaccgcc

SEQ ID NO: 203 - DNA, 25 bp, synthetic construct
  source 1..25: mol_type = other DNA
cggggagaggaggggtaggatttac

SEQ ID NO: 204 - DNA, 25 bp, synthetic construct
  source 1..25: mol_type = other DNA
caacttaaacaccacttcctccgaa

SEQ ID NO: 205 - DNA, 20 bp, synthetic construct
  source 1..20: mol_type = other DNA
tgttttttttgttcgggcgg

SEQ ID NO: 206 - DNA, 28 bp, synthetic construct
  source 1..28: mol_type = other DNA
aaataactaactcctactctcgcccgct

SEQ ID NO: 207 - DNA, 29 bp, synthetic construct
  source 1..29: mol_type = other DNA
atagttttttaattttcgcgtttcgtcga

SEQ ID NO: 208 - DNA, 24 bp, synthetic construct
  source 1..24: mol_type = other DNA
aaaaacaactccaacccacaccgc

SEQ ID NO: 209 - DNA, 25 bp, synthetic construct
  source 1..25: mol_type = other DNA
gcgggaggagtaggttaattttcga

SEQ ID NO: 210 - DNA, 25 bp, synthetic construct
  source 1..25: mol_type = other DNA
aaaaacaaaatacgcgaaacgcacg

SEQ ID NO: 211 - DNA, 26 bp, synthetic construct
  source 1..26: mol_type = other DNA
cgagaaggttttgtcgtagacgtcgt

SEQ ID NO: 212 - DNA, 22 bp, synthetic construct
  source 1..22: mol_type = other DNA
tacgtatccatacccgcgctcg

SEQ ID NO: 213 - DNA, 25 bp, synthetic construct
  source 1..25: mol_type = other DNA
tttgtttgtataataggggttgcgg

SEQ ID NO: 214 - DNA, 25 bp, synthetic construct
  source 1..25: mol_type = other DNA
cgcctaactaccgaaaaataccgaa

SEQ ID NO: 215 - DNA, 30 bp, synthetic construct
  source 1..30: mol_type = other DNA
ggtggggttatttttttatggagtcgattc

SEQ ID NO: 216 - DNA, 24 bp, synthetic construct
  source 1..24: mol_type = other DNA
cgaaccaaacctacgattcccgaa

SEQ ID NO: 217 - DNA, 27 bp, synthetic construct
  source 1..27: mol_type = other DNA
gggagaagagaatggttttttgtcgtc

SEQ ID NO: 218 - DNA, 27 bp, synthetic construct
  source 1..27: mol_type = other DNA
tcttatactcaaccccgacctaccgac

SEQ ID NO: 219 - DNA, 31 bp, synthetic construct
  source 1..31: mol_type = other DNA
gttttattcggggttttagcgttatttacgg

SEQ ID NO: 220 - DNA, 24 bp, synthetic construct
  source 1..24: mol_type = other DNA
aaaaaaccgcgttactcaacgcgc

SEQ ID NO: 221 - DNA, 24 bp, synthetic construct
  source 1..24: mol_type = other DNA
gtttagagcggaggtagcggttgc

SEQ ID NO: 222 - DNA, 26 bp, synthetic construct
  source 1..26: mol_type = other DNA
cgcctattcttaaacctaaacccgtc

SEQ ID NO: 223 - DNA, 27 bp, synthetic construct
  source 1..27: mol_type = other DNA
cgtagaggattataaagatttgtacga

SEQ ID NO: 224 - DNA, 30 bp, synthetic construct
  source 1..30: mol_type = other DNA
tactataactactacgataacgacgacgac

SEQ ID NO: 225 - DNA, 28 bp, synthetic construct
  source 1..28: mol_type = other DNA
agatttcggtttttgtttcgattttcgt

SEQ ID NO: 226 - DNA, 25 bp, synthetic construct
  source 1..25: mol_type = other DNA
attaatactaacttacgaaaccgcc

SEQ ID NO: 227 - DNA, 25 bp, synthetic construct
  source 1..25: mol_type = other DNA
attatttttgagcgtgaaaaatcgt

SEQ ID NO: 228 - DNA, 26 bp, synthetic construct
  source 1..26: mol_type = other DNA
aaatttctctccaattaaattccgta

SEQ ID NO: 229 - DNA, 22 bp, synthetic construct
  source 1..22: mol_type = other DNA
gtgggtttgtcgtcggatttcg

SEQ ID NO: 230 - DNA, 25 bp, synthetic construct
  source 1..25: mol_type = other DNA
aaataaccgcgtcatccgattcgtt

SEQ ID NO: 231 - DNA, 34 bp, synthetic construct
  source 1..34: mol_type = other DNA
tggatgttttatattaatttttagttgtataacg

SEQ ID NO: 232 - DNA, 31 bp, synthetic construct
  source 1..31: mol_type = other DNA
gtactttttctctcacgaaaaatattcccgc

SEQ ID NO: 233 - DNA, 25 bp, synthetic construct
  source 1..25: mol_type = other DNA
tgcgtggaataaattttatatacgt

SEQ ID NO: 234 - DNA, 25 bp, synthetic construct
  source 1..25: mol_type = other DNA
gctcaacacacgaaaaaccctcgaa

SEQ ID NO: 235 - DNA, 25 bp, synthetic construct
  source 1..25: mol_type = other DNA
cggtgcggggttttaataaaggatc

SEQ ID NO: 236 - DNA, 25 bp, synthetic construct
  source 1..25: mol_type = other DNA
tccacgcaaaaacaaaaaacgcgta

SEQ ID NO: 237 - DNA, 27 bp, synthetic construct
  source 1..27: mol_type = other DNA
ggcgtagttatgatttcgttttttcgt

SEQ ID NO: 238 - DNA, 26 bp, synthetic construct
  source 1..26: mol_type = other DNA
atcctttcgaccctacgtacctcgat

SEQ ID NO: 239 - DNA, 28 bp, synthetic construct
  source 1..28: mol_type = other DNA
tttacgattattgttttagataatacgg

SEQ ID NO: 240 - DNA, 19 bp, synthetic construct
  source 1..19: mol_type = other DNA
gaacccgacgaacttcgaa

SEQ ID NO: 241 - DNA, 21 bp, synthetic construct
  source 1..21: mol_type = other DNA
gggaaatcgcgtagtttgggc

SEQ ID NO: 242 - DNA, 25 bp, synthetic construct
  source 1..25: mol_type = other DNA
aaaacgacgaaaaaacgaaaacgac

SEQ ID NO: 243 - DNA, 26 bp, synthetic construct
  source 1..26: mol_type = other DNA
agttatcgcgatcggtttgggttaac

SEQ ID NO: 244 - DNA, 28 bp, synthetic construct
  source 1..28: mol_type = other DNA
aaacgattacctctttcgttcgttcgtt

SEQ ID NO: 245 - DNA, 24 bp, synthetic construct
  source 1..24: mol_type = other DNA
cggcggtttatttgaagagggttc

SEQ ID NO: 246 - DNA, 25 bp, synthetic construct
  source 1..25: mol_type = other DNA
cgacaaatcaaaaatctacaacgct

SEQ ID NO: 247 - DNA, 25 bp, synthetic construct
  source 1..25: mol_type = other DNA
ttttaacgttagttacgagttgcgg

SEQ ID NO: 248 - DNA, 21 bp, synthetic construct
  source 1..21: mol_type = other DNA
cgaacaaaccaaacaaccgaa

SEQ ID NO: 249 - DNA, 19 bp, synthetic construct
  source 1..19: mol_type = other DNA
gtagattaggcgggggcga

SEQ ID NO: 250 - DNA, 34 bp, synthetic construct
  source 1..34: mol_type = other DNA
gaacaaaaacataaactaatacaaatatctcccg

SEQ ID NO: 251 - DNA, 24 bp, synthetic construct
  source 1..24: mol_type = other DNA
ggagggagagtttttcgcggattc

SEQ ID NO: 252 - DNA, 25 bp, synthetic construct
  source 1..25: mol_type = other DNA
ctaaacccctcaaaccctaaccgat

SEQ ID NO: 253 - DNA, 20 bp, synthetic construct
  source 1..20: mol_type = other DNA
tgttttcggatacggcgagc

SEQ ID NO: 254 - DNA, 23 bp, synthetic construct
  source 1..23: mol_type = other DNA
acgaacgaactatacgcgacgct

SEQ ID NO: 255 - DNA, 21 bp, synthetic construct
  source 1..21: mol_type = other DNA
tggtaatcgggtttttcgacg

SEQ ID NO: 256 - DNA, 22 bp, synthetic construct
  source 1..22: mol_type = other DNA
ccgcgaatctatctaccgaaac

SEQ ID NO: 257 - DNA, 19 bp, synthetic construct
  source 1..19: mol_type = other DNA
gtgttaggttgggcggaag

SEQ ID NO: 258 - DNA, 22 bp, synthetic construct
  source 1..22: mol_type = other DNA
gttacccgcttacgaaaaacga

SEQ ID NO: 259 - DNA, 24 bp, synthetic construct
  source 1..24: mol_type = other DNA
ttcgttttttgtcgggagttattc

SEQ ID NO: 260 - DNA, 28 bp, synthetic construct
  source 1..28: mol_type = other DNA
tccaaaaattaaattaaaaacgctacgc

SEQ ID NO: 261 - DNA, 21 bp, synthetic construct
  source 1..21: mol_type = other DNA
ataatacggttgttcgggagg

SEQ ID NO: 262 - DNA, 25 bp, synthetic construct
  source 1..25: mol_type = other DNA
gtaaatataaactacgcgacgcgta

SEQ ID NO: 263 - DNA, 21 bp, synthetic construct
  source 1..21: mol_type = other DNA
gagagtttaggtagcgttcgg

SEQ ID NO: 264 - DNA, 28 bp, synthetic construct
  source 1..28: mol_type = other DNA
cctaacgctactaacaacattataacga

SEQ ID NO: 265 - DNA, 19 bp, synthetic construct
  source 1..19: mol_type = other DNA
cgcggttttggagcgttag

SEQ ID NO: 266 - DNA, 20 bp, synthetic construct
  source 1..20: mol_type = other DNA
ccgaaacgaccgaaaacgac

SEQ ID NO: 267 - DNA, 20 bp, synthetic construct
  source 1..20: mol_type = other DNA
cggttcgttgcgtttatcga

SEQ ID NO: 268 - DNA, 20 bp, synthetic construct
  source 1..20: mol_type = other DNA
cccccgaactactctacgcg

SEQ ID NO: 269 - DNA, 28 bp, synthetic construct
  source 1..28: mol_type = other DNA
gattatattcggattttgtttatcgcgt

SEQ ID NO: 270 - DNA, 18 bp, synthetic construct
  source 1..18: mol_type = other DNA
gactcttcctacccgcga

SEQ ID NO: 271 - DNA, 19 bp, synthetic construct
  source 1..19: mol_type = other DNA
gcggtttttagttcgcggg

SEQ ID NO: 272 - DNA, 27 bp, synthetic construct
  source 1..27: mol_type = other DNA
aacaaataattaacaaacaacgcctcc

SEQ ID NO: 273 - DNA, 24 bp, synthetic construct
  source 1..24: mol_type = other DNA
tcgttcggcgtatttatttcgtat

SEQ ID NO: 274 - DNA, 19 bp, synthetic construct
  source 1..19: mol_type = other DNA
cgcgaaaaacttcctccga

SEQ ID NO: 275 - DNA, 20 bp, synthetic construct
  source 1..20: mol_type = other DNA
ggattggtgagattcgggac

SEQ ID NO: 276 - DNA, 21 bp, synthetic construct
  source 1..21: mol_type = other DNA
cccgaaaccaaaaaacaacga

SEQ ID NO: 277 - DNA, 21 bp, synthetic construct
  source 1..21: mol_type = other DNA
cggggagtttcgtttgtatcg

SEQ ID NO: 278 - DNA, 26 bp, synthetic construct
  source 1..26: mol_type = other DNA
cgaataacgactacgaactttaaacg

SEQ ID NO: 279 - DNA, 28 bp, synthetic construct
  source 1..28: mol_type = other DNA
cgtagttatagatttattagagagggcg

SEQ ID NO: 280 - DNA, 26 bp, synthetic construct
  source 1..26: mol_type = other DNA
ctaaaacgataaaaaaacgcgaaacg

SEQ ID NO: 281 - DNA, 23 bp, synthetic construct
  source 1..23: mol_type = other DNA
tggtagttttcggtatcgacgag

SEQ ID NO: 282 - DNA, 20 bp, synthetic construct
  source 1..20: mol_type = other DNA
taactctacgcgcaaaacgc

SEQ ID NO: 283 - DNA, 20 bp, synthetic construct
  source 1..20: mol_type = other DNA
gggatttttagcggaagcga

SEQ ID NO: 284 - DNA, 23 bp, synthetic construct
  source 1..23: mol_type = other DNA
cgacgaactatccgactatcact

SEQ ID NO: 285 - DNA, 21 bp, synthetic construct
  source 1..21: mol_type = other DNA
cggaggaatcgggtagaatcg

SEQ ID NO: 286 - DNA, 22 bp, synthetic construct
  source 1..22: mol_type = other DNA
actctcccataacaacgactcc

SEQ ID NO: 287 - DNA, 17 bp, synthetic construct
  source 1..17: mol_type = other DNA
cgcgtagggcgagtttc

SEQ ID NO: 288 - DNA, 18 bp, synthetic construct
  source 1..18: mol_type = other DNA
gcctcctcctacctctcg

SEQ ID NO: 289 - DNA, 22 bp, synthetic construct
  source 1..22: mol_type = other DNA
tcggcggtttttagtaaaagcg

SEQ ID NO: 290 - DNA, 19 bp, synthetic construct
  source 1..19: mol_type = other DNA
aaatctcccgtcccactcc

SEQ ID NO: 291 - DNA, 20 bp, synthetic construct
  source 1..20: mol_type = other DNA
ggtatattcgcgtaggtgcg

SEQ ID NO: 292 - DNA, 25 bp, synthetic construct
  source 1..25: mol_type = other DNA
aaccgaataccgaaatctataaccc

SEQ ID NO: 293 - DNA, 21 bp, synthetic construct
  source 1..21: mol_type = other DNA
gtagttgggttgtagtgcgtg

SEQ ID NO: 294 - DNA, 19 bp, synthetic construct
  source 1..19: mol_type = other DNA
cccgaacttcccatcgaac

SEQ ID NO: 295 - DNA, 23 bp, synthetic construct
  source 1..23: mol_type = other DNA
ttcgtacggttttttcgttttcg

SEQ ID NO: 296 - DNA, 26 bp, synthetic construct
  source 1..26: mol_type = other DNA
ccaccacgtaataattcttctcgaaa

SEQ ID NO: 297 - DNA, 24 bp, synthetic construct
  source 1..24: mol_type = other DNA
cggtttgatattattcggggttcg

SEQ ID NO: 298 - DNA, 23 bp, synthetic construct
  source 1..23: mol_type = other DNA
aattaacgccccctataacatcc

SEQ ID NO: 299 - DNA, 25 bp, synthetic construct
  source 1..25: mol_type = other DNA
cgcgatagcgttttttattgtcgcg

SEQ ID NO: 300 - DNA, 21 bp, synthetic construct
  source 1..21: mol_type = other DNA
cgcacgaccgtaaaatactcg

SEQ ID NO: 301 - DNA, 24 bp, synthetic construct
  source 1..24: mol_type = other DNA
cggttcgtagattgttttagagcg

SEQ ID NO: 302 - DNA, 32 bp, synthetic construct
  source 1..32: mol_type = other DNA
caaataccgtcgaaaaaaaactaaatcaaaac

SEQ ID NO: 303 - DNA, 21 bp, synthetic construct
  source 1..21: mol_type = other DNA
cgttgtaggacggttatgtcg

SEQ ID NO: 304 - DNA, 25 bp, synthetic construct
  source 1..25: mol_type = other DNA
catcctactcttcgaaataaaccgc

SEQ ID NO: 305 - DNA, 26 bp, synthetic construct
  source 1..26: mol_type = other DNA
agtcgtttttttaggtagtttaggcg

SEQ ID NO: 306 - DNA, 19 bp, synthetic construct
  source 1..19: mol_type = other DNA
cgacctttacaatcgccgc

SEQ ID NO: 307 - DNA, 19 bp, synthetic construct
  source 1..19: mol_type = other DNA
agattggcgcgtaaaagcg

SEQ ID NO: 308 - DNA, 17 bp, synthetic construct
  source 1..17: mol_type = other DNA
accgaccccgaaaaacg

SEQ ID NO: 309 - DNA, 18 bp, synthetic construct
  source 1..18: mol_type = other DNA
cgttttcggagtcgcgtg

SEQ ID NO: 310 - DNA, 26 bp, synthetic construct
  source 1..26: mol_type = other DNA
aaccatacttatccgaacgtctaaac

SEQ ID NO: 311 - DNA, 21 bp, synthetic construct
  source 1..21: mol_type = other DNA
gagtagggttattttcgcggg

SEQ ID NO: 312 - DNA, 29 bp, synthetic construct
  source 1..29: mol_type = other DNA
ctacttttttcccgacaaaataaaaacgt

SEQ ID NO: 313 - DNA, 21 bp, synthetic construct
  source 1..21: mol_type = other DNA
ttttcgcgtatttcgtggttc

SEQ ID NO: 314 - DNA, 16 bp, synthetic construct
  source 1..16: mol_type = other DNA
aacgccgccgtattcg

SEQ ID NO: 315 - DNA, 17 bp, synthetic construct
  source 1..17: mol_type = other DNA
tcgcgttggaagttgcg

SEQ ID NO: 316 - DNA, 25 bp, synthetic construct
  source 1..25: mol_type = other DNA
cgtattctaaacgctaaaaaaccgc

SEQ ID NO: 317 - DNA, 17 bp, synthetic construct
  source 1..17: mol_type = other DNA
cggcggtagttttgcgg

SEQ ID NO: 318 - DNA, 25 bp, synthetic construct
  source 1..25: mol_type = other DNA
ctactaaacaaaaaccaacacgtcc

SEQ ID NO: 319 - DNA, 24 bp, synthetic construct
  source 1..24: mol_type = other DNA
ggtaggtttgttagttggttttcg

SEQ ID NO: 320 - DNA, 20 bp, synthetic construct
  source 1..20: mol_type = other DNA
cgctatctctacgaccgcct

SEQ ID NO: 321 - DNA, 17 bp, synthetic construct
  source 1..17: mol_type = other DNA
cggaggtagcgggcgag

SEQ ID NO: 322 - DNA, 17 bp, synthetic construct
  source 1..17: mol_type = other DNA
cactcaccgcttccgcc

SEQ ID NO: 323 - DNA, 16 bp, synthetic construct
  source 1..16: mol_type = other DNA
ggcgttcgtttcggcg

SEQ ID NO: 324 - DNA, 16 bp, synthetic construct
  source 1..16: mol_type = other DNA
cgcttctccgacgccc

SEQ ID NO: 325 - DNA, 20 bp, synthetic construct
  source 1..20: mol_type = other DNA
gcggttgagtttttggtcgg

SEQ ID NO: 326 - DNA, 24 bp, synthetic construct
  source 1..24: mol_type = other DNA
ccccgtataactaaaaacgacgac

SEQ ID NO: 327 - DNA, 31 bp, synthetic construct
  source 1..31: mol_type = other DNA
cgttttttttatgtagtaagcgatttttcgc

SEQ ID NO: 328 - DNA, 17 bp, synthetic construct
  source 1..17: mol_type = other DNA
aaacgacgacgaacgcc

SEQ ID NO: 329 - DNA, 24 bp, synthetic construct
  source 1..24: mol_type = other DNA
gcgttttggttttttcgtttcgag

SEQ ID NO: 330 - DNA, 21 bp, synthetic construct
  source 1..21: mol_type = other DNA
gaacgacgaaactaaaaccgc

SEQ ID NO: 331 - DNA, 27 bp, synthetic construct
  source 1..27: mol_type = other DNA
cgttttttggtagtttttttcgagtcg

SEQ ID NO: 332 - DNA, 22 bp, synthetic construct
  source 1..22: mol_type = other DNA
gcttaaacgtaaccgaaacgcc

SEQ ID NO: 333 - DNA, 19 bp, synthetic construct
  source 1..19: mol_type = other DNA
ggttgcgcgtcgttttttc

SEQ ID NO: 334 - DNA, 19 bp, synthetic construct
  source 1..19: mol_type = other DNA
cccgacgcgtttaaatcgt

SEQ ID NO: 335 - DNA, 28 bp, synthetic construct
  source 1..28: mol_type = other DNA
ggatgggaatagttaagttttagtcgtt

SEQ ID NO: 336 - DNA, 27 bp, synthetic construct
  source 1..27: mol_type = other DNA
tccaacattacatacaacactaacgtc

SEQ ID NO: 337 - DNA, 21 bp, synthetic construct
  source 1..21: mol_type = other DNA
ggttaggggtggagttcgtta

SEQ ID NO: 338 - DNA, 24 bp, synthetic construct
  source 1..24: mol_type = other DNA
actccgaactctactcatcctttc

SEQ ID NO: 339 - DNA, 21 bp, synthetic construct
  source 1..21: mol_type = other DNA
taggttggagattttgacgcg

SEQ ID NO: 340 - DNA, 22 bp, synthetic construct
  source 1..22: mol_type = other DNA
cgaaacctaaaaacgccgaaac

SEQ ID NO: 341 - DNA, 25 bp, synthetic construct
  source 1..25: mol_type = other DNA
ggttggtagtcgtttttacgttttc

SEQ ID NO: 342 - DNA, 30 bp, synthetic construct
  source 1..30: mol_type = other DNA
caaaacccatctaattacaaaatacctcga

SEQ ID NO: 343 - DNA, 19 bp, synthetic construct
  source 1..19: mol_type = other DNA
tggagttatcggaaggcga

SEQ ID NO: 344 - DNA, 18 bp, synthetic construct
  source 1..18: mol_type = other DNA
cgaactcccgaaacgacg

SEQ ID NO: 345 - DNA, 27 bp, synthetic construct
  source 1..27: mol_type = other DNA
ggaaatggtttagagttttggatttcg

SEQ ID NO: 346 - DNA, 27 bp, synthetic construct
  source 1..27: mol_type = other DNA
ttctaaaaaatactttcgataccgaca

SEQ ID NO: 347 - DNA, 29 bp, synthetic construct
  source 1..29: mol_type = other DNA
gtggtttgaagagttgttagttcgtttag

SEQ ID NO: 348 - DNA, 23 bp, synthetic construct
  source 1..23: mol_type = other DNA
gccaaaaattcgattccaacgca

SEQ ID NO: 349 - DNA, 22 bp, synthetic construct
  source 1..22: mol_type = other DNA
aaggtgggttgtttgaagaagc

SEQ ID NO: 350 - DNA, 22 bp, synthetic construct
  source 1..22: mol_type = other DNA
accctccgacaaaaaaacgtac

SEQ ID NO: 351 - DNA, 21 bp, synthetic construct
  source 1..21: mol_type = other DNA
gcgagaaggttttgtcgtaga

SEQ ID NO: 352 - DNA, 19 bp, synthetic construct
  source 1..19: mol_type = other DNA
acctactcacgcacaacct

SEQ ID NO: 353 - DNA, 17 bp, synthetic construct
  source 1..17: mol_type = other DNA
cgcggtgaggaagatcg

SEQ ID NO: 354 - DNA, 18 bp, synthetic construct
  source 1..18: mol_type = other DNA
acgccacctacgactacg

SEQ ID NO: 355 - DNA, 21 bp, synthetic construct
  source 1..21: mol_type = other DNA
gtacggttattggttgagcgg

SEQ ID NO: 356 - DNA, 19 bp, synthetic construct
  source 1..19: mol_type = other DNA
ccgaatcctcctccaaacg

SEQ ID NO: 357 - DNA, 28 bp, synthetic construct
  source 1..28: mol_type = other DNA
ggaatttaattggagagaaattttggcg

SEQ ID NO: 358 - DNA, 27 bp, synthetic construct
  source 1..27: mol_type = other DNA
ccaaaatttccctcatctatatacgcc

SEQ ID NO: 359 - DNA, 17 bp, synthetic construct
  source 1..17: mol_type = other DNA
gttgcgggtcgggttgc

SEQ ID NO: 360 - DNA, 26 bp, synthetic construct
  source 1..26: mol_type = other DNA
gcaaaaacaaaaaacgcgtaaaaacc

SEQ ID NO: 361 - DNA, 21 bp, synthetic construct
  source 1..21: mol_type = other DNA
gtcgtcgttgtcgtagttgtc

SEQ ID NO: 362 - DNA, 20 bp, synthetic construct
  source 1..20: mol_type = other DNA
cgtaaaaaccgaacgacgcg

SEQ ID NO: 363 - DNA, 26 bp, synthetic construct
  source 1..26: mol_type = other DNA
ttttcgttgattttattcgagtcgtc

SEQ ID NO: 364 - DNA, 22 bp, synthetic construct
  source 1..22: mol_type = other DNA
gaaccctcttcaaataaaccgc

SEQ ID NO: 365 - DNA, 21 bp, synthetic construct
  source 1..21: mol_type = other DNA
gattaaatagtcggggtcgcg

SEQ ID NO: 366 - DNA, 19 bp, synthetic construct
  source 1..19: mol_type = other DNA
attctccaacgccaaccac

SEQ ID NO: 367 - DNA, 24 bp, synthetic construct
  source 1..24: mol_type = other DNA
cgcgccgaggcgcgcttccactcc

SEQ ID NO: 368 - DNA, 33 bp, synthetic construct
  source 1..33: mol_type = other DNA
aggccacggacggcggcgtttttttttatgttg

SEQ ID NO: 369 - DNA, 39 bp, synthetic construct
  source 1..39: mol_type = other DNA
aggccacggacgcaaccgaactaaaaaaaaaaactaacg

SEQ ID NO: 370 - DNA, 27 bp, synthetic construct
  source 1..27: mol_type = other DNA
cgcgccgagggcgcgtaagattttcgg

SEQ ID NO: 371 - DNA, 25 bp, synthetic construct
  source 1..25: mol_type = other DNA
cgcgccgagggcgggtagtagtgcg

SEQ ID NO: 372 - DNA, 31 bp, synthetic construct
  source 1..31: mol_type = other DNA
cgcgccgaggcgaccgaaaaatcgaaaaaca

SEQ ID NO: 373 - DNA, 30 bp, synthetic construct
  source 1..30: mol_type = other DNA
cgcgccgagggctaacgcgaataaaacacg

SEQ ID NO: 374 - DNA, 29 bp, synthetic construct
  source 1..29: mol_type = other DNA
cgcgccgaggttttcgttttcggtttcgg

SEQ ID NO: 375 - DNA, 28 bp, synthetic construct
  source 1..28: mol_type = other DNA
cgcgccgaggccgtacctattaactccg

SEQ ID NO: 376 - DNA, 31 bp, synthetic construct
  source 1..31: mol_type = other DNA
aggccacggacgtcgttttttttttgcgggt

SEQ ID NO: 377 - DNA, 30 bp, synthetic construct
  source 1..30: mol_type = other DNA
aggccacggacgcgtattggcgcgatttag

SEQ ID NO: 378 - DNA, 33 bp, synthetic construct
  source 1..33: mol_type = other DNA
aggccacggacggttcgaaaaaaactcgacgaa

SEQ ID NO: 379 - DNA, 30 bp, synthetic construct
  source 1..30: mol_type = other DNA
aggccacggacggccgttctctaacttccg

SEQ ID NO: 380 - DNA, 29 bp, synthetic construct
  source 1..29: mol_type = other DNA
aggccacggacgccgaaatacgaacgcga

SEQ ID NO: 381 - DNA, 29 bp, synthetic construct
  source 1..29: mol_type = other DNA
aggccacggacgtcgttcctcgatttcgc

SEQ ID NO: 382 - DNA, 37 bp, synthetic construct
  source 1..37: mol_type = other DNA
aggccacggacgcgaataaaacctacgaaaaaaaacg

SEQ ID NO: 383 - DNA, 31 bp, synthetic construct
  source 1..31: mol_type = other DNA
aggccacggacggaaaaccgcaaaatcctcg

SEQ ID NO: 384 - DNA, 38 bp, synthetic construct
  source 1..38: mol_type = other DNA
aggccacggacgcgcgaaataaacctataattaactca

SEQ ID NO: 385 - DNA, 29 bp, synthetic construct
  source 1..29: mol_type = other DNA
cgcgccgaggccgaaccaacactcaaaac

SEQ ID NO: 386 - DNA, 26 bp, synthetic construct
  source 1..26: mol_type = other DNA
cgcgccgagggcgcgtttggtttggt

SEQ ID NO: 387 - DNA, 26 bp, synthetic construct
  source 1..26: mol_type = other DNA
aggccacggacgaacgcgctccaacc

SEQ ID NO: 388 - DNA, 31 bp, synthetic construct
  source 1..31: mol_type = other DNA
cgcgccgaggcgcgacgactaaaaaaattca

SEQ ID NO: 389 - DNA, 30 bp, synthetic construct
  source 1..30: mol_type = other DNA
aggccacggacggtcgaaatcgaaacgctc

SEQ ID NO: 390 - DNA, 26 bp, synthetic construct
  source 1..26: mol_type = other DNA
cgcgccgaggccgcgactacctcttc

SEQ ID NO: 391 - DNA, 30 bp, synthetic construct
  source 1..30: mol_type = other DNA
aggccacggacgcgacgtcctacaaaacca

SEQ ID NO: 392 - DNA, 25 bp, synthetic construct
  source 1..25: mol_type = other DNA
cgcgccgaggggcggtagttgttgc

SEQ ID NO: 393 - DNA, 24 bp, synthetic construct
  source 1..24: mol_type = other DNA
cgcgccgagggcgaaaaccccgcc

SEQ ID NO: 394 - DNA, 32 bp, synthetic construct
  source 1..32: mol_type = other DNA
cgcgccgaggcgtctaactaaacgcgataaac

SEQ ID NO: 395 - DNA, 29 bp, synthetic construct
  source 1..29: mol_type = other DNA
cgcgccgagggcggttttagcgatgaatc

SEQ ID NO: 396 - DNA, 24 bp, synthetic construct
  source 1..24: mol_type = other DNA
cgcgccgaggcgattcgagggcgt

SEQ ID NO: 397 - DNA, 27 bp, synthetic construct
  source 1..27: mol_type = other DNA
aggccacggacgcgcgacgcaaaactc

SEQ ID NO: 398 - DNA, 26 bp, synthetic construct
  source 1..26: mol_type = other DNA
aggccacggacggcggcggttggatt

SEQ ID NO: 399 - DNA, 31 bp, synthetic construct
  source 1..31: mol_type = other DNA
aggccacggacgtcccgaaacgaacgataaa

SEQ ID NO: 400 - DNA, 24 bp, synthetic construct
  source 1..24: mol_type = other DNA
cgcgccgaggcgccgtcgcactac

SEQ ID NO: 401 - DNA, 26 bp, synthetic construct
  source 1..26: mol_type = other DNA
aggccacggacgcgcgactccccact

SEQ ID NO: 402 - DNA, 32 bp, synthetic construct
  source 1..32: mol_type = other DNA
aggccacggacgcgcaaataataacgcgaacg

SEQ ID NO: 403 - DNA, 33 bp, synthetic construct
  source 1..33: mol_type = other DNA
aggccacggacgcgttcgaggttagttttttgg

SEQ ID NO: 404 - DNA, 27 bp, synthetic construct
  source 1..27: mol_type = other DNA
aggccacggacgcgtacgtaacccgcg

SEQ ID NO: 405 - DNA, 29 bp, synthetic construct
  source 1..29: mol_type = other DNA
cgcgccgaggcgctactaaccataaccgc

SEQ ID NO: 406 - DNA, 30 bp, synthetic construct
  source 1..30: mol_type = other DNA
cgcgccgaggcgttttcgtttgattcggtt

SEQ ID NO: 407 - DNA, 29 bp, synthetic construct
  source 1..29: mol_type = other DNA
cgcgccgaggtcggtgattgatgtgtgcg

SEQ ID NO: 408 - DNA, 31 bp, synthetic construct
  source 1..31: mol_type = other DNA
cgcgccgaggcgtaactccatctcgataacc

SEQ ID NO: 409 - DNA, 26 bp, synthetic construct
  source 1..26: mol_type = other DNA
cgcgccgaggcgaccgcgaaaaaacg

SEQ ID NO: 410 - DNA, 28 bp, synthetic construct
  source 1..28: mol_type = other DNA
aggccacggacgcgcgttggaagtttcg

SEQ ID NO: 411 - DNA, 26 bp, synthetic construct
  source 1..26: mol_type = other DNA
cgcgccgagggcgcgaacacaaaacg

SEQ ID NO: 412 - DNA, 27 bp, synthetic construct
  source 1..27: mol_type = other DNA
cgcgccgaggaccgaaaacgccctaaa

SEQ ID NO: 413 - DNA, 31 bp, synthetic construct
  source 1..31: mol_type = other DNA
cgcgccgagggcgtggagaaatgttagtttg

SEQ ID NO: 414 - DNA, 28 bp, synthetic construct
  source 1..28: mol_type = other DNA
aggccacggacgcgggcggtgaatttgt

SEQ ID NO: 415 - DNA, 29 bp, synthetic construct
  source 1..29: mol_type = other DNA
aggccacggacgacgtcgtatttgtggcg

SEQ ID NO: 416 - DNA, 27 bp, synthetic construct
  source 1..27: mol_type = other DNA
aggccacggacggcgttgtttagcgcg

SEQ ID NO: 417 - DNA, 30 bp, synthetic construct
  source 1..30: mol_type = other DNA
aggccacggacggatcccgcaaatcaacac

SEQ ID NO: 418 - DNA, 25 bp, synthetic construct
  source 1..25: mol_type = other DNA
cgcgccgaggcgatccccaactccc

SEQ ID NO: 419 - DNA, 30 bp, synthetic construct
  source 1..30: mol_type = other DNA
cgcgccgaggcgcttctaaaacctcgatcc

SEQ ID NO: 420 - DNA, 31 bp, synthetic construct
  source 1..31: mol_type = other DNA
cgcgccgaggcgcggtcgttaatatgtattc

SEQ ID NO: 421 - DNA, 32 bp, synthetic construct
  source 1..32: mol_type = other DNA
aggccacggacgcgtttggcgtagatataagc

SEQ ID NO: 422 - DNA, 32 bp, synthetic construct
  source 1..32: mol_type = other DNA
aggccacggacgcgaactacgaaaacaacctc

SEQ ID NO: 423 - DNA, 20 bp, synthetic construct
  source 1..20: mol_type = other DNA
ggattggtgagattcgggac

SEQ ID NO: 424 - DNA, 21 bp, synthetic construct
  source 1..21: mol_type = other DNA
cccgaaaccaaaaaacaacga

SEQ ID NO: 425 - DNA, 21 bp, synthetic construct
  source 1..21: mol_type = other DNA
gagtagggttattttcgcggg

SEQ ID NO: 426 - DNA, 29 bp, synthetic construct
  source 1..29: mol_type = other DNA
ctacttttttcccgacaaaataaaaacgt

SEQ ID NO: 427 - DNA, 25 bp, synthetic construct
  source 1..25: mol_type = other DNA
cgcgatagcgttttttattgtcgcg

SEQ ID NO: 428 - DNA, 21 bp, synthetic construct
  source 1..21: mol_type = other DNA
cgcacgaccgtaaaatactcg

SEQ ID NO: 429 - DNA, 20 bp, synthetic construct
  source 1..20: mol_type = other DNA
cggttcgttgcgtttatcga

SEQ ID NO: 430 - DNA, 20 bp, synthetic construct
  source 1..20: mol_type = other DNA
cccccgaactactctacgcg

SEQ ID NO: 431 - DNA, 21 bp, synthetic construct
  source 1..21: mol_type = other DNA
gattaaatagtcggggtcgcg

SEQ ID NO: 432 - DNA, 19 bp, synthetic construct
  source 1..19: mol_type = other DNA
attctccaacgccaaccac

SEQ ID NO: 433 - DNA, 21 bp, synthetic construct
  source 1..21: mol_type = other DNA
gtacggttattggttgagcgg

SEQ ID NO: 434 - DNA, 19 bp, synthetic construct
  source 1..19: mol_type = other DNA
ccgaatcctcctccaaacg

SEQ ID NO: 435-436 - skipped (2 entries)

SEQ ID NO: 437 - DNA, 20 bp, synthetic construct
  source 1..20: mol_type = other DNA
gggatttttagcggaagcga

SEQ ID NO: 438 - DNA, 23 bp, synthetic construct
  source 1..23: mol_type = other DNA
cgacgaactatccgactatcact

SEQ ID NO: 439 - DNA, 21 bp, synthetic construct
  source 1..21: mol_type = other DNA
ggttaggggtggagttcgtta

SEQ ID NO: 440 - DNA, 24 bp, synthetic construct
  source 1..24: mol_type = other DNA
actccgaactctactcatcctttc

SEQ ID NO: 441 - DNA, 22 bp, synthetic construct
  source 1..22: mol_type = other DNA
tcggcggtttttagtaaaagcg

SEQ ID NO: 442 - DNA, 19 bp, synthetic construct
  source 1..19: mol_type = other DNA
aaatctcccgtcccactcc

SEQ ID NO: 443 - DNA, 25 bp, synthetic construct
  source 1..25: mol_type = other DNA
ggttggtagtcgtttttacgttttc

SEQ ID NO: 444 - DNA, 30 bp, synthetic construct; identical to SEQ ID NO: 342
  source 1..30: mol_type = other DNA

SEQ ID NO: 445 - DNA, 19 bp, synthetic construct
  source 1..19: mol_type = other DNA
tggagttatcggaaggcga

SEQ ID NO: 446 - DNA, 18 bp, synthetic construct
  source 1..18: mol_type = other DNA
cgaactcccgaaacgacg

SEQ ID NO: 447 - DNA, 26 bp, synthetic construct
  source 1..26: mol_type = other DNA
ttttcgttgattttattcgagtcgtc

SEQ ID NO: 448 - DNA, 22 bp, synthetic construct
  source 1..22: mol_type = other DNA
gaaccctcttcaaataaaccgc

SEQ ID NO: 449 - DNA, 28 bp, synthetic construct
  source 1..28: mol_type = other DNA
gattatattcggattttgtttatcgcgt

SEQ ID NO: 450 - DNA, 18 bp, synthetic construct
  source 1..18: mol_type = other DNA
gactcttcctacccgcga

SEQ ID NO: 451 - DNA, 30 bp, synthetic construct; identical to SEQ ID NO: 377
  source 1..30: mol_type = other DNA

SEQ ID NO: 452 - DNA, 29 bp, synthetic construct
  source 1..29: mol_type = other DNA
cgcgccgagggcggttttagcgatgaatc

SEQ ID NO: 453 - DNA, 30 bp, synthetic construct; identical to SEQ ID NO: 389
  source 1..30: mol_type = other DNA

SEQ ID NO: 454 - DNA, 30 bp, synthetic construct; identical to SEQ ID NO: 373
  source 1..30: mol_type = other DNA

SEQ ID NO: 455 - DNA, 32 bp, synthetic construct; identical to SEQ ID NO: 422
  source 1..32: mol_type = other DNA

SEQ ID NO: 456 - DNA, 30 bp, synthetic construct; identical to SEQ ID NO: 417
  source 1..30: mol_type = other DNA

SEQ ID NO: 457 - skipped

SEQ ID NO: 458 - DNA, 27 bp, synthetic construct
  source 1..27: mol_type = other DNA
cgcgccgaggtcgttcctcgatttcgc

SEQ ID NO: 459 - DNA, 31 bp, synthetic construct; identical to SEQ ID NO: 408
  source 1..31: mol_type = other DNA

SEQ ID NO: 460 - DNA, 36 bp, synthetic construct
  source 1..36: mol_type = other DNA
cgcgccgaggcgcgaaataaacctataattaactca

SEQ ID NO: 461 - DNA, 28 bp, synthetic construct
  source 1..28: mol_type = other DNA
aggccacggacgcgcgttggaagtttcg

SEQ ID NO: 462 - DNA, 26 bp, synthetic construct
  source 1..26: mol_type = other DNA
cgcgccgagggcgcgaacacaaaacg

SEQ ID NO: 463 - DNA, 32 bp, synthetic construct; identical to SEQ ID NO: 421
  source 1..32: mol_type = other DNA

SEQ ID NO: 464 - DNA, 29 bp, synthetic construct
  source 1..29: mol_type = other DNA
cgcgccgaggttttcgttttcggtttcgg

(19) United States
(12) Patent Application Publication
(10) Pub. No.: US 2025/0327134 A1
(43) Pub. Date: Oct. 23, 2025

(54) DETECTING BREAST CANCER

(71) Applicants: Mayo Foundation for Medical Education and Research, Rochester, MN (US); Exact Sciences Corporation, Madison, WI (US)

(72) Inventors: David A. Ahlquist, Rochester, MN (US); William R. Taylor, Lake City, MN (US); Douglas W. Mahoney, Elgin, MN (US); Tracy C. Yab, Rochester, MN (US); John B. Kisiel, Rochester, MN (US); Hatim T. Allawi, Middleton, WI (US); Graham P. Lidgard, Middleton, WI (US); Michael W. Kaiser, Stoughton, WI (US)

(21) Appl. No.: 19/199,792

(22) Filed: May 6, 2025

Related U.S. Application Data
(63) Continuation of application No. 17/205,796, filed on Mar. 18, 2021, now Pat. No. 12,325,878.
Application No. 17/205,796 is a continuation of application No. 16/202,935, filed on Nov. 28, 2018, now Pat. No. 10,975,443.
(60) Provisional application No. 62/592,828, filed on Nov. 30, 2017.

(51) Int. Cl. C12Q 1/6886; C12Q 1/6806; C12Q 1/6809; C12Q 1/6811; C12Q 1/682; C12Q 1/6827; C12Q 1/6837; C12Q 1/6853; G16H 50/30

(52) U.S. Cl. CPC C12Q 1/6886; C12Q 1/6806; C12Q 1/6809; C12Q 1/6811; C12Q 1/682; C12Q 1/6827; C12Q 1/6837; C12Q 1/6853; G16H 50/30; C12Q 2537/143; C12Q 2600/106; C12Q 2600/112; C12Q 2600/118; C12Q 2600/154; C12Q 2600/156; C12Q 2600/158; C12Q 2600/16

(57) ABSTRACT

Provided herein is technology for breast cancer screening and particularly, but not exclusively, to methods, compositions, and related uses for detecting the presence of breast cancer.

### CROSS REFERENCE TO RELATED APPLICATION

This application is a continuation of U.S. patent application Ser. No. 17/205,796, filed Mar. 18, 2021, which is a continuation of U.S. patent application Ser. No. 16/202,935, filed Nov. 28, 2018, now U.S. Pat. No. 10,975,443, which claims priority to and the benefit of U.S. Provisional Application No. 62/592,828, filed Nov. 30, 2017, the contents of which are hereby incorporated by reference in their entireties.

### SEQUENCE LISTING

The text of the computer readable sequence listing filed herewith, titled “35440-308_SEQUENCE_LISTING”, created May 6, 2025, having a file size of 409,776 bytes, is hereby incorporated by reference in its entirety.

### FIELD OF INVENTION

Provided herein is technology for breast cancer screening and particularly, but not exclusively, to methods, compositions, and related uses for detecting the presence of breast cancer.

### BACKGROUND

Breast cancer affects approximately 230,000 US women per year and claims about 40,000 lives every year. Although carriers of germline mutations in BRCA1 and BRCA2 genes are known to be at high risk of breast cancer, most women who get breast cancer do not have a mutation in one of these genes and there is limited ability to accurately identify women at increased risk of breast cancer. Effective prevention therapies exist, but current risk prediction models do not accurately identify the majority of women at increased risk of breast cancer (see, e.g., Pankratz V S, et al., J Clin Oncol 2008 Nov. 20; 26 (33): 5374-9).

Improved methods for detecting breast cancer are needed.

The present invention addresses these needs.

### SUMMARY

Methylated DNA has been studied as a potential class of biomarkers in the tissues of most tumor types. In many instances, DNA methyltransferases add a methyl group to DNA at cytosine-phosphate-guanine (CpG) island sites as an epigenetic control of gene expression. In a biologically attractive mechanism, acquired methylation events in promoter regions of tumor suppressor genes are thought to silence expression, thus contributing to oncogenesis. DNA methylation may be a more chemically and biologically stable diagnostic tool than RNA or protein expression (Laird (2010) Nat Rev Genet 11:191-203). Furthermore, in other cancers like sporadic colon cancer, methylation markers offer excellent specificity and are more broadly informative and sensitive than are individual DNA mutations (Zou et al (2007) Cancer Epidemiol Biomarkers Prev 16:2686-96).

Analysis of CpG islands has yielded important findings when applied to animal models and human cell lines. For example, Zhang and colleagues found that amplicons from different parts of the same CpG island may have different levels of methylation (Zhang et al. (2009) PLOS Genet 5: e1000438). Further, methylation levels were distributed bi-modally between highly methylated and unmethylated sequences, further supporting the binary switch-like pattern of DNA methyltransferase activity (Zhang et al. (2009) PLOS Genet 5: e1000438). Analysis of murine tissues in vivo and cell lines in vitro demonstrated that only about 0.3% of high CpG density promoters (HCP, defined as having >7% CpG sequence within a 300 base pair region) were methylated, whereas areas of low CpG density (LCP, defined as having <5% CpG sequence within a 300 base pair region) tended to be frequently methylated in a dynamic tissue-specific pattern (Meissner et al. (2008) Nature 454:766-70). HCPs include promoters for ubiquitous housekeeping genes and highly regulated developmental genes. Among the HCP sites methylated at >50% were several established markers such as Wnt 2, NDRG2, SFRP2, and BMP3 (Meissner et al. (2008) Nature 454:766-70).

Epigenetic methylation of DNA at cytosine-phosphate-guanine (CpG) island sites by DNA methyltransferases has been studied as a potential class of biomarkers in the tissues of most tumor types. In a biologically attractive mechanism, acquired methylation events in promotor regions of tumor suppressor genes are thought to silence expression, contributing to oncogenesis. DNA methylation may be a more chemically and biologically stable diagnostic tool than RNA or protein expression. Furthermore, in other cancers like sporadic colon cancer, aberrant methylation markers are more broadly informative and sensitive than are individual DNA mutations and offer excellent specificity.

Several methods are available to search for novel methylation markers. While micro-array based interrogation of CpG methylation is a reasonable, high-throughput approach, this strategy is biased towards known regions of interest, mainly established tumor suppressor promotors. Alternative methods for genome-wide analysis of DNA methylation have been developed in the last decade. There are three basic approaches. The first employs digestion of DNA by restriction enzymes which recognize specific methylated sites, followed by several possible analytic techniques which provide methylation data limited to the enzyme recognition site or the primers used to amplify the DNA in quantification steps (such as methylation-specific PCR; MSP). A second approach enriches methylated fractions of genomic DNA using anti-bodies directed to methyl-cytosine or other methylation-specific binding domains followed by microarray analysis or sequencing to map the fragment to a reference genome. This approach does not provide single nucleotide resolution of all methylated sites within the fragment. A third approach begins with bisulfite treatment of the DNA to convert all unmethylated cytosines to uracil, followed by restriction enzyme digestion and complete sequencing of all fragments after coupling to an adapter ligand. The choice of restriction enzymes can enrich the fragments for CpG dense regions, reducing the number of redundant sequences which may map to multiple gene positions during analysis.

RRBS yields CpG methylation status data at single nucleotide resolution of 80-90% of all CpG islands and a majority of tumor suppressor promoters at medium to high read coverage. In cancer case-control studies, analysis of these reads results in the identification of differentially methylated regions (DMRs). In previous RRBS analysis of pancreatic cancer specimens, hundreds of DMRs were uncovered, many of which had never been associated with carcinogenesis and many of which were unannotated. Further validation studies on independent tissue samples sets confirmed marker CpGs which were 100% sensitive and specific in terms of performance.

Provided herein is technology for breast cancer screening and particularly, but not exclusively, to methods, compositions, and related uses for detecting the presence of breast cancer.

Indeed, as described in Examples I, II and III, experiments conducted during the course for identifying embodiments for the present invention identified a novel set of differentially methylated regions (DMRs) for discriminating cancer of the breast derived DNA from non-neoplastic control DNA.

Such experiments list and describe 375 novel DNA methylation markers distinguishing breast cancer tissue from benign breast tissue (see, Tables 2 and 5, Examples I, II and III).

From these 375 novel DNA methylation markers, further experiments identified the following markers and/or panels of markers capable of distinguishing breast cancer tissue from benign breast tissue:
ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23 D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1 (see, Table 4, Example II); and
ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B (see, Table 9, Example III).

From these 375 novel DNA methylation markers, further experiments identified the following markers and/or panels of markers for detecting breast cancer in blood samples (e.g., plasma samples, whole blood samples, serum samples):
CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B (see, Table 14, Example III).

As described herein, the technology provides a number of methylated DNA markers and subsets thereof (e.g., sets of 2, 3, 4, 5, 6, 7, or 8 markers) with high discrimination for breast cancer overall. Experiments applied a selection filter to candidate markers to identify markers that provide a high signal to noise ratio and a low background level to provide high specificity for purposes of breast cancer screening or diagnosis.

In some embodiments, the technology is related to assessing the presence of and methylation state of one or more of the markers identified herein in a biological sample (e.g., breast tissue, plasma sample). These markers comprise one or more differentially methylated regions (DMR) as discussed herein, e.g., as provided in Tables 2 and 5. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14:7421-7427) and another example involves methylation-sensitive arbitrarily primed PCR (Gonzalgo et al. (1997) (Cancer Res. 57:594-599). In some embodiments, changes in methylation patterns at specific CpG sites are monitored by digestion of genomic DNA with methylation-sensitive restriction enzymes followed by Southern analysis of the regions of interest (digestion-Southern method). In some embodiments, analyzing changes in methylation patterns involves a PCR-based process that involves digestion of genomic DNA with methylation-sensitive restriction enzymes or methylation-dependent restriction enzymes prior to PCR amplification (Singer-Sam et al. (1990) Nucl. Acids Res. 18:687). In addition, other techniques have been reported that utilize bisulfite treatment of DNA as a starting point for methylation analysis. These include methylation-specific PCR (MSP) (Herman et al. (1992) Proc. Natl. Acad. Sci. USA 93:9821-9826) and restriction enzyme digestion of PCR products amplified from bisulfite-converted DNA (Sadri and Hornsby (1996) Nucl. Acids Res. 24:5058-5059; and Xiong and Laird (1997) Nucl. Acids Res. 25:2532-2534). PCR techniques have been developed for detection of gene mutations (Kuppuswamy et al. (1991) Proc. Natl. Acad. Sci. USA 88:1143-1147) and quantification of allelic-specific expression (Szabo and Mann (1995) Genes Dev. 9: 3097-3108; and Singer-Sam et al. (1992) POR Methods Appl. 1:160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated and the resulting methylation-specific sequence is determined using either a real-time PCR or other exponential amplification, e.g., a QuARTS assay (e.g., as provided by U.S. Pat. No. 8,361,720; and U.S. Pat. Appl. Pub. Nos. 2012/0122088 and 2012/0122106, incorporated herein by reference).

For example, in some embodiments methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amount of methylated DNAs relative to the total amount of DNAs in the population, e.g., (number of methylated DNAs)/(the number of methylated DNAs+number of unmethylated DNAs)×100.

Also provided herein are compositions and kits for practicing the methods. For example, in some embodiments, reagents (e.g., primers, probes) specific for one or more markers are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, or other assays). In some embodiments, the kits contain a reagent capable of modifying DNA in a methylation-specific manner (e.g., a methylation-sensitive restriction enzyme, a methylation-dependent restriction enzyme, and a bisulfite reagent). In some embodiments, the kits containing one or more reagent necessary, sufficient, or useful for conducting a method are provided. Also provided are reactions mixtures containing the reagents. Further provided are master mix reagent sets containing a plurality of reagents that may be added to each other and/or to a test sample to complete a reaction mixture.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of one or more DMR, e.g., DMR 1-375 as provided in Tables 2 and 5); comparing methylation states (e.g., of one or more DMR, e.g., DMR 1-375 as provided in Tables 2 and 5); generating standard curves; determining a Ct value; calculating a fraction, frequency, or percentage of methylation (e.g., of one or more DMR, e.g., DMR 1-375 as provided in Tables 2 and 5); identifying a CpG island; determining a specificity and/or sensitivity of an assay or marker; calculating an ROC curve and an associated AUC; sequence analysis; all as described herein or is known in the art.

In some embodiments, a microprocessor or computer uses methylation state data in an algorithm to predict a site of a cancer.

In some embodiments, a software or hardware component receives the results of multiple assays and determines a single value result to report to a user that indicates a cancer risk based on the results of the multiple assays (e.g., determining the methylation state of multiple DMR, e.g., as provided in Tables 2 and 5). Related embodiments calculate a risk factor based on a mathematical combination (e.g., a weighted combination, a linear combination) of the results from multiple assays, e.g., determining the methylation states of multiple markers (such as multiple DMR, e.g., as provided in Tables 2 and 5). In some embodiments, the methylation state of a DMR defines a dimension and may have values in a multidimensional space and the coordinate defined by the methylation states of multiple DMR is a result, e.g., to report to a user, e.g., related to a cancer risk.

Some embodiments comprise a storage medium and memory components. Memory components (e.g., volatile and/or nonvolatile memory) find use in storing instructions (e.g., an embodiment of a process as provided herein) and/or data (e.g., a work piece such as methylation measurements, sequences, and statistical descriptions associated therewith). Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, California and Motorola Corporation of Schaumburg, Illinois. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

Accordingly, provided herein is technology related to a method of screening for breast cancer in a sample obtained from a subject, the method comprising assaying a methylation state of a marker in a sample obtained from a subject (e.g., breast tissue) (e.g., plasma sample) and identifying the subject as having breast cancer when the methylation state of the marker is different than a methylation state of the marker assayed in a subject that does not have breast cancer, wherein the marker comprises a base in a differentially methylated region (DMR) selected from a group consisting of DMR 1-375 as provided in Tables 2 and 5.

In some embodiments wherein the sample obtained from the subject is breast tissue and the methylation state of one or more of the following markers is different than a methylation state of the one or more markers assayed in a subject that does not have breast cancer indicates the subject has breast cancer: ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23_D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1 (see, Table 4, Example II).

In some embodiments wherein the sample obtained from the subject is breast tissue and the methylation state of one or more of the following markers is different than a methylation state of the one or more markers assayed in a subject that does not have breast cancer indicates the subject has breast cancer: ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B (see, Table 9, Example III).

In some embodiments wherein the sample obtained from the subject is a blood sample (e.g., plasma, serum, whole blood) and the methylation state of one or more of the following markers is different than a methylation state of the one or more markers assayed in a subject that does not have breast cancer indicates the subject has breast cancer: CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B (see, Table 14, Example III).

The technology is related to identifying and discriminating breast cancer. Some embodiments provide methods comprising assaying a plurality of markers, e.g., comprising assaying 2 to 11 to 100 or 120 or 375 markers.

The technology is not limited in the methylation state assessed. In some embodiments assessing the methylation state of the marker in the sample comprises determining the methylation state of one base. In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

Furthermore, in some embodiments the marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

The technology is not limited by sample type. For example, in some embodiments the sample is a stool sample, a tissue sample (e.g., breast tissue sample), a blood sample (e.g., plasma, serum, whole blood), an excretion, or a urine sample.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc.

The technology provides reagents for detecting a DMR, e.g., in some embodiments are provided a set of oligonucleotides comprising the sequences provided by SEQ ID NO: 1-422 (see, Tables 3, 6, 7, 15 and 16). In some embodiments are provided an oligonucleotide comprising a sequence complementary to a chromosomal region having a base in a DMR, e.g., an oligonucleotide sensitive to methylation state of a DMR.

The technology provides various panels of markers use for identifying breast cancer, e.g., in some embodiments the marker comprises a chromosomal region having an annotation that is ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23 D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1 (see, Table 4, Example II).

The technology provides various panels of markers use for identifying breast cancer, e.g., in some embodiments the marker comprises a chromosomal region having an annotation that is ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B (see, Table 9, Example III).

The technology provides various panels of markers use for identifying breast cancer, e.g., in some embodiments the marker comprises a chromosomal region having an annotation that is CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B (see, Table 14, Example III).

Kit embodiments are provided, e.g., a kit comprising a reagent capable of modifying DNA in a methylation-specific manner (e.g., a methylation-sensitive restriction enzyme, a methylation-dependent restriction enzyme, and a bisulfite reagent); and a control nucleic acid comprising a sequence from a DMR selected from a group consisting of DMR 1-375 (from Tables 2 and 5) and having a methylation state associated with a subject who does not have breast cancer. In some embodiments, kits comprise a reagent capable of modifying DNA in a methylation-specific manner (e.g., a methylation-sensitive restriction enzyme, a methylation-dependent restriction enzyme, and a bisulfite reagent); and a control nucleic acid comprising a sequence from a DMR selected from a group consisting of DMR 1-375 (from Tables 2 and 5) and having a methylation state associated with a subject who has breast cancer. Some kit embodiments comprise a sample collector for obtaining a sample from a subject (e.g., a stool sample; breast tissue sample; plasma sample, serum sample, whole blood sample); a reagent capable of modifying DNA in a methylation-specific manner (e.g., a methylation-sensitive restriction enzyme, a methylation-dependent restriction enzyme, and a bisulfite reagent); and an oligonucleotide as described herein.

The technology is related to embodiments of compositions (e.g., reaction mixtures). In some embodiments are provided a composition comprising a nucleic acid comprising a DMR and a reagent capable of modifying DNA in a methylation-specific manner (e.g., a methylation-sensitive restriction enzyme, a methylation-dependent restriction enzyme, and a bisulfite reagent). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

Additional related method embodiments are provided for screening for breast cancer in a sample obtained from a subject (e.g., breast tissue sample; plasma sample; stool sample), e.g., a method comprising determining a methylation state of a marker in the sample comprising a base in a DMR that is one or more of DMR 1-375 (from Tables 2 and 5); comparing the methylation state of the marker from the subject sample to a methylation state of the marker from a normal control sample from a subject who does not have breast cancer; and determining a confidence interval and/or a p value of the difference in the methylation state of the subject sample and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001. Some embodiments of methods provide steps of reacting a nucleic acid comprising a DMR with a reagent capable of modifying nucleic acid in a methylation-specific manner (e.g., a methylation-sensitive restriction enzyme, a methylation-dependent restriction enzyme, and a bisulfite reagent) to produce, for example, nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have breast cancer and/or a form of breast cancer to identify differences in the two sequences; and identifying the subject as having breast cancer when a difference is present.

Systems for screening for breast cancer in a sample obtained from a subject are provided by the technology. Exemplary embodiments of systems include, e.g., a system for screening for breast cancer in a sample obtained from a subject (e.g., breast tissue sample; plasma sample; stool sample), the system comprising an analysis component configured to determine the methylation state of a sample, a software component configured to compare the methylation state of the sample with a control sample or a reference sample methylation state recorded in a database, and an alert component configured to alert a user of a breast-cancer-associated methylation state. An alert is determined in some embodiments by a software component that receives the results from multiple assays (e.g., determining the methylation states of multiple markers, e.g., DMR, e.g., as provided in Tables 2 and 5) and calculating a value or result to report based on the multiple results. Some embodiments provide a database of weighted parameters associated with each DMR provided herein for use in calculating a value or result and/or an alert to report to a user (e.g., such as a physician, nurse, clinician, etc.). In some embodiments all results from multiple assays are reported and in some embodiments one or more results are used to provide a score, value, or result based on a composite of one or more results from multiple assays that is indicative of a cancer risk in a subject.

In some embodiments of systems, a sample comprises a nucleic acid comprising a DMR. In some embodiments the system further comprises a component for isolating a nucleic acid, a component for collecting a sample such as a component for collecting a stool sample. In some embodiments, the system comprises nucleic acid sequences comprising a DMR. In some embodiments the database comprises nucleic acid sequences from subjects who do not have breast cancer. Also provided are nucleic acids, e.g., a set of nucleic acids, each nucleic acid having a sequence comprising a DMR. In some embodiments the set of nucleic acids wherein each nucleic acid has a sequence from a subject who does not have breast cancer and/or specific types of breast cancer. Related system embodiments comprise a set of nucleic acids as described and a database of nucleic acid sequences associated with the set of nucleic acids. Some embodiments further comprise a reagent capable of modifying DNA in a methylation-specific manner (e.g., a methylation-sensitive restriction enzyme, a methylation-dependent restriction enzyme, and a bisulfite reagent). And, some embodiments further comprise a nucleic acid sequencer.

In certain embodiments, methods for characterizing a sample (e.g., breast tissue sample; plasma sample; whole blood sample; serum sample; stool sample) from a human patient are provided. For example, in some embodiments such embodiments comprise obtaining DNA from a sample of a human patient; assaying a methylation state of a DNA methylation marker comprising a base in a differentially methylated region (DMR) selected from a group consisting of DMR 1-375 from Tables 2 and 5; and comparing the assayed methylation state of the one or more DNA methylation markers with methylation level references for the one or more DNA methylation markers for human patients not having breast cancer.

Such methods are not limited to a particular type of sample from a human patient. In some embodiments, the sample is a breast tissue sample. In some embodiments, the sample is a plasma sample. In some embodiments, the sample is a stool sample, a tissue sample, a breast tissue sample, a blood sample (e.g., plasma sample, whole blood sample, serum sample), or a urine sample.

In some embodiments, such methods comprise assaying a plurality of DNA methylation markers. In some embodiments, such methods comprise assaying 2 to 11 DNA methylation markers. In some embodiments, such methods comprise assaying 12 to 120 DNA methylation markers. In some embodiments, such methods comprise assaying 2 to 375 DNA methylation markers. In some embodiments, such methods comprise assaying the methylation state of the one or more DNA methylation markers in the sample comprises determining the methylation state of one base. In some embodiments, such methods comprise assaying the methylation state of the one or more DNA methylation markers in the sample comprises determining the extent of methylation at a plurality of bases. In some embodiments, such methods comprise assaying a methylation state of a forward strand or assaying a methylation state of a reverse strand.

In some embodiments, the DNA methylation marker is a region of 100 or fewer bases. In some embodiments, the DNA methylation marker is a region of 500 or fewer bases. In some embodiments, the DNA methylation marker is a region of 1000 or fewer bases. In some embodiments, the DNA methylation marker is a region of 5000 or fewer bases. In some embodiments, the DNA methylation marker is one base. In some embodiments, the DNA methylation marker is in a high CpG density promoter.

In some embodiments, the assaying comprises using methylation specific polymerase chain reaction, nucleic acid sequencing, mass spectrometry, methylation specific nuclease, mass-based separation, or target capture.

In some embodiments, the assaying comprises use of a methylation specific oligonucleotide. In some embodiments, the methylation specific oligonucleotide is selected from the group consisting of SEQ ID NO: 1-422 (Tables 3, 6, 7, 15 and 16).

In some embodiments, a chromosomal region having an annotation selected from the group consisting of ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23_D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1 (see, Table 4, Example II) comprises the DNA methylation marker.

In some embodiments, a chromosomal region having an annotation selected from the group consisting of ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B (see, Table 9, Example III) comprises the DNA methylation marker.

In some embodiments, a chromosomal region having an annotation selected from the group consisting of CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B (see, Table 14, Example III) comprises the DNA methylation marker.

In some embodiments, such methods comprise determining the methylation state of two DNA methylation markers. In some embodiments, such methods comprise determining the methylation state of a pair of DNA methylation markers provided in a row of Tables 2 and 5.

In certain embodiments, the technology provides methods for characterizing a sample (e.g., breast tissue sample; plasma sample; whole blood sample; serum sample; stool sample) obtained from a human patient. In some embodiments, such methods comprise determining a methylation state of a DNA methylation marker in the sample comprising a base in a DMR selected from a group consisting of DMR 1-375 from Tables 2 and 5; comparing the methylation state of the DNA methylation marker from the patient sample to a methylation state of the DNA methylation marker from a normal control sample from a human subject who does not have a breast cancer; and determining a confidence interval and/or a p value of the difference in the methylation state of the human patient and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001.

In certain embodiments, the technology provides methods for characterizing a sample obtained from a human subject (e.g., breast tissue sample; plasma sample; whole blood sample; serum sample; stool sample), the method comprising reacting a nucleic acid comprising a DMR with a reagent capable of modifying DNA in a methylation-specific manner (e.g., a methylation-sensitive restriction enzyme, a methylation-dependent restriction enzyme, and a bisulfite reagent) to produce nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have breast cancer to identify differences in the two sequences.

In certain embodiments, the technology provides systems for characterizing a sample obtained from a human subject (e.g., breast tissue sample; plasma sample; stool sample), the system comprising an analysis component configured to determine the methylation state of a sample, a software component configured to compare the methylation state of the sample with a control sample or a reference sample methylation state recorded in a database, and an alert component configured to determine a single value based on a combination of methylation states and alert a user of a breast cancer-associated methylation state. In some embodiments, the sample comprises a nucleic acid comprising a DMR.

In some embodiments, such systems further comprise a component for isolating a nucleic acid. In some embodiments, such systems further comprise a component for collecting a sample.

In some embodiments, the sample is a stool sample, a tissue sample, a breast tissue sample, a blood sample (e.g., plasma sample, whole blood sample, serum sample), or a urine sample.

In some embodiments, the database comprises nucleic acid sequences comprising a DMR. In some embodiments, the database comprises nucleic acid sequences from subjects who do not have a breast cancer.

Additional embodiments will be apparent to persons skilled in the relevant art based on the teachings contained herein.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93 (13) 9821-9826; herein incorporated by reference in its entirety), miniprimer PCR, multiplex ligation-dependent probe amplification (see, e.g., Schouten, et al., (2002) Nucleic Acids Research 30 (12): e57; herein incorporated by reference in its entirety), multiplex PCR (see, e.g., Chamberlain, et al., (1988) Nucleic Acids Research 16 (23) 11141-11156; Ballabio, et al., (1990) Human Genetics 84 (6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16 (15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S.A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19 (14) 4008; Roux, K. (1994) Biotechniques 16 (5) 812-814; Hecker, et al., (1996) Biotechniques 20 (3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic or other DNA or RNA, without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.” Those of skill in the art will understand the term “PCR” encompasses many variants of the originally described method using, e.g., real time PCR, nested PCR, reverse transcription PCR (RT-PCR), single primer and arbitrarily primed PCR, etc.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-beta replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al, Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics 4:560). Finally, thermostable template-dependant DNA polymerases (e.g., Taq and Pfu DNA polymerases), by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety).

The term “amplifiable nucleic acid” refers to a nucleic acid that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g, Antequera et al. (1990) Cell 62:503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196:261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97:5237-5242; Salmon and Kaye (1970) Biochim. Biophys. Acta. 204:340-351; Grafstrom (1985) Nucleic Acids Res. 13:2827-2842; Nyce (1986) Nucleic Acids Res. 14:4353-4367; Woodcock (1987) Biochem. Biophys. Res. Commun. 145:888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, and a bisulfite reagent.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

A change in the nucleic acid nucleotide sequence by a methylation-specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al. (1997) Cancer Research 57:594-599.

The term “MethyLight™” refers to the art-recognized fluorescence-based real-time PCR technique described by Eads et al. (1999) Cancer Res. 59:2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25:2529-2531.

The term “MSP” (Methylation-specific PCR) refers to the art-recognized methylation assay described by Herman et al. (1996) Proc. Natl. Acad. Sci. USA 93:9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25:2532-2534.

The term “MCA” (Methylated CpG Island Amplification) refers to the methylation assay described by Toyota et al. (1999) Cancer Res. 59:2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant.

The term “pre-cancerous” or “pre-neoplastic” and equivalents thereof refer to any cellular proliferative disorder that is undergoing malignant transformation.

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, by be combined with other nucleic acids or molecules. For example, an isolated nucleic acid may be present in a host cell in which into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, a “remote sample” as used in some contexts relates to a sample indirectly collected from a site that is not the cell, tissue, or organ source of the sample. For instance, when sample material originating from the pancreas is assessed in a stool sample (e.g., not from a sample taken directly from a breast), the sample is a remote sample.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including race horses), and the like. The presently-disclosed subject matter further includes a system for diagnosing a lung cancer in a subject. The system can be provided, for example, as a commercial kit that can be used to screen for a risk of lung cancer or diagnose a lung cancer in a subject from whom a biological sample has been collected. An exemplary system provided in accordance with the present technology includes assessing the methylation state of a marker described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520 (e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “breast cancer” refers generally to the uncontrolled growth of breast tissue and, more specifically, to a condition characterized by anomalous rapid proliferation of abnormal cells in one or both breasts of a subject. The abnormal cells often are referred to as malignant or “neoplastic cells,” which are transformed cells that can form a solid tumor. The term “tumor” refers to an abnormal mass or population of cells (i.e., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### DETAILED DESCRIPTION

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

Provided herein is technology for breast cancer screening and particularly, but not exclusively, to methods, compositions, and related uses for detecting the presence of breast cancer. As the technology is described herein, the section headings used are for organizational purposes only and are not to be construed as limiting the subject matter in any way.

Indeed, as described in Examples I, II and III, experiments conducted during the course for identifying embodiments for the present invention identified a novel set of 375 differentially methylated regions (DMRs) for discriminating cancer of the breast derived DNA from non-neoplastic control DNA. In addition, DMRs were identified capable of plasma from subjects having breast cancer from plasma from subjects not having breast cancer.

Although the disclosure herein refers to certain illustrated embodiments, it is to be understood that these embodiments are presented by way of example and not by way of limitation.

In particular aspects, the present technology provides compositions and methods for identifying, determining, and/or classifying a cancer such as breast cancer. The methods comprise determining the methylation status of at least one methylation marker in a biological sample isolated from a subject (e.g., stool sample, breast tissue sample, plasma sample), wherein a change in the methylation state of the marker is indicative of the presence, class, or site of a breast cancer. Particular embodiments relate to markers comprising a differentially methylated region (DMR, e.g., DMR 1-375, see Tables 2 and 5) that are used for diagnosis (e.g., screening) of breast cancer.

In addition to embodiments wherein the methylation analysis of at least one marker, a region of a marker, or a base of a marker comprising a DMR (e.g., DMR, e.g., DMR 1-375) provided herein and listed in Tables 2 and 5 is analyzed, the technology also provides panels of markers comprising at least one marker, region of a marker, or base of a marker comprising a DMR with utility for the detection of cancers, in particular breast cancer.

Some embodiments of the technology are based upon the analysis of the CpG methylation status of at least one marker, region of a marker, or base of a marker comprising a DMR.

In some embodiments, the present technology provides for the use of a reagent that modifies DNA in a methylation-specific manner (e.g., a methylation-sensitive restriction enzyme, a methylation-dependent restriction enzyme, and a bisulfite reagent) in combination with one or more methylation assays to determine the methylation status of CpG dinucleotide sequences within at least one marker comprising a DMR (e.g., DMR 1-375, see Tables 2 and 5). Genomic CpG dinucleotides can be methylated or unmethylated (alternatively known as up- and down-methylated respectively). However the methods of the present invention are suitable for the analysis of biological samples of a heterogeneous nature, e.g., a low concentration of tumor cells, or biological materials therefrom, within a background of a remote sample (e.g., blood, organ effluent, or stool). Accordingly, when analyzing the methylation status of a CpG position within such a sample one may use a quantitative assay for determining the level (e.g., percent, fraction, ratio, proportion, or degree) of methylation at a particular CpG position.

According to the present technology, determination of the methylation status of CpG dinucleotide sequences in markers comprising a DMR has utility both in the diagnosis and characterization of cancers such as breast cancer.

### Combinations of Markers

In some embodiments, the technology relates to assessing the methylation state of combinations of markers comprising a DMR from Tables 2 and 5 (e.g., DMR Nos. 1-375). In some embodiments, assessing the methylation state of more than one marker increases the specificity and/or sensitivity of a screen or diagnostic for identifying a neoplasm in a subject (e.g., breast cancer).

Various cancers are predicted by various combinations of markers, e.g., as identified by statistical techniques related to specificity and sensitivity of prediction. The technology provides methods for identifying predictive combinations and validated predictive combinations for some cancers.

### Methods for Assaying Methylation State

In certain embodiments, methods for analyzing a nucleic acid for the presence of 5-methylcytosine involves treatment of DNA with a reagent that modifies DNA in a methylation-specific manner. Examples of such reagents include, but are not limited to, a methylation-sensitive restriction enzyme, a methylation-dependent restriction enzyme, and a bisulfite reagent.

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89:1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16:431-36; Grigg G, DNA Seq. (1996) 6:189-98), methylation-specific PCR (MSP) as is disclosed, e.g., in U.S. U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24:5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26:2255.

The bisulfite technique typically involves amplifying short, specific fragments of a known nucleic acid subsequent to a bisulfite treatment, then either assaying the product by sequencing (Olek & Walter (1997) Nat. Genet. 17:275-6) or a primer extension reaction (Gonzalgo & Jones (1997) Nucleic Acids Res. 25:2529-31; WO 95/00669; U.S. Pat. No. 6,251,594) to analyze individual cytosine positions. Some methods use enzymatic digestion (Xiong & Laird (1997) Nucleic Acids Res. 25:2532-4). Detection by hybridization has also been described in the art (Olek et al., WO 99/28498). Additionally, use of the bisulfite technique for methylation detection with respect to individual genes has been described (Grigg & Clark (1994) Bioessays 16:431-6,; Zeschnigk et al. (1997) Hum Mol Genet. 6:387-95; Feil et al. (1994) Nucleic Acids Res. 22:695; Martin et al. (1995) Gene 157:261-4; WO 9746705; WO 9515373).

Various methylation assay procedures can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89:1827-1831). Additionally, restriction enzyme digestion of PCR products amplified from bisulfite-converted DNA finds use in assessing methylation state, e.g., as described by Sadri & Hornsby (1997) Nucl. Acids Res. 24:5058-5059 or as embodied in the method known as COBRA (Combined Bisulfite Restriction Analysis) (Xiong & Laird (1997) Nucleic Acids Res. 25:2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components. Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7:133-6; Meissner et al. (2005) “Reduced representation bisulfite sequencing for comparative high-resolution DNA methylation analysis” Nucleic Acids Res. 33:5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophore per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides according to the present invention (e.g., see Tables 3, 6, 7, 15 and 16) and an amplification enzyme. The amplification of several DNA segments can be carried out simultaneously in one and the same reaction vessel. Typically, the amplification is carried out using a polymerase chain reaction (PCR). Amplicons are typically 100 to 2000 base pairs in length.

In another embodiment of the method, the methylation status of CpG positions within or near a marker comprising a DMR (e.g., DMR 1-375, Tables 2 and 5) may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer. Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances compromise the accuracy and precision of these assays directed to detecting such low amounts of a target. Accordingly, provided herein are methods providing the requisite management of volume and concentration to produce such DNA samples.

In some embodiments, the sample comprises blood, serum, plasma, or saliva. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events. The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

It is contemplated that embodiments of the technology are provided in the form of a kit. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

### Methods

In some embodiments of the technology, methods are provided that comprise the following steps:
1) contacting a nucleic acid (e.g., genomic DNA, e.g., isolated from body fluids such as blood or plasma or breast tissue) obtained from the subject with at least one reagent or series of reagents that distinguishes between methylated and non-methylated CpG dinucleotides within at least one marker comprising a DMR (e.g., DMR 1-375 e.g., as provided in Tables 2 and 5) and
2) detecting breast cancer (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma or breast tissue) obtained from the subject with at least one reagent or series of reagents that distinguishes between methylated and non-methylated CpG dinucleotides within at least one marker selected from a chromosomal region having an annotation selected from the group consisting of ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23_D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1, and
2) detecting breast cancer (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma or breast tissue) obtained from the subject with at least one reagent or series of reagents that distinguishes between methylated and non-methylated CpG dinucleotides within at least one marker selected from a chromosomal region having an annotation selected from the group consisting of ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, KLHDC7B_B, LAYN_B, LIME1_B, LMX1B_D, LOC100132891_B, MAST1_B, MAX.chr12.427.br, MAX.chr17.73073682-73073814, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TBX1_B, TRH_A, and TRIM67_B, and
2) detecting breast cancer (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from body fluids such as blood or plasma or breast tissue) obtained from the subject with at least one reagent or series of reagents that distinguishes between methylated and non-methylated CpG dinucleotides within at least one marker selected from a chromosomal region having an annotation selected from the group consisting of CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B, and
2) detecting breast cancer (e.g., afforded with a sensitivity of greater than or equal to 80% and a specificity of greater than or equal to 80%).

In some embodiments of the technology, methods are provided that comprise the following steps:
1) measuring a methylation level for one or more genes in a biological sample of a human individual through treating genomic DNA in the biological sample with a reagent that modifies DNA in a methylation-specific manner (e.g., wherein the reagent is a bisulfite reagent, a methylation-sensitive restriction enzyme, or a methylation-dependent restriction enzyme), wherein the one or more genes is selected from one of the following groups:
   (i) ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23_D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1;
   (ii) ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B; and
   (iii) CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B;
2) amplifying the treated genomic DNA using a set of primers for the selected one or more genes; and
3) determining the methylation level of the one or more genes by polymerase chain reaction, nucleic acid sequencing, mass spectrometry, methylation-specific nuclease, mass-based separation, and target capture.

In some embodiments of the technology, methods are provided that comprise the following steps:
1) measuring an amount of at least one methylated marker gene in DNA from the sample, wherein the one or more genes is selected from one of the following groups:
   (i) ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8. 124173030-124173395, MAX.chr20.1784209-1784461, LOC100132891, BHLHE23_D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1;
   (ii) ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B; and
   (iii) CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B;
2) measuring the amount of at least one reference marker in the DNA; and
3) calculating a value for the amount of the at least one methylated marker gene measured in the DNA as a percentage of the amount of the reference marker gene measured in the DNA, wherein the value indicates the amount of the at least one methylated marker DNA measured in the sample.

In some embodiments of the technology, methods are provided that comprise the following steps:
1) measuring a methylation level of a CpG site for one or more genes in a biological sample of a human individual through treating genomic DNA in the biological sample with bisulfite a reagent capable of modifying DNA in a methylation-specific manner (e.g., a methylation-sensitive restriction enzyme, a methylation-dependent restriction enzyme, and a bisulfite reagent);
2) amplifying the modified genomic DNA using a set of primers for the selected one or more genes; and
3) determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR; wherein the one or more genes is selected from one of the following groups:
   (i) ATP6V1B1, LMX1B_A, BANK1, OTX1, MAX.chr11.14926602-14927148, UBTF, PRKCB, TRH_A, MPZ, DNM3_A, TRIM67, MAX.chr12.4273906-4274012, CALN1_A, ITPRIPL1, MAX.chr12.4273906-4274012, GYPC_B, MAX.chr5.42994866-42994936, OSR2_A, SCRT2_B, MAX.chr5.145725410-145725459, MAX.chr11.68622869-68622968, MAX.chr8.124173030-124173395, MAX.chr20.1784209-1784461, LOC100132891, BHLHE23_D, MAX.chr19.46379903-46380197, CHST2_B, MAX.chr5.77268672-77268725, C17orf64, EMX1_A, DSCR6, ITPRIPL1, IGF2BP3_B, DLX4, and ABLIM1;
   (ii) ABLIM1_B, AJAP1_C, ALOX5_B, ASCL2_B, BANK1_B, BHLHE23_E, C10orf125_B, C17orf64_B, CALN1_1520, CALN1_B, CD1D_1058, CDH4_7890, CHST2_8128, CHST2_8384, CHST2_9316, CHST2_9470, CLIC6_B, CXCL12_B, DLX4_B, DNM3_D, EMX1_A, ESPN_B, FAM59B_7764, FOXP4_B, GP5, HOXA1_C, IGF2BP3_C, IPTRIPL1_1138, IPTRIPL1_1200, KCNK9_B, KCNK17_C, LAYN_B, LIME1_B, LMX1B_D, LOC100132891_B, MAST1_B, MAX.chr12.427.br, MAX.chr20.4422, MPZ_5742, MPZ_5554, MSX2P1_B, ODC1_B, OSR2_A, OTX1_B, PLXNC1_B, PRKCB_7570, SCRT2_C, SLC30A10, SPHK2_B, ST8SIA4_B, STX16_C, TRH_A, and TRIM67_B; and
   (iii) CD1D, ITPRIPL1, FAM59B, C10orf125, TRIM67, SPHK2, CALN1_B, CHST2_B, MPZ, CXCL12_B, ODC1_B, OSR2_A, TRH_A, and C17orf64_B.

Preferably, the sensitivity for such methods is from about 70% to about 100%, or from about 80% to about 90%, or from about 80% to about 85%. Preferably, the specificity is from about 70% to about 100%, or from about 80% to about 90%, or from about 80% to about 85%.

Genomic DNA may be isolated by any means, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, breast tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, that distinguishes between methylated and non-methylated CpG dinucleotides within at least one marker comprising a DMR (e.g., DMR 1-375, e.g., as provided by Tables 2 and 5).

In some embodiments, the reagent converts cytosine bases which are unmethylated at the 5′-position to uracil, thymine, or another base which is dissimilar to cytosine in terms of hybridization behavior. However in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from DMR 1-375, e.g., as provided in Tables 2 and 5). The method of analysis may be selected from those known in the art, including those listed herein, e.g., QuARTS and MSP as described herein.

Aberrant methylation, more specifically hypermethylation of a marker comprising a DMR (e.g., DMR 1-375, e.g., as provided by Tables 2 and 5) is associated with a breast cancer.

The technology relates to the analysis of any sample associated with a breast cancer. For example, in some embodiments the sample comprises a tissue and/or biological fluid obtained from a patient. In some embodiments, the sample comprises a secretion. In some embodiments, the sample comprises blood, serum, plasma, gastric secretions, pancreatic juice, a gastrointestinal biopsy sample, microdissected cells from a breast biopsy, and/or cells recovered from stool. In some embodiments, the sample comprises breast tissue. In some embodiments, the subject is human. The sample may include cells, secretions, or tissues from the breast, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, pancreatic fluid, fluid obtained during endoscopy, blood, mucus, or saliva. In some embodiments, the sample is a stool sample. In some embodiments, the sample is a breast tissue sample.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens

In some embodiments, the technology relates to a method for treating a patient (e.g., a patient with breast cancer, with early stage breast cancer, or who may develop breast cancer), the method comprising determining the methylation state of one or more DMR as provided herein and administering a treatment to the patient based on the results of determining the methylation state. The treatment may be administration of a pharmaceutical compound, a vaccine, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments of the technology, a method for diagnosing a breast cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a biomarker (e.g., a DMR as disclosed herein), the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of breast cancer, and/or monitor the efficacy of appropriate therapies directed against the cancer. In such an embodiment for example, one might expect to see a change in the methylation state of one or more biomarkers (e.g., DMR) disclosed herein (and potentially one or more additional biomarker(s), if monitored) in a biological sample over time during the course of an effective therapy.

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with breast cancer risk, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR), the chance of a given outcome (e.g., suffering from a breast cancer) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR) can be established, and the degree of change in the methylation state of the biamarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in tissue taken from donors with metaplasia or from donors with a breast cancer. In certain embodiments of the method, a subject is identified as having metaplasia upon identifying an aberrant methylation state of one or more DMR provided herein in a biological sample obtained from the subject. In other embodiments of the method, the detection of an aberrant methylation state of one or more of such biomarkers in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having a breast cancer if, when compared to a control methylation state, there is a measurable difference in the methylation state of at least one biomarker in the sample. Conversely, when no change in methylation state is identified in the biological sample, the subject can be identified as not having breast cancer, not being at risk for the cancer, or as having a low risk of the cancer. In this regard, subjects having the cancer or risk thereof can be differentiated from subjects having low to substantially no cancer or risk thereof. Those subjects having a risk of developing a breast cancer can be placed on a more intensive and/or regular screening schedule, including endoscopic surveillance. On the other hand, those subjects having low to substantially no risk may avoid being subjected to additional testing for breast cancer (e.g., invasive procedure), until such time as a future screening, for example, a screening conducted in accordance with the present technology, indicates that a risk of breast cancer has appeared in those subjects.

As mentioned above, depending on the embodiment of the method of the present technology, detecting a change in methylation state of the one or more biomarkers can be a qualitative determination or it can be a quantitative determination. As such, the step of diagnosing a subject as having, or at risk of developing, a breast cancer indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specifically state or range of state. As such, the predetermined methylation state can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including race horses), and the like.

The presently-disclosed subject matter further includes a system for diagnosing a breast cancer in a subject. The system can be provided, for example, as a commercial kit that can be used to screen for a risk of breast cancer or diagnose a breast cancer in a subject from whom a biological sample has been collected. An exemplary system provided in accordance with the present technology includes assessing the methylation state of a DMR as provided in Tables 2 and 5.

### EXAMPLES

### Example I

This example describes the discovery and tissue validation of breast-cancer specific markers.

Table 1 shows the number of tissue samples for each subtype of breast cancer used in the discovery of breast cancer specific markers.

TABLE 1

| | Number of | |
|---|---|---|
| Breast Cancer Subtype | Subjects | Total |
| Basal-like/Triple Negative | 18 | 18 |
| HER2⁺ | 18 | 18 |
| Luminal A | 18 | 18 |
| Luminal B | 18 | 18 |
| BRCA 1 | 6 | 15 |
| BRCA 2 | 9 | |
| Normal Breast | 18 | 45 |
| Normal Breast + BRCA | 9 | |
| Normal Buffy Coat | 18 | |

For discovery of methylation markers by RRBS, frozen tissue samples were obtained from 72 invasive breast cancer cases (18 luminal A, 18 luminal B, 18 basal-like/triple negative, and 18 HER2+), 15 invasive breast cancer from BRCA germline mutation patients (6 BRCA1, 9 BRCA2), and 45 controls (18 normal breast (reduction mammoplasty or prophylactic mastectomy, 9 histologically normal breast in germline BRCA carriers (prophylactic mastectomy), and 18 normal buffy coat)). Tumor and breast tissue sections were reviewed by an expert GI pathologist to confirm diagnosis and estimate abnormal cellularity. Sections were then macro-dissected. Genomic DNA was purified using the QiaAmp Mini kit (Qiagen, Valencia CA). DNA (300 ng) was fragmented by digestion with 10 Units of MspI. Digested fragments were end-repaired and A-tailed with 5 Units of Klenow fragment (3′-5′ exo-), and ligated overnight to methylated TruSeq adapters (Illumina, San Diego CA) containing barcode sequences (to link each fragment to its sample ID.) Reactions were purified using AMPure XP SPRI beads/buffer (Beckman Coulter, Brea CA).

Tissue samples then underwent bisulfite conversion (twice) using a modified EpiTect protocol (Qiagen). qPCR (LightCycler 480-Roche, Mannheim Germany) was used to determine the optimal enrichment Ct. The following conditions were used for final enrichment PCR: Each 50 uL reaction contained 5 μL of 10× buffer, 1.25 uL of 10 mM each deoxyribonucleotide triphosphate (dNTP), 5 uL primer cocktail (˜5 uM), 15 uL template (sample), 1 uL PfuTurbo Cx hotstart (Agilent, Santa Clara CA) and 22.75 water; temperatures and times were 95C-5 min; 98C-30 sec; 16 cycles of 98C-10 sec, 65C-30 sec, 72C-30 sec, 72C-5 min and 4C hold, respectively. Samples were SPRI bead purified and then tested on the Bioanalyzer 2100 (Agilent) to assess the DNA size distribution of the enrichment. Size selection of 160-520 bp fragments (40-400 bp inserts) was performed using AMPure XP SPRI beads/buffer (Beckman Coulter, Brea CA). Buffer cutoffs were 0.7×-1.1× sample volumes. Samples were combined (equimolar) into 4-plex libraries based on the randomization scheme and tested with the bioanalyzer for final size and concentration verification, and with qPCR (KAPA Library Quantification Kit-KAPA Biosystems, Cape Town South Africa).

Tissue samples were loaded onto single read flow cells according to a randomized lane assignment and sequencing was performed by the Next Generation Sequencing Core at the Mayo Clinic Medical Genome Facility on the Illumina HiSeq 2000 platform. Reads were unidirectional for 101 cycles. The standard Illumina pipeline was run for the primary analysis. SAAP-RRBS (streamlined analysis and annotation pipeline for reduced representation bisulfite sequencing) was used for quality scoring, sequence alignment, annotation, and methylation extraction.

Breast cancer tissue yielded large numbers of discriminate DMRs, many of which had not been identified before. Comparing the methylation of breast cancer tissue samples to normal breast tissue, 327 methylated regions were identified (see, Table 2) that distinguished breast cancer tissue from normal breast tissue (the genomic coordinates for the regions shown in Table 2 are based on the Human February 2009 (GRCh37/hg19) Assembly).

TABLE 2
Identified methylated regions distinguishing
breast cancer tissue from normal breast tissue.

| DMR | | Region on Chromosome |
|---|---|---|
| No. | Gene Annotation | (starting base-ending base) |
| 1 | ZSCAN23 | chr6: 28411152-28411272 |
| 2 | AADAT.R | chr4: 171010951-171010991 |
| 3 | ABLIM1 | chr10: 116391588-116391793 |
| 4 | ACCN1 | chr17: 31620207-31620314 |
| 5 | AFAP1L1 | chr5: 148651161-148651242 |
| 6 | AJAP1_A | chr1: 4715535-4715646 |
| 7 | AJAP1_B | chr1: 4715931-4716021 |
| 8 | AKR1B1 | chr7: 134143171-134143684 |
| 9 | ALOX5 | chr10: 45914840-45914949 |
| 10 | AMN | chr14: 103394920-103395019 |
| 11 | ANPEP | chr15: 90358420-90358514 |
| 12 | ANTXR2 | chr4: 80993475-80993634 |
| 13 | ARL5C | chr17: 37321515-37321626 |
| 14 | ASCL2 | chr11: 2292240-2292361 |
| 15 | ATP6V1B1 | chr2: 71192354-71192453 |
| 16 | B3GNT5 | chr3: 182971589-182971825 |
| 17 | BANK1 | chr4: 102711871-102712076 |
| 18 | BCAT1 | chr12: 25055906-25055975 |
| 19 | BEGAIN | chr14: 101033665-101033813 |
| 20 | BEST4 | chr1: 45251853-45252029 |
| 21 | BHLHE23_A | chr20: 61637950-61637986 |
| 22 | BHLHE23_B | chr20: 61638020-61638083 |
| 23 | BHLHE23_C | chr20: 61638088-61638565 |
| 24 | BHLHE23_D | chr20: 61638244-61638301 |
| 25 | BMP4 | chr14: 54421578-54421916 |
| 26 | BMP6 | chr6: 7727566-7727907 |
| 27 | C10orf125 | chr10: 135171410-135171504 |
| 28 | C10orf93 | chr10: 134756078-134756167 |
| 29 | C17orf64 | chr17: 58499095-58499190 |
| 30 | C19orf35 | chr19: 2282568-2282640 |
| 31 | C19orf66 | chr19: 10197688-10197823 |
| 32 | C1QL2 | chr2: 119916511-119916572 |
| 33 | C20orf195_A | chr20: 62185293-62185364 |
| 34 | C20orf195_B | chr20: 62185418-62185546 |
| 35 | C7orf52 | chr7: 100823483-100823514 |
| 36 | CALN1_A | chr7: 71801486-71801594 |
| 37 | CALN1_B | chr7: 71801741-71801800 |
| 38 | CAMKV | chr3: 49907259-49907298 |
| 39 | CAPN2.FR | chr1: 223900347-223900405 |
| 40 | CAV2 | chr7: 116140205-116140342 |
| 41 | CBLN1_A | chr16: 49315588-49315691 |
| 42 | CBLN1_B | chr16: 49316198-49316258 |
| 43 | CCDC61 | chr19: 46519467-46519536 |
| 44 | CCND2_A | chr12: 4378317-4378375 |
| 45 | CCND2_B | chr12: 4380560-4380681 |
| 46 | CCND2_C | chr12: 4384096-4384146 |
| 47 | CD1D | chr1: 158150864-158151129 |
| 48 | CD8A | chr2: 87017780-87017917 |
| 49 | CDH4_A | chr20: 59827230-59827285 |
| 50 | CDH4_B | chr20: 59827762-59827776 |
| 51 | CDH4_C | chr20: 59827794-59827868 |
| 52 | CDH4_D | chr20: 59828193-59828258 |
| 53 | CDH4_E | chr20: 59828479-59828729 |
| 54 | CDH4_F | chr20: 59828778-59828814 |
| 55 | CHRNA7 | chr15: 32322830-32322897 |
| 56 | CHST2_A | chr3: 142838025-142838494 |
| 57 | CHST2_B | chr3: 142839223-142839568 |
| 58 | CLIC6 | chr21: 36042025-36042131 |
| 59 | CLIP4 | chr2: 29338109-29338339 |
| 60 | COL23A1.R | chr5: 178017669-178017854 |
| 61 | CR1 | chr1: 207669481-207669639 |
| 62 | CRHBP | chr5: 76249939-76249997 |
| 63 | CXCL12.F | chr10: 44881210-44881300 |
| 64 | DBNDD1.FR | chr16: 90085625-90085681 |
| 65 | DLK1 | chr14: 101193295-101193318 |
| 66 | DLX4 | chr17: 48042562-48042606 |
| 67 | DLX6 | chr7: 96635255-96635475 |
| 68 | DNAJC6 | chr1: 65731412-65731507 |
| 69 | DNM3_A | chr1: 171810393-171810575 |
| 70 | DNM3_B | chr1: 171810648-171810702 |
| 71 | DNM3_C | chr1: 171810806-171810920 |
| 72 | DSCR6 | chr21: 38378540-38378601 |
| 73 | DTX1 | chr12: 113515535-113515637 |
| 74 | EMX1_A | chr2: 73151498-73151578 |
| 75 | EMX1_B | chr2: 73151663-73151756 |
| 76 | EPHA4 | chr2: 222436217-222436320 |
| 77 | ESPN | chr1: 6508784-6509175 |
| 78 | ESYT3 | chr3: 138153979-138154071 |
| 79 | ETS1_A | chr11: 128391809-128391908 |
| 80 | ETS1_B | chr11: 128392062-128392309 |
| 81 | FABP5 | chr8: 82192605-82192921 |
| 82 | FAIM2 | chr12: 50297863-50297988 |
| 83 | FAM126A | chr7: 23053941-23054066 |
| 84 | FAM129C.F | chr19: 17650551-17650610 |
| 85 | FAM150A | chr8: 53478266-53478416 |
| 86 | FAM150B | chr2: 287868-287919 |
| 87 | FAM171A1 | chr10: 15412558-15412652 |
| 88 | FAM189A1 | chr15: 29862130-29862169 |
| 89 | FAM20A | chr17: 66597237-66597326 |
| 90 | FAM59B | chr2: 26407713-26407972 |
| 91 | FBN1 | chr15: 48937412-48937541 |
| 92 | FLJ42875 | chr1: 2987037-2987116 |
| 93 | FLRT2 | chr14: 85998469-85998535 |
| 94 | FMN2 | chr1: 240255171-240255253 |
| 95 | FMNL2 | chr2: 153192734-153192836 |
| 96 | FOXP4 | chr6: 41528816-41528958 |
| 97 | FSCN1 | chr7: 5633506-5633615 |
| 98 | GAD2 | chr10: 26505066-26505385 |
| 99 | GAS7 | chr17: 10101325-10101397 |
| 100 | GCGR | chr17: 79761970-79762088 |
| 101 | GLI3 | chr7: 42267808-42267899 |
| 102 | GLP1R | chr6: 39016381-39016421 |
| 103 | GNG4 | chr1: 235813658-235813798 |
| 104 | GP5 | chr3: 194118738-194118924 |
| 105 | GRASP | chr12: 52400919-52401166 |
| 106 | GRM7 | chr3: 6902873-6902931 |
| 107 | GSTP1 | chr11: 67350986-67351055 |
| 108 | GYPC_A | chr2: 127413505-127413678 |
| 109 | GYPC_B | chr2: 127414096-127414189 |
| 110 | HAND2 | chr4: 174450452-174450478 |
| 111 | HBM | chr16: 216426-216451 |
| 112 | HES5 | chr1: 2461823-2461915 |
| 113 | HHEX.F | chr10: 94449486-94449597 |
| 114 | HMGA2 | chr12: 66219385-66219487 |
| 115 | HNF1B_A | chr17: 36103713-36103793 |
| 116 | HNF1B_B | chr17: 36105390-36105448 |
| 117 | HOXA1_A | chr7: 27135603-27135889 |
| 118 | HOXA1_B | chr7: 27136191-27136244 |
| 119 | HOXA7_A | chr7: 27195742-27195895 |
| 120 | HOXA7_B | chr7: 27196032-27196190 |
| 121 | HOXA7_C | chr7: 27196441-27196531 |
| 122 | HOXD9 | chr2: 176987716-176987739 |
| 123 | IGF2BP3_A | chr7: 23508901-23509225 |
| 124 | IGF2BP3_B | chr7: 23513817-23514114 |
| 125 | IGFBP5 | chr2: 217559103-217559244 |
| 126 | IGSF9B_A | chr11: 133825409-133825476 |
| 127 | IGSF9B_B | chr11: 133825491-133825530 |
| 128 | IL15RA | chr10: 6018610-6018848 |
| 129 | IL17REL | chr22: 50453462-50453555 |
| 130 | INSM1 | chr20: 20348140-20348182 |
| 131 | ITGA9 | chr3: 37493895-37493994 |
| 132 | ITPKA_A | chr15: 41787438-41787784 |
| 133 | ITPKA_B | chr15: 41793928-41794003 |
| 134 | ITPRIPL1 | chr2: 96990968-96991328 |
| 135 | JSRP1 | chr19: 2253163-2253376 |
| 136 | KCNA1 | chr12: 5019401-5019633 |
| 137 | KCNE3 | chr11: 74178260-74178346 |
| 138 | KCNH8 | chr3: 19189837-19189897 |
| 139 | KCNK17_A | chr6: 39281195-39281282 |
| 140 | KCNK17_B | chr6: 39281408-39281478 |
| 141 | KCNK9.FR | chr8: 140715096-140715164 |
| 142 | KCNQ2 | chr20: 62103558-62103625 |
| 143 | KIAA1949 | chr6: 30646976-30647084 |
| 144 | KIRREL2 | chr19: 36347825-36347863 |
| 145 | KLF16 | chr19: 1857330-1857476 |
| 146 | KLHDC7B | chr22: 50987219-50987304 |
| 147 | LAYN.R | chr11: 111412023-111412074 |
| 148 | LIME1 | chr20: 62369116-62369393 |
| 149 | LMX1B_A | chr9: 129388175-129388223 |
| 150 | LMX1B_B | chr9: 129388231-129388495 |
| 151 | LMX1B_C | chr9: 129445588-129445603 |
| 152 | LOC100131176 | chr7: 151106986-151107060 |
| 153 | LOC100132891 | chr8: 72755897-72756295 |
| 154 | LOC100302401.R | chr1: 178063509-178063567 |
| 155 | LOC283999 | chr17: 76227905-76227960 |
| 156 | LRRC34 | chr3: 169530006-169530139 |
| 157 | LSS.F | chr21: 47649525-47649615 |
| 158 | LY6H | chr8: 144241547-144241557 |
| 159 | MAGI2 | chr7: 79083359-79083600 |
| 160 | MAST1 | chr19: 12978399-12978642 |
| 161 | MAX.chr1.158083198-158083476 | chr1: 158083198-158083476 |
| 162 | MAX.chr1.228074764-228074977 | chr1: 228074764-228074977 |
| 163 | MAX.chr1.239549742-239549886 | chr1: 239549742-239549886 |
| 164 | MAX.chr1.46913931-46913950 | chr1: 46913931-46913950 |
| 165 | MAX.chr1.8277285-8277316 | chr1: 8277285-8277316 |
| 166 | MAX.chr1.8277479-8277527 | chr1: 8277479-8277527 |
| 167 | MAX.chr10.130085265-130085312 | chr10: 130085265-130085312 |
| 168 | MAX.chr11.14926602-14927148 | chr11: 14926602-14927148 |
| 169 | MAX.chr11.68622869-68622968 | chr11: 68622869-68622968 |
| 170 | MAX.chr12.4273906-4274012 | chr12: 4273906-4274012 |
| 171 | MAX.chr12.59990591-59990895 | chr12: 59990591-59990895 |
| 172 | MAX.chr14.101176106-101176260 | chr14: 101176106-101176260 |
| 173 | MAX.chr15.96889013-96889128 | chr15: 96889013-96889128 |
| 174 | MAX.chr17.73073682-73073814 | chr17: 73073682-73073814 |
| 175 | MAX.chr17.8230197-8230314 | chr17: 8230197-8230314 |
| 176 | MAX.chr18.5629721-5629791 | chr18: 5629721-5629791 |
| 177 | MAX.chr18.76734362-76734476 | chr18: 76734362-76734476 |
| 178 | MAX.chr19.30719261-30719354 | chr19: 30719261-30719354 |
| 179 | MAX.chr19.46379903-46380197 | chr19: 46379903-46380197 |
| 180 | MAX.chr2.223183057-223183114.FR | chr2: 223183057-223183114 |
| 181 | MAX.chr2.238864674-238864735 | chr2: 238864674-238864735 |
| 182 | MAX.chr2.97193163-97193287 | chr2: 97193163-97193287 |
| 183 | MAX.chr2.97193478-97193562 | chr2: 97193478-97193562 |
| 184 | MAX.chr20.1783841-1784054 | chr20: 1783841-1784054 |
| 185 | MAX.chr20.1784209-1784461 | chr20: 1784209-1784461 |
| 186 | MAX.chr21.44782441-44782498 | chr21: 44782441-44782498 |
| 187 | MAX.chr21.47063802-47063851 | chr21: 47063802-47063851 |
| 188 | MAX.chr22.23908718-23908782 | chr22: 23908718-23908782 |
| 189 | MAX.chr22.42679578-42679917 | chr22: 42679578-42679917 |
| 190 | MAX.chr4.8859253-8859329 | chr4: 8859253-8859329 |
| 191 | MAX.chr4.8859602-8859669 | chr4: 8859602-8859669 |
| 192 | MAX.chr4.8860002-8860038 | chr4: 8860002-8860038 |
| 193 | MAX.chr5.145725410-145725459 | chr5: 145725410-145725459 |
| 194 | MAX.chr5.172234248-172234494 | chr5: 172234248-172234494 |
| 195 | MAX.chr5.178957564-178957598 | chr5: 178957564-178957598 |
| 196 | MAX.chr5.180101084-180101094 | chr5: 180101084-180101094 |
| 197 | MAX.chr5.42952185-42952280 | chr5: 42952185-42952280 |
| 198 | MAX.chr5.42994866-42994936 | chr5: 42994866-42994936 |
| 199 | MAX.chr5.77268672-77268725 | chr5: 77268672-77268725 |
| 200 | MAX.chr5.81148300-81148332 | chr5: 81148300-81148332 |
| 201 | MAX.chr6.108440684-108440788 | chr6: 108440684-108440788 |
| 202 | MAX.chr6.130686865-130686985 | chr6: 130686865-130686985 |
| 203 | MAX.chr6.157556793-157556856 | chr6: 157556793-157556856 |
| 204 | MAX.chr6.157557371-157557657 | chr6: 157557371-157557657 |
| 205 | MAX.chr6.27064703-27064783 | chr6: 27064703-27064783 |
| 206 | MAX.chr7.151145632-151145743 | chr7: 151145632-151145743 |
| 207 | MAX.chr7.152622607-152622638 | chr7: 152622607-152622638 |
| 208 | MAX.chr8.124173030-124173395 | chr8: 124173030-124173395 |
| 209 | MAX.chr8.124173128-124173268 | chr8: 124173128-124173268 |
| 210 | MAX.chr8.143533298-143533558 | chr8: 143533298-143533558 |
| 211 | MAX.chr8.145104132-145104218 | chr8: 145104132-145104218 |
| 212 | MAX.chr8.687688-687736 | chr8: 687688-687736 |
| 213 | MAX.chr8.688863-688924 | chr8: 688863-688924 |
| 214 | MAX.chr9.114010-114207 | chr9: 114010-114207 |
| 215 | MAX.chr9.136474504-136474527 | chr9: 136474504-136474527 |
| 216 | MCF2L2 | chr3: 182896930-182897245 |
| 217 | MERTK | chr2: 112656676-112656744 |
| 218 | MGAT1 | chr5: 180230434-180230767 |
| 219 | MIB2 | chr1: 1565891-1565987 |
| 220 | MN1 | chr22: 28197962-28198388 |
| 221 | MPZ | chr1: 161275561-161275996 |
| 222 | MSX2P1 | chr17: 56234436-56234516 |
| 223 | NACAD | chr7: 45128502-45128717 |
| 224 | NID2_A | chr14: 52535260-52535353 |
| 225 | NID2_B | chr14: 52535974-52536161 |
| 226 | NID2_C | chr14: 52536192-52536328 |
| 227 | NKX2-6 | chr8: 23564115-23564146 |
| 228 | NR2F6 | chr19: 17346428-17346459 |
| 229 | NTRK3 | chr15: 88800287-88800414 |
| 230 | NXPH4 | chr12: 57618904-57618944 |
| 231 | ODC1 | chr2: 10589075-10589243 |
| 232 | OLIG3_A | chr6: 137818896-137818917 |
| 233 | OLIG3_B | chr6: 137818978-137818988 |
| 234 | OSR2_A | chr8: 99952233-99952366 |
| 235 | OSR2_B | chr8: 99952801-99952919 |
| 236 | OSR2_C | chr8: 99960580-99960630 |
| 237 | OTX1.R | chr2: 63281481-63281599 |
| 238 | PAQR6 | chr1: 156215470-156215739 |
| 239 | PCDH8 | chr13: 53421299-53421322 |
| 240 | PDX1 | chr13: 28498503-28498544 |
| 241 | PDXK_A | chr21: 45148429-45148556 |
| 242 | PDXK_B | chr21: 45148575-45148681 |
| 243 | PEAR1 | chr1: 156863318-156863493 |
| 244 | PIF1 | chr15: 65116285-65116597 |
| 245 | PLXNC1_A | chr12: 94544327-94544503 |
| 246 | PLXNC1_B | chr12: 94544333-94544426 |
| 247 | POU4F1 | chr13: 79177505-79177532 |
| 248 | PPARA | chr22: 46545328-46545457 |
| 249 | PPARG | chr3: 12330042-12330152 |
| 250 | PPP1R16B_A | chr20: 37435507-37435716 |
| 251 | PPP1R16B_B | chr20: 37435738-37435836 |
| 252 | PPP2R5C | chr14: 102247681-102247929 |
| 253 | PRDM13_A | chr6: 100061616-100061742 |
| 254 | PRDM13_B | chr6: 100061748-100061792 |
| 255 | PRHOXNB | chr13: 28552424-28552562 |
| 256 | PRKCB | chr16: 23847575-23847699 |
| 257 | PRMT1 | chr19: 50179501-50179635 |
| 258 | PROM1 | chr4: 16084793-16085112 |
| 259 | PTPRM | chr18: 7568565-7568808 |
| 260 | PTPRN2 | chr7: 157483341-157483429 |
| 261 | RASGRF2 | chr5: 80256117-80256162 |
| 262 | RBFOX3_A | chr17: 77179579-77179752 |
| 263 | RBFOX3_B | chr17: 77179778-77180064 |
| 264 | RFX8 | chr2: 102090934-102091130 |
| 265 | RGS17 | chr6: 153452120-153452393 |
| 266 | RIC3.F | chr11: 8190622-8190711 |
| 267 | RIPPLY2 | chr6: 84563228-84563287 |
| 268 | RYR2_A | chr1: 237205369-237205428 |
| 269 | RYR2_B | chr1: 237205619-237205640 |
| 270 | SALL3 | chr18: 76739321-76739404 |
| 271 | SBNO2 | chr19: 1131795-1131992 |
| 272 | SCRT2_A | chr20: 644533-644618 |
| 273 | SCRT2_B | chr20: 644573-644618 |
| 274 | SERPINB9_A | chr6: 2902941-2902998 |
| 275 | SERPINB9_B | chr6: 2903031-2903143 |
| 276 | SLC16A3.F | chr17: 80189895-80189962 |
| 277 | SLC22A20.FR | chr11: 64993239-64993292 |
| 278 | SLC2A2 | chr3: 170746149-170746208 |
| 279 | SLC30A10 | chr1: 220101458-220101634 |
| 280 | SLC7A4 | chr22: 21386780-21386831 |
| 281 | SLC8A3 | chr14: 70654596-70654640 |
| 282 | SLITRK5.R | chr13: 88329960-88330076 |
| 283 | SNCA | chr4: 90758071-90758118 |
| 284 | SPHK2 | chr19: 49127580-49127683 |
| 285 | ST8SIA4 | chr5: 100240059-100240276 |
| 286 | STAC2_A | chr17: 37381217-37381303 |
| 287 | STAC2_B | chr17: 37381689-37381795 |
| 288 | STX16_A | chr20: 57224798-57224975 |
| 289 | STX16_B | chr20: 57225077-57225227 |
| 290 | SYN2 | chr3: 12045894-12045967 |
| 291 | SYNJ2 | chr6: 158402213-158402536 |
| 292 | SYT5 | chr19: 55690401-55690496 |
| 293 | TAL1 | chr1: 47697702-47697882 |
| 294 | TBKBP1 | chr17: 45772630-45772726 |
| 295 | TBX1 | chr22: 19754257-19754550 |
| 296 | TEPP | chr16: 58018790-58018831 |
| 297 | TIMP2 | chr17: 76921762-76921779 |
| 298 | TLX1NB | chr10: 102881178-102881198 |
| 299 | TMEFF2 | chr2: 193060012-193060126 |
| 300 | TMEM176A | chr7: 150497411-150497535 |
| 301 | TNFRSF10D | chr8: 23020896-23021114 |
| 302 | TOX | chr8: 60030723-60030754 |
| 303 | TRH_A | chr3: 129693484-129693575 |
| 304 | TRH_B | chr3: 129694457-129694501 |
| 305 | TRIM67 | chr1: 231297047-231297159 |
| 306 | TRIM71_A | chr3: 32858861-32858897 |
| 307 | TRIM71_B | chr3: 32859445-32859559 |
| 308 | TRIM71_C | chr3: 32860020-32860090 |
| 309 | TSHZ3 | chr19: 31839809-31840038 |
| 310 | UBTF | chr17: 42287924-42288018 |
| 311 | ULBP1 | chr6: 150285563-150285661 |
| 312 | USP44_A | chr12: 95942148-95942178 |
| 313 | USP44_B | chr12: 95942519-95942558 |
| 314 | UTF1 | chr10: 135044125-135044171 |
| 315 | UTS2R | chr17: 80329497-80329534 |
| 316 | VIPR2 | chr7: 158937370-158937481 |
| 317 | VN1R2 | chr19: 53758121-53758147 |
| 318 | VSNL1 | chr2: 17720216-17720257 |
| 319 | VSTM2B_A | chr19: 30016283-30016357 |
| 320 | VSTM2B_B | chr19: 30017789-30018165 |
| 321 | ZBTB16 | chr11: 113929882-113930166 |
| 322 | ZFP64 | chr20: 50721057-50721235 |
| 323 | ZNF132 | chr19: 58951402-58951775 |
| 324 | ZNF486 | chr19: 20278004-20278145 |
| 325 | ZNF626 | chr19: 20844070-20844199 |
| 326 | ZNF671 | chr19: 58238810-58238955 |
| 327 | ZSCAN12 | chr6: 28367128-28367509 |

Next, SYBR Green Methylation-specific PCR (qMSP) was performed on the discovery samples to confirm the accuracy and reproducibility of the candidate DMR's shown in Table 2.

qMSP primers were designed for each of the marker regions using Methprimer software (Li L C and Dahiya R. Bioinformatics. 2002 November; 18 (11): 1427-31) They were synthesized by IDT (Integrated DNA Technologies). Assays were tested and optimized (using the Roche LightCycler 480) on dilutions of bisulfite converted universally methylated DNA, along with converted unmethylated DNA and converted and unconverted leukocyte DNA negative controls (10 ng/ea). Assays taken forward needed to demonstrate linear regression curves and negative control values less than 5-fold below the lowest standard (1.6 genomic copies). Some of the more promising DMRs which had assay or control failures were re-designed. Of the 127 total designs (Table 3 shows the forward and reverse primer sequence information for the 127 total designs), 80 high performing MSP assays met QC criteria and were applied to the samples. The MSP primer sequences, each of which include 2-8 CpGs, were designed to provide a quick means of assessing methylation in the samples, and as such, were biased for amplification efficiency over trying to target the most discriminate CpGs-which would have required lengthy optimization timeframes.

DNA was purified as described in the discovery RRBS section and quantified using picogreen absorbance (Tecan/Invitrogen). 2 ug of sample DNA was then treated with sodium bisulfite and purified using the Zymo EZ-96 Methylation kit (Zymo Research). Eluted material was amplified on Roche 480 LightCyclers using 384-well blocks. Each plate was able to accommodate 2 markers (and standards and controls) for a total of 40 plates. The 80 MSP assays had differing optimal amplification profiles (Tm=60, 65, or 70° C.) and were grouped accordingly. The 20 uL reactions were run using LightCycler 480 SYBR I Master mix (Roche) and 0.5 umoles of primer for 50 cycles and analyzed, generally, by the Fit Point 18% absolute quantification method. All parameters (noise band, threshold, etc.) were pre-specified in an automated macro to avoid user subjectivity. The raw data, expressed in genomic copy number, was normalized to the amount of input DNA (β-actin). Results were analyzed logistically using JMP and displayed as AUC values. Twelve comparisons were run: each breast cancer subtype vs normal breast, and each subtype vs buffy coat. In addition, the methylation fold change ratio (mFCR) was calculated for each comparison using both average and median fractional methylation (FCR=cancer(methylated copies/β-actin copies)/normal(methylated copies/β-actin copies)). Both of these performance metrics were critical for assessing the potential of a marker in a clinical blood-based test.

>90% of the markers tested yielded superior performance in both AUC and FCR categories, with numerous AUCs in excess of 0.90, cancer vs normal tissue FCRs >10, and cancer vs buffy coat FCRs >50.

TABLE 3

| | DMR | Forward | SEQ ID | Reverse | SEQ ID |
|---|---|---|---|---|---|
| Gene Annotation | No. | Primer 5′-3′ | NO: | Primer 5′-3′ | NO: |
| AADAT-RS | 2 | GAG TTT CGG CGG | 1 | CGC TAC GTC TAA | 2 |
| | | CGT TTT TCG | | CTT CCC GCG C | |
| ABLIM1-FS | 3 | TTT TCG ACG AGT | 3 | GCG AAT CTA TCT | 4 |
| | | AGG ATT GAA GAA | | ACC GAA ACG CGC | |
| | | GGA AC | | T | |
| AJAP1_A | 6 | TTT TGA TTT GTA | 5 | GTA TAA ACG CGT | 6 |
| | | ATA TAG AGG AAA | | AAA TAC CAA ACT | |
| | | GCG TCG T | | AAA CGA A | |
| AJAP1_B | 7 | GTT TCG AGA AAG | 7 | ACT CCC AAC GAA | 8 |
| | | GAG AAG GGG GAG | | AAC TTC GCA AAC | |
| | | C | | G | |
| ALOX5-RS | 9 | GTT TTT TGT CGG | 9 | CCA AAA ATT AAA | 10 |
| | | GAG TTA TTC GT | | TTA AAA ACG CTA | |
| | | | | CGC A | |
| ASCL2-RS | 14 | GTT TTA GGA GGG | 11 | AAC ACG ACT ATT | 12 |
| | | TGG GGC GT | | CGA AAA ACG CGC | |
| | | | | A | |
| ATP6V1B1-RS | 15 | TTC GTA GTA TCG | 13 | GAA ATA ATA AAA | 14 |
| | | GGA GTC GA | | ACG CCG CAC GCT | |
| BANK1-FS | 17 | GTC GTA GTT TTC | 15 | CGA ACG CTA CCT | 16 |
| | | GCG GGT GGT AAG | | AAA CTC TCC CGA | |
| | | C | | C | |
| BEST4-RS | 20 | GGA ATC GCG AGT | 17 | AAA TAC AAT TAC | 18 |
| | | TTT GGG ATA GTC G | | ACC CTC TAC CGC | |
| | | | | C | |
| BHLHE23_C | 23 | GAG GCG TTC GGT | 19 | CCC CGA CCT ATA | 20 |
| | | GGG ATT TC | | AAC CTA CGA CGC | |
| | | | | T | |
| BHLHE23_D | 24 | GAG GAG GTA GCG | 21 | CGC GTC GAT CTA | 22 |
| | | GGC GTC GA | | ACT TAC CTA CGA | |
| | | | | A | |
| C10orf125-FS | 27 | TTG CGT TTA TCG | 23 | GCA CTA CTA TCC | 24 |
| | | ATT TCG TTT TCG T | | CCC GAA CTA CTC | |
| | | | | TAC GC | |
| C17orf64-RS | 29 | TTA TTA GGC GGG | 25 | CTC GAA TCC CTA | 26 |
| | | GAG TCG GGT GTC | | AAA AAC TCG CGA | |
| | | | | A | |
| C19orf66-FS | 31 | AGG AAA TTC GGT | 27 | AAA CCC CTA CAA | 28 |
| | | AGC GAT TAT ACG G | | CCT CAC CGT ACA | |
| | | | | CGA T | |
| CALN1_A | 36 | CGG AGT TAA TAG | 29 | CAA ACC CCC GAA | 30 |
| | | GTA CGG GAG GCG | | CTA TCG CGA A | |
| | | T | | | |
| CAPN2-FS | 39 | CGG GTA TCG CGG | 31 | TAT CGT AAA AAC | 32 |
| | | TTA AGT TGG C | | CCA ACC CCT CGA | |
| | | | | C | |
| CD1D-FS | 47 | GGG ATT GGT GAG | 33 | CTC CCC GAA ACC | 34 |
| | | ATT CGG GAC GT | | AAA AAA CAA CGA | |
| | | | | A | |
| CDH4_E | 53 | GTT TTA AAT CGT | 35 | ACG AAC GAA AAC | 36 |
| | | ATT CGT AGT TCG G | | TTT CCT AAA CGA | |
| | | | | A | |
| CHST2_A | 56 | GCG TTT TTT TAT | 37 | ACC GAC ACT ACC | 38 |
| | | CGT TTT AGG GCG T | | AAC CTC TCC GAA | |
| CHST2_B | 57 | TGC GGG GAT TTT | 39 | CCG ACG AAC TAT | 40 |
| | | TAG CGG AAG C | | CCG ACT ATC ACT | |
| | | | | CGT T | |
| CLIC6-FS | 58 | GTA GTA GGT GGA | 41 | CTC TCG AAA ACC | 42 |
| | | GGG GGC GAG TTC | | GCA AAA TCC TCG | |
| CLIP4-FS | 59 | GGT AAT ATT GCG | 43 | AAC AAT CAA ATA | 44 |
| | | ATA TTT CGT AGA | | ATC GAA CGC ACG | |
| | | CGT | | C | |
| COL23A1-RS | 60 | GTC GTT TTT CGT | 45 | AAA ACT AAA TAA | 46 |
| | | TAC GAA GCG GC | | ATC TAT CCT CGA | |
| | | | | T | |
| CXCL12-FS | 63 | GCG TCG GCG GTT | 47 | AAC GAA TCT CAT | 48 |
| | | TTT AGT AAA AGC | | TAA ATC TCC CGT | |
| | | | | C | |
| DBNDD1R-FS | 64 | GAT TTT CGG GAG | 49 | CTT CCC CGC AAC | 50 |
| | | CGG CGA | | GAA CCG | |
| DLX4-FS | 66 | TTC GTT GGT ATA | 51 | CGA ATA CCG AAA | 52 |
| | | TTC GCG TAG GTG | | TCT ATA ACC CCG | |
| | | C | | AA | |
| DLX6-FS | 67 | ATT ATG ATT ACG | 53 | CTC CAT AAA AAC | 54 |
| | | ATG GTT GAC GG | | GAA TTT AAA CGA | |
| | | | | A | |
| DNM3_A | 69 | TTT GGT TAT AGA | 55 | ATC GAA CCA CCA | 56 |
| | | ACG TAG AGG TCG | | AAC CAA ACG C | |
| | | T | | | |
| DSCR6-FS | 72 | GGG AAG TTT AGT | 57 | ACT AAA AAC GTT | 58 |
| | | AGG TGA GCG T | | TCC GTC GAA CGC | |
| | | | | A | |
| DTX1-RS | 73 | GTT GGT AGG AGT | 59 | ATC GCA ATC GTA | 60 |
| | | AGG GTT GGT TCG | | ACC CGT AAA CGC | |
| | | A | | | |
| EMX1_A | 74 | ATT CGT ACG GTT | 61 | GAC CAA CTA CTT | 62 |
| | | TTT TCG TTT TCG T | | CCG CTC GAC GC | |
| ETS1_B | 80 | CGG ATT TAG CGG | 63 | TTT AAA ACG TTT | 64 |
| | | TCG AGA CG | | CTC GCG ACG CC | |
| FAM126A-FS | 83 | TCG TTA GGC GAT | 65 | TAA AAA AAC CAT | 66 |
| | | GAT AAT TAG CGA | | AAA CCC TAA CGA | |
| | | | | C | |
| FAM129C-FS | 84 | GTT GGA GAA GAC | 67 | CCA AAA CCT CAC | 68 |
| | | GAT TCG TTC GGA C | | TCC TCA ACC GC | |
| FBN1-FS | 91 | CGC GAT GCG CGT | 69 | GAC GCG ACT AAC | 70 |
| | | TTT GAA C | | TTC CAA CCT AAC | |
| | | | | GAA | |
| FMN2-RS | 94 | TTT TCG TGG TTG | 71 | GCC GCG CTC TAC | 72 |
| | | TCG TCG TTG C | | ACT AAA CAT ATT | |
| | | | | CGC | |
| FOXP4-FS | 96 | CGG GGA AGT GGG | 73 | AAA AAA ACT AAA | 74 |
| | | AGT TTT TAG CG | | TCA AAA CCG CGA | |
| | | | | C | |
| GAS7-FS | 99 | GCG AGT TCG CGT | 75 | ACC GAC GCT ACC | 76 |
| | | TGT TTA CGT TTC | | TAT AAC TCC ACG | |
| | | | | CT | |
| GP5-RS | 104 | TTA GGT TTG TTT | 77 | TCT ACA AAA CGC | 78 |
| | | ATT AAT TTT ACG T | | CGC GAC | |
| GRM7-FS | 106 | GTT AAT TCG AGA | 79 | GAC CAA AAA AAA | 80 |
| | | GCG CGA GGC GT | | TAA AAA ATC CCG | |
| | | | | CGA C | |
| GYPC_B | 109 | TAA AGA AAT AGA | 81 | CGA ACT AAA AAA | 82 |
| | | AAG CGG GCG ATA | | ACC GCC AAC CCG | |
| | | CGT | | | |
| HHEX-RS | 113 | GGG TTT TGC GGT | 83 | AAT AAC AAA CGC | 84 |
| | | TAA TGG CG | | GTC CCG AAA ACG | |
| | | | | A | |
| HNF1B_B | 116 | TTA GTT TTT TTT | 85 | AAC TTT TCC ACC | 86 |
| | | GGT TTT TAT TTG | | GAT TCT CAA TTC | |
| | | AAT TTC GA | | CG | |
| HOXA1_A | 117 | ATT TAA ATT TTC | 87 | ACA CTC CAA ATC | 88 |
| | | GGC GTT TCG TCG | | GAC CTT TAC AAT | |
| | | T | | CGC | |
| HOXA7_A | 119 | AGT TTG GTT CGT | 89 | AAC GCG ACT AAA | 90 |
| | | TTA GCG ATT GCG T | | ACC AAT TTC CGC | |
| | | | | A | |
| IGF2BP3_A | 123 | TTT ATT TGT TTT | 91 | AAA TAT ATA CCC | 92 |
| | | TAT CGT TCG TCG G | | GAT TTC CCC GTT | |
| IGF2BP3_B | 124 | TAA TCG GCG TCG | 93 | CCG TCA ACC AAT | 94 |
| | | AGA GAG ATA TCG T | | CGA AAA CGA A | |
| IL15RA-FS | 128 | TCG TTT ATT TCG | 95 | AAC CAA CCT AAA | 96 |
| | | TTT TTT TTG TCG A | | ATC TAC ACT CGC | |
| | | | | A | |
| ITPRIPL1-FS | 134 | GGG TCG TAG GGG | 97 | CAT ACT TAT CCG | 98 |
| | | TTT ATC GC | | AAC GTC TAA ACG | |
| | | | | TC | |
| ITPRIPL1-FS | 134 | GGT TTT AGC GAT | 99 | CAC GAT CTT AAA | 100 |
| | | GAA TCG GAC GT | | AAA ACA ACG CGA | |
| | | | | C | |
| KCNH8-RS | 138 | CGT ATT TTT AGG | 101 | ACA CTA TTA CCC | 102 |
| | | TTT AGT TCG GCG T | | GCG AAA AAA CGA | |
| | | | | T | |
| KCNK17_B | 140 | GAG TTT GTT TGG | 103 | CCA AAT ATA ACG | 104 |
| | | GGG TTG GTC GTA | | TTT AAC TCT TTA | |
| | | TTC | | CCA CGA A | |
| KCNK9-FS | 141 | TTT TTT TTG ATT | 105 | CTA ATA AAC GCC | 106 |
| | | CGG ATT TTT TCG G | | GCC GTA TTC GAC | |
| | | | | G | |
| KLF16-FS | 145 | TTT TCG CGT TGT | 107 | TAC ACA ACC ACC | 108 |
| | | TTT TAT TTA TCG T | | CAA CTA CTC CGC | |
| | | | | G | |
| KLHDC7B-RS | 146 | TGT TGT TGG GTA | 109 | CGA AAA CCC AAC | 110 |
| | | AAG GTT AGT ACG T | | TCC CGA A | |
| LAYN-RS | 147 | TTT TTG CGG TCG | 111 | CTT ACC AAC TAA | 112 |
| | | TTT TTC GGA GC | | CCC CCG CCT ACC | |
| | | | | G | |
| LIME1-RS | 148 | CGT TTT AGT AGG | 113 | CCC GAA AAC CAA | 114 |
| | | GAT TGG GGG CGA | | AAT AAA ATC CGC | |
| | | | | A | |
| LMX1B_A | 149 | CGG AAT AGC GCG | 115 | TTT AAC CGT AAC | 116 |
| | | GTC GTT TTT TC | | GCT CGC CTC GAC | |
| LOC100132891-FS | 153 | GTC GGT TGT GTT | 117 | AAA AAA AAC CCC | 118 |
| | | TAG AGC GTA GCG | | GAC GAC GAA | |
| | | T | | | |
| LOC100132891-FS | 153 | GTT GCG ATT GTT | 119 | ATA ATA ACA AAA | 120 |
| | | TGT ATT TTG CGG | | AAC CCC TCC CGA | |
| | | | | C | |
| LSS-FS | 157 | AGT TTC GTT AGG | 121 | CAA CTA AAA CTC | 122 |
| | | GAA GGG TTG CGT | | TAC CGC GCT CGA | |
| | | C | | T | |
| MAGI2-RS | 159 | AGG AAG GGT TTC | 123 | AAA AAA ATC AAC | 124 |
| | | GAG TTT AGT GCG | | GCG TCC TCC TCG | |
| | | G | | C | |
| MAST1-RS | 160 | TTT CGA TTT CGT | 125 | AAA CTA AAC GAC | 126 |
| | | TTT TAA ATT TCG T | | CTA ACC CTA CGT | |
| | | | | A | |
| MAX.chr1.8277479- | 166 | AAG TTT ACG CGC | 127 | CGA AAC GAC TTC | 128 |
| 8277527-RS | | GAG TTT GAT CGT C | | TCT CCC CGC A | |
| MAX.chr11.14926602- | 168 | TTT AGT TCG CGG | 129 | GAA AAC ACA ATA | 130 |
| 14927148-FS | | AAG TTA GGT TCG G | | AAC CCC GCC GTC | |
| MAX.chr11.68622869- | 169 | GTT AGA TTG TAG | 131 | AAA AAA CGA CTA | 132 |
| 68622968-FS | | GAG GGA TTA GCG | | AAA AAT TCA CGC | |
| | | G | | C | |
| MAX.chr12.4273906- | 170 | TTT GGA GTT TGG | 133 | CGA CGA AAC TAA | 134 |
| 4274012-FS | | GGG ATC GAT AGT | | AAC CGC GTA CGT | |
| | | C | | A | |
| MAX.chr12.4273906- | 170 | TTT GGA GTT TGG | 135 | CGA CGA AAC TAA | 136 |
| 4274012-FS | | GGG ATC GAT AGT | | AAC CGC GTA CGT | |
| | | C | | A | |
| MAX.chr12.59990671- | 171 | ATT ATA TTG GGG | 137 | AAC AAA CAA TTC | 138 |
| 59990859-FS | | GCG TTA GGT TCG | | GCA CGT AAA CGA | |
| | | G | | A | |
| MAX.chr15.96889013- | 173 | GGG CGG TTT ACG | 139 | GCG TCT CGA ACC | 140 |
| 96889128-FS | | TGG ATT TTT ATA | | GTA CCC TAA CGT | |
| | | GAT TTT C | | A | |
| MAX.chr17.73073682- | 174 | CGT CGT TGT TGA | 141 | CGC TTC CTA ACA | 142 |
| 73073814-RS | | TTA TGA TCG CGG | | ACC TTC CTC GAA | |
| MAX.chr18.76734362- | 177 | TTA ACG GTA TTT | 143 | AAA AAA AAC TCG | 144 |
| 76734476-RS | | TTT GTT TTT TCG T | | TCC CCG CGC T | |
| MAX.chr19.46379903- | 179 | TCG GTT AGT TCG | 145 | TAT TAA CCG AAA | 146 |
| 46380197-FS | | AGG TAG GAA GTT | | AAC GAA AAC CAA | |
| | | TTG C | | ATC CGA | |
| MAX.chr19.46379903- | 179 | AGT TTT GTT GTT | 147 | AAA AAC TAA AAA | 148 |
| 46380197-FS | | TTG GGT AGG TCG | | CCT TTC TCT CGA | |
| | | G | | C | |
| MAX.chr2.223183057- | 180 | GCG TTG AGA GTG | 149 | ACT ACC TAA ACT | 150 |
| 223183114-RS | | ACG GAT ATT TTT | | CCG AAC ACG CCC | |
| | | CGT C | | G | |
| MAX.chr20.1784209- | 185 | TTA GCG TAT CGG | 151 | GAA AAC GAA AAA | 152 |
| 1784461-FS | | GAA TTA GGG GGA | | ACG ACG CGC A | |
| | | C | | | |
| MAX.chr20.1784209- | 185 | TCG TTT TTT AGG | 153 | GAA CCG TAT TTA | 154 |
| 1784461-RS | | TGG GGA AGA AGC | | AAA CCA ATC CCC | |
| | | G | | GC | |
| MAX.chr4.8859602- | 191 | AAT TGG GGT TCG | 155 | TTA CCC CTA CCC | 156 |
| 8859669-RS | | GGG TTC GGT AC | | AAA AAA ATA CGC | |
| | | | | T | |
| MAX.chr5.145725410- | 193 | GGG GTT AGA GTT | 157 | CGC GTC TCC CGT | 158 |
| 145725459-RS | | TCG CGT TCG C | | CCT ATC TAT ATA | |
| | | | | CGT C | |
| MAX.chr5.42994866- | 198 | TAG GAA TTT TTT | 159 | CAC AAA AAC TCG | 160 |
| 42994936-FS | | AAA TTC GTT TTA | | ATA CAA TTA CCG | |
| | | CGG | | TT | |
| MAX.chr5.77268672- | 199 | TAT TTT ATA GTC | 161 | GTC GAT AAA AAA | 162 |
| 77268725-FS | | GCG TTA AAA GCG T | | CCT ACG CGA CGA | |
| | | | | A | |
| MAX.chr6.157557371- | 204 | GAT TTA GTT TTT | 163 | TAT TAA AAA CGA | 164 |
| 157557657-FS | | CGG GTT TAT AGC | | CCA AAC CTC CGC | |
| | | GG | | A | |
| MAX.chr8.124173030- | 208 | TGG TTG TAG GCG | 165 | AAA AAC GAC CCT | 166 |
| 124173395-FS | | TTT TGT TGG AGT | | AAC CAC CCT CGT | |
| | | TC | | T | |
| MCF2L2-FS | 216 | TTT TGC GTA GTT | 167 | CCC GCA TTC CCG | 168 |
| | | GGG TAG GGT TCG | | AAA AAA ACG AT | |
| | | G | | | |
| MCF2L2-RS | 216 | TTA GGG TTT TTT | 169 | ATC CCC CGT ACG | 170 |
| | | TCG AGG AGT TCG | | AAA CTA AAC GCG | |
| | | A | | | |
| MCF2L2-RS | 216 | GCG TTC GTA TTT | 171 | TCT ACG TAA CTA | 172 |
| | | TCG GGA GAG GC | | AAC AAA ACC CGA | |
| | | | | A | |
| MIB2-FS | 219 | CGT TTT GTG TTT | 173 | AAA ACC CCA AAA | 174 |
| | | TAT AAA AAG AAA | | ACG CCC GAT | |
| | | GAT TTT CGG | | | |
| MPZ-FS | 221 | GGG GCG TAT ATA | 175 | AAA AAA AAC CCT | 176 |
| | | TTA GTT ATC GAG | | AAA AAC CGC CGA | |
| | | CGA | | A | |
| MSX2P1-FS | 222 | TTC GTT TAA TGA | 177 | TAA AAC AAA CTA | 178 |
| | | GAA GGG GTT AGC | | AAA ACC TTA ACG | |
| | | GG | | CGA CGC T | |
| NACAD-RS | 223 | GGG GAG GGA GTT | 179 | GTA CGC GAA CTC | 180 |
| | | TTT TTT AC | | GCC AAA CAC TAC | |
| | | | | G | |
| ODC1-FS | 231 | GTA GGG TTG GTA | 181 | AAC CCA TCT AAT | 182 |
| | | GTC GTT TTT ACG T | | TAC AAA ATA CCT | |
| | | | | CGA T | |
| ODC1-RS | 231 | GGT TTT ATA GGG | 183 | AAA ACC TCG TCT | 184 |
| | | GAA ATT ATT TTC | | TTA TAA CAT CGA | |
| | | GT | | A | |
| ODC1-RS | 231 | TAG GAT ATT TCG | 185 | AAC AAA ACT AAC | 186 |
| | | ATG TTA TAA AGA | | AAC CGC CTC CAC | |
| | | CGA | | G | |
| OSR2_A | 234 | TTT GGA GTT ATC | 187 | GCA CGC CGA AAA | 188 |
| | | GGA AGG CGA AAG | | AAT AAA AAC GAA | |
| | | TAC | | | |
| OTX1-RS | 237 | TTT TCG ATA TCG | 189 | ATA ACT TAA AAC | 190 |
| | | ATA TCG AAG GCG T | | CCT AAA TTC CGC | |
| | | | | C | |
| PAQR6-FS | 238 | GCG GGT AGT AGG | 191 | CCG ACT TCC GTA | 192 |
| | | AAG ATT AGT AGC | | CGA AAC CGT A | |
| | | GG | | | |
| PLXNC1_A | 245 | TAA TAG AGG TTT | 193 | AAC GCA CCC TAA | 194 |
| | | GCG TTG GAA TCG | | ACA AAA CCA CGA | |
| | | A | | C | |
| PLXNC1_B | 246 | TGA AGA GTT GTT | 195 | GCC AAA AAT TCG | 196 |
| | | AGT TCG TTT AGC | | ATT CCA ACG CA | |
| | | GT | | | |
| PPARA-FS | 248 | TAG TGG TAG GTA | 197 | ATC AAA ACT CCC | 198 |
| | | TAG TTG GTA GCG | | CTC CTC GAA AAC | |
| | | G | | G | |
| PPARG-RS | 249 | GTT TTT AAG CGG | 199 | AAA AAA AAT CCC | 200 |
| | | CGG TCG T | | GTT CGC T | |
| PRKCB-RS | 256 | GCG CGC GTT TAT | 201 | AAA ATC AAA AAC | 202 |
| | | TAG ATG AAG TCG | | CAC AAA TTC ACC | |
| | | | | GCC | |
| PRMT1-FS | 257 | CGG GGA GAG GAG | 203 | CAA CTT AAA CAC | 204 |
| | | GGG TAG GAT TTA C | | CAC TTC CTC CGA | |
| | | | | A | |
| RBFOX3_A | 262 | TGT TTT TTT TGT | 205 | AAA TAA CTA ACT | 206 |
| | | TCG GGC GG | | CCT ACT CTC GCC | |
| | | | | CGC T | |
| RFX8-FS | 264 | ATA GTT TTT TAA | 207 | AAA AAC AAC TCC | 208 |
| | | TTT TCG CGT TTC | | AAC CCA CAC CGC | |
| | | GTC GA | | | |
| RIC3-RS | 266 | GCG GGA GGA GTA | 209 | AAA AAC AAA ATA | 210 |
| | | GGT TAA TTT TCG A | | CGC GAA ACG CAC | |
| | | | | G | |
| SCRT2_B | 273 | CGA GAA GGT TTT | 211 | TAC GTA TCC ATA | 212 |
| | | GTC GTA GAC GTC | | CCC GCG CTC G | |
| | | GT | | | |
| SLC16A3-FS | 276 | TTT GTT TGT ATA | 213 | CGC CTA ACT ACC | 214 |
| | | ATA GGG GTT GCG | | GAA AAA TAC CGA | |
| | | G | | A | |
| SLC22A20-FS | 277 | GGT GGG GTT ATT | 215 | CGA ACC AAA CCT | 216 |
| | | TTT TTA TGG AGT | | ACG ATT CCC GAA | |
| | | CGA TTC | | | |
| SLC2A2-RS | 278 | GGG AGA AGA GAA | 217 | TCT TAT ACT CAA | 218 |
| | | TGG TTT TTT GTC | | CCC CGA CCT ACC | |
| | | GTC | | GAC | |
| SLC30A10-FS | 279 | GTT TTA TTC GGG | 219 | AAA AAA CCG CGT | 220 |
| | | GTT TTA GCG TTA | | TAC TCA ACG CGC | |
| | | TTT ACG G | | | |
| SLC7A4-RS | 280 | GTT TAG AGC GGA | 221 | CGC CTA TTC TTA | 222 |
| | | GGT AGC GGT TGC | | AAC CTA AAC CCG | |
| | | | | TC | |
| SLITRK5-FS | 282 | CGT AGA GGA TTA | 223 | TAC TAT AAC TAC | 224 |
| | | TAA AGA TTT GTA | | TAC GAT AAC GAC | |
| | | CGA | | GAC GAC | |
| SPHK2-RS | 284 | AGA TTT CGG TTT | 225 | ATT AAT ACT AAC | 226 |
| | | TTG TTT CGA TTT | | TTA CGA AAC CGC | |
| | | TCG T | | C | |
| ST8SIA4-RS | 285 | ATT ATT TTT GAG | 227 | AAA TTT CTC TCC | 228 |
| | | CGT GAA AAA TCG T | | AAT TAA ATT CCG | |
| | | | | TA | |
| STAC2_B | 287 | GTG GGT TTG TCG | 229 | AAA TAA CCG CGT | 230 |
| | | TCG GAT TTC G | | CAT CCG ATT CGT | |
| | | | | T | |
| STX16_A | 288 | TGG ATG TTT TAT | 231 | GTA CTT TTT CTC | 232 |
| | | ATT AAT TTT TAG | | TCA CGA AAA ATA | |
| | | TTG TAT AAC G | | TTC CCG C | |
| STX16_B | 289 | TGC GTG GAA TAA | 233 | GCT CAA CAC ACG | 234 |
| | | ATT TTA TAT ACG T | | AAA AAC CCT CGA | |
| | | | | A | |
| STX16_B | 289 | CGG TGC GGG GTT | 235 | TCC ACG CAA AAA | 236 |
| | | TTA ATA AAG GAT C | | CAA AAA ACG CGT | |
| | | | | A | |
| SYNJ2-FS | 291 | GGC GTA GTT ATG | 237 | ATC CTT TCG ACC | 238 |
| | | ATT TCG TTT TTT | | CTA CGT ACC TCG | |
| | | CGT | | AT | |
| TBX1-FS | 295 | TTT ACG ATT ATT | 239 | GAA CCC GAC GAA | 240 |
| | | GTT TTA GAT AAT | | CTT CGA A | |
| | | ACG G | | | |
| TMEM176A-FS | 300 | GGG AAA TCG CGT | 241 | AAA ACG ACG AAA | 242 |
| | | AGT TTG GGC | | AAA CGA AAA CGA | |
| | | | | C | |
| TNFRSF10D-FS | 301 | AGT TAT CGC GAT | 243 | AAA CGA TTA CCT | 244 |
| | | CGG TTT GGG TTA | | CTT TCG TTC GTT | |
| | | AC | | CGT T | |
| TRH_A | 303 | CGG CGG TTT ATT | 245 | CGA CAA ATC AAA | 246 |
| | | TGA AGA GGG TTC | | AAT CTA CAA CGC | |
| | | | | T | |
| TRIM67-RS | 305 | TTT TAA CGT TAG | 247 | CGA ACA AAC CAA | 248 |
| | | TTA CGA GTT GCG | | ACA ACC GAA | |
| | | G | | | |
| UBTF-RS | 310 | GTA GAT TAG GCG | 249 | GAA CAA AAA CAT | 250 |
| | | GGG GCG A | | AAA CTA ATA CAA | |
| | | | | ATA TCT CCC G | |
| ZSCAN12-FS | 327 | GGA GGG AGA GTT | 251 | CTA AAC CCC TCA | 252 |
| | | TTT CGC GGA TTC | | AAC CCT AAC CGA | |
| | | | | T | |
| GRASP | 105 | TGT TTT CGG ATA | 253 | ACG AAC GAA CTA | 254 |
| | | CGG CGA GC | | TAC GCG ACG CT | |

### Example II

This example describes the tissue validation of breast-cancer specific markers. Independent tissue samples (fresh frozen) were selected from institutional cancer registries at Mayo Clinic Rochester and were reviewed by an expert pathologist to confirm correct classification and to guide macro-dissection.

55 methylated DNA markers (MDMs) were chosen from the list of 80 MDMs (see, Example I) which were tested on the discovery samples.

Genomic DNA was prepared using QIAamp DNA Mini Kits (Qiagen, Valencia CA) and bisulfite converted using the EZ-96 DNA Methylation kit (Zymo Research, Irvine CA). Amplification primers were designed from marker sequences using Methprimer software (University of California, San Francisco CA) and synthesized commercially (IDT, Coralville IA). Assays were rigorously tested and optimized by SYBR Green qPCR (Roche) on bisulfite converted (methylated and unmethylated genomic DNA) and unconverted controls. Assays which cross reacted with negative controls were either redesigned or discarded. Melting curve analysis was utilized to ensure specific amplification was occurring.

qMSP was performed using the LightCycler 480 instrument on 2 μL of converted DNA in a total reaction volume of 25 uL. Standards were derived from serially diluted universal methylated DNA (Zymo Research). Raw marker copies were standardized to CpG-agnostic β-actin, a marker for total genomic DNA.

Results were analyzed logistically using JMP10 (SAS, Cary NC). Cases were compared separately to normal breast controls and normal buffy coat samples. Methylation ratios and absolute differentials were calculated for each of the MDMs.

MDM performance in the independent samples was excellent with many AUCs and methylation fold change ratios (FCs) greater than 0.90 and 50, respectively. Results are provided in Table 4 (Overall). Here, the MDMs are ranked by AUC (comparing overall cases to buffy coat samples). This is a critical metric for potential application in plasma as the majority of cell-free DNA (cfDNA) originates with leukocytes. Any MDM which does not highly discriminate epithelial-derived cancers from leukocyte DNA will fail in a blood test format, no matter its performance in tissues. 41 of 55 MDMs had cancer v buffy coat AUCs in excess of 0.9, with 3 achieving perfect discrimination (AUC=1). Table 4 also list AUCs, FCs, p-values, and % cancer methylation as other critical metrics in evaluating and demonstrating the excellence of these MDMs.

TABLE 4

| | Overall | | % | | DMR |
|---|---|---|---|---|---|
| Gene Annotation | AUC | p-value | meth | FC | No. |
| ATP6V1B1 | 0.88731 | <.0001 | 26.75 | 3.17 | 15 |
| FOXP4 | 0.62969 | 0.0032 | 47.95 | 1.39 | 96 |
| LMX1B_A | 0.86181 | <.0001 | 26.52 | 3.35 | 149 |
| BANK1 | 0.81125 | <.0001 | 28.59 | 2.25 | 17 |
| OTX1 | 0.84786 | <.0001 | 28.23 | 3.84 | 237 |
| ST8SIA4 | 0.61072 | 0.0054 | 19.51 | 1.59 | 285 |
| MAX.chr11.14926602-14927148 | 0.93745 | <.0001 | 18.72 | 33.52 | 168 |
| UBTF | 0.81517 | <.0001 | 42.18 | 3.07 | 310 |
| STX16_B | 0.66565 | <.0001 | 38.93 | 2.61 | 289 |
| KLHDC7B | 0.67241 | 0.0005 | 29.94 | 1.58 | 146 |
| PRKCB | 0.92153 | <.0001 | 19.52 | 43.21 | 256 |
| TBX1 | 0.36127 | 0.9266 | 13.81 | 1.02 | 295 |
| TRH_A | 0.94355 | <.0001 | 29.05 | 11.02 | 303 |
| MPZ | 0.93396 | <.0001 | 18.93 | 65.72 | 221 |
| GP5 | 0.79294 | <.0001 | 30.09 | 4.08 | 104 |
| DNM3_A | 0.85418 | <.0001 | 24.75 | 30.48 | 69 |
| MAX.chr17.73073682-73073814 | 0.53095 | 0.1372 | 21.71 | 1.31 | 174 |
| TRIM67 | 0.91391 | <.0001 | 10.80 | 41.41 | 305 |
| PLXNC1_A | 0.76983 | <.0001 | 10.49 | 16.29 | 245 |
| MAX.chr12.4273906-4274012 | 0.9017 | <.0001 | 12.09 | 55.76 | 170 |
| CALN1_A | 0.87271 | <.0001 | 11.47 | 27.59 | 36 |
| ITPRIPL1 | 0.88928 | <.0001 | 17.19 | 37.21 | 134 |
| MAX.chr12.4273906-4274012 | 0.9029 | <.0001 | 6.69 | 197.30 | 170 |
| GYPC_B | 0.87925 | <.0001 | 15.78 | 16.22 | 109 |
| MAX.chr5.42994866-42994936 | 0.8932 | <.0001 | 11.11 | 16.19 | 198 |
| OSR2_A | 0.80667 | <.0001 | 18.56 | 44.40 | 234 |
| SCRT2 | 0.841 | <.0001 | 7.84 | 59.82 | 273 |
| MAX.chr5.145725410-145725459 | 0.91303 | <.0001 | 10.12 | 52.12 | 193 |
| MAX.chr11.68622869-68622968 | 0.87947 | <.0001 | 20.07 | 23.21 | 169 |
| MAX.chr8.124173030-124173395 | 0.85636 | <.0001 | 21.94 | 3.08 | 208 |
| CXCL12 | 0.60615 | <.0001 | 41.39 | 6.66 | 63 |
| MAX.chr20.1784209-1784461 | 0.85113 | <.0001 | 12.03 | 36.91 | 185 |
| LOC100132891 | 0.89124 | <.0001 | 19.91 | 64.60 | 153 |
| BHLHE23_D | 0.82149 | <.0001 | 5.60 | 86.71 | 24 |
| ALOX5 | 0.79948 | <.0001 | 15.96 | 14.97 | 9 |
| MAX.chr19.46379903-46380197 | 0.84416 | <.0001 | 12.85 | 31.77 | 179 |
| ODC1 | 0.76024 | <.0001 | 7.77 | 16.38 | 231 |
| CHST2_B | 0.84154 | <.0001 | 12.15 | 226.06 | 57 |
| MAX.chr5.77268672-77268725 | 0.90519 | <.0001 | 12.13 | 46.85 | 199 |
| C17orf64 | 0.87293 | <.0001 | 28.03 | 31.07 | 29 |
| EMX1_A | 0.88056 | <.0001 | 11.01 | 83.60 | 74 |
| CHST2_A | 0.77114 | <.0001 | 8.00 | 97.42 | 56 |
| DSCR6 | 0.86595 | <.0001 | 7.14 | 73.34 | 72 |
| ITPRIPL1 | 0.88165 | <.0001 | 15.26 | 36.79 | 134 |
| IGF2BP3_B | 0.81822 | <.0001 | 27.51 | 69.74 | 124 |
| CDH4_E | 0.78073 | <.0001 | 6.67 | 12.81 | 53 |
| NACAD | 0.75207 | <.0001 | 4.29 | 38.67 | 223 |
| DLX4 | 0.86399 | <.0001 | 22.31 | 9.58 | 66 |
| ABLIM1 | 0.83054 | <.0001 | 5.25 | 265.54 | 3 |
| BHLHE23_C | 0.79174 | <.0001 | 7.40 | 66.61 | 23 |
| MAST1 | 0.73627 | <.0001 | 9.73 | 32.31 | 160 |
| ZSCAN12 | 0.75774 | <.0001 | 7.79 | 139.40 | 327 |
| SLC30A10 | 0.78182 | <.0001 | 8.75 | 55.48 | 279 |
| GRASP | 0.77114 | <.0001 | 7.10 | 43.44 | 105 |
| C10orf125 | 0.72646 | <.0001 | 11.36 | 9.94 | 27 |

### Example III

This example describes identification of breast tissue markers and plasma markers for detecting breast cancer.

Candidate methylation markers for the detection of breast cancer were identified by RRBS of breast cancer and normal breast tissue samples. Originally 58 markers were identified and target enrichment long-probe quantitative amplified signal assays were designed and ordered (see, e.g., WO2017/075061 and U.S. patent application Ser. No. 15,841,006 for general techniques) (Table 5 shows the methylated regions distinguishing breast cancer tissue from normal breast tissue) (Tables 6 and 7 show the primer and probe sequences for the markers shown in Table 5). After design screening and redesign, 56 markers (see, Table 8) were chosen and assays made, triplexed and tested on tissue. Assays were equally split between FAM and HEX reporting and triplexed with the reference assay, B3GALT6 which reports to Quasar670.

TABLE 5
Methylated regions distinguishing breast cancer tissue
from normal breast tissue

| DMR | | Region on Chromosome |
|---|---|---|
| No. | Gene Annotation | (starting base-ending base) |
| 329 | ABLIM1_B | chr10: 116391634-116391781 |
| 330 | AJAP1_C | chr1: 4715533-4715652 |
| 331 | ALOX5_B | chr10: 45914740-45914889 |
| 332 | ASCL2_B | chr11: 2292232-2292371 |
| 333 | BANK1_B | chr4: 102711861-102712082 |
| 334 | BHLHE23_E | chr20: 61638334-61638574 |
| 335 | C10orf125_B | chr10: 135171404-135171514 |
| 336 | C17orf64_B | chr17: 58499085-58499196 |
| 337 | CALN1_1520 | chr7: 71801485-71801604 |
| 37 | CALN1_B | chr7: 71801741-71801800 |
| 339 | CD1D_1058 | chr1: 158150861-158151139 |
| 340 | CDH4_7890 | chr20: 59827763-59828158 |
| 341 | CHST2_8128 | chr3: 142838015-142838501 |
| 342 | CHST2_8384 | chr3: 142838015-142838501 |
| 343 | CHST2_9316 | chr3: 142839218-142839575 |
| 344 | CHST2_9470 | chr3: 142839218-142839575 |
| 345 | CLIC6_B | chr21: 36042020-36042140 |
| 346 | CXCL12_B | chr10: 44881200-44881315 |
| 347 | DLX4_B | chr17: 48042552-48042616 |
| 348 | DNM3_D | chr1: 171810425-171810575 |
| 74 | EMX1_A | chr2: 73151498-73151578 |
| 349 | ESPN_B | chr1: 6507924-6508087 |
| 350 | FAM59B_7764 | chr2: 26407703-26407976 |
| 351 | FOXP4_B | chr6: 41528816-41528912 |
| 104 | GP5 | chr3: 194118738-194118924 |
| 352 | HOXA1_C | chr7: 27135593-27135895 |
| 353 | IGF2BP3_C | chr7: 23513861-23514064 |
| 354 | IPTRIPL1_1138 | chr2: 96990958-96991338 |
| 355 | IPTRIPL1_1200 | chr2: 96990958-96991338 |
| 356 | KCNK9_B | chr8: 140715096-140715177 |
| 357 | KCNK17_C | chr6: 39281887-39281994 |
| 358 | KLHDC7B_B | chr22: 50987209-50987311 |
| 359 | LAYN_B | chr11: 111412023-111412090 |
| 360 | LIME1_B | chr20: 62369173-62369342 |
| 361 | LMX1B_D | chr9: 129388170-129388223 |
| 362 | LOC100132891_B | chr8: 72755986-72756299 |
| 375 | MAST1_B | chr19: 12978496-12978642 |
| 338 | MAX.chr12.427.br | chr12: 4273906-4274012 |
| 174 | MAX.chr17.73073682-73073814 | chr17: 73073682-73073814 |
| 363 | MAX.chr20.4422 | chr20: 1784207-1784471 |
| 364 | MPZ_5742 | chr1: 161275554-161276006 |
| 365 | MPZ_5554 | chr1: 161275554-161276006 |
| 366 | MSX2P1_B | chr17: 56234426-56234520 |
| 367 | ODC1_B | chr2: 10589075-10589225 |
| 234 | OSR2_A | chr8: 99952233-99952366 |
| 368 | OTX1_B | chr2: 63281460-63281599 |
| 246 | PLXNC1_B | chr12: 94544333-94544426 |
| 369 | PRKCB_7570 | chr16: 23847569-23847705 |
| 370 | SCRT2_C | chr20: 644563-644631 |
| 279 | SLC30A10 | chr1: 220101458-220101634 |
| 371 | SPHK2_B | chr19: 49127571-49127685 |
| 372 | ST8SIA4_B | chr5: 100240049-100240286 |
| 373 | STX16_C | chr20: 57225077-57225237 |
| 374 | TBX1_B | chr22: 19754226-19754419 |
| 303 | TRH_A | chr3: 129693484-129693575 |
| 328 | TRIM67_B | chr1: 231297039-231297163 |

TABLE 6

| | | | SEQ | | SEQ |
|---|---|---|---|---|---|
| Gene | DMR | Forward | ID | Reverse | ID |
| Annotation | No. | Primer 5′-3′ | NO: | Primer 5′-3′ | NO: |
| ABLIM1_B | 329 | TGGTAATCGGGTTTTT | 255 | CCGCGAATCTATCTACC | 256 |
| | | CGACG | | GAAAC | |
| AJAP1_C | 330 | GTGTTAGGTTGGGCGG | 257 | GTTACCCGCTTACGAAA | 258 |
| | | AAG | | AACGA | |
| ALOX5_B | 331 | TTCGTTTTTTGTCGGG | 259 | TCCAAAAATTAAATTAAA | 260 |
| | | AGTTATTC | | AACGCTACGC | |
| ASCL2_B | 332 | ATAATACGGTTGTTCG | 261 | GTAAATATAAACTACGCG | 262 |
| | | GGAGG | | ACGCGTA | |
| BANK1_B | 333 | GAGAGTTTAGGTAGCG | 263 | CCTAACGCTACTAACAAC | 264 |
| | | TTCGG | | ATTATAACGA | |
| BHLHE23_E | 334 | CGCGGTTTTGGAGCGT | 265 | CCGAAACGACCGAAAAC | 266 |
| | | TAG | | GAC | |
| C10orf125_B | 335 | CGGTTCGTTGCGTTTA | 267 | CCCCCGAACTACTCTAC | 268 |
| | | TCGA | | GCG | |
| C17orf64_B | 336 | GATTATATTCGGATTTT | 269 | GACTCTTCCTACCCGCG | 270 |
| | | GTTTATCGCGT | | A | |
| CALN1_1520 | 337 | GCGGTTTTTAGTTCGC | 271 | AACAAATAATTAACAAAC | 272 |
| | | GGG | | AACGCCTCC | |
| CALN1_B | 37 | TCGTTCGGCGTATTTA | 273 | CGCGAAAAACTTCCTCC | 274 |
| | | TTTCGTAT | | GA | |
| CD1D_1058 | 339 | GGATTGGTGAGATTCG | 275 | CCCGAAACCAAAAAACA | 276 |
| | | GGAC | | ACGA | |
| CDH4_7890 | 340 | CGGGGAGTTTCGTTTG | 277 | CGAATAACGACTACGAA | 278 |
| | | TATCG | | CTTTAAACG | |
| CHST2_8128 | 341 | CGTAGTTATAGATTTAT | 279 | CTAAAACGATAAAAAAAC | 280 |
| | | TAGAGAGGGCG | | GCGAAACG | |
| CHST2_8384 | 342 | TGGTAGTTTTCGGTAT | 281 | TAACTCTACGCGCAAAA | 282 |
| | | CGACGAG | | CGC | |
| CHST2_9316 | 343 | GGGATTTTTAGCGGAA | 283 | CGACGAACTATCCGACT | 284 |
| | | GCGA | | ATCACT | |
| CHST2_9470 | 344 | CGGAGGAATCGGGTA | 285 | ACTCTCCCATAACAACGA | 286 |
| | | GAATCG | | CTCC | |
| CLIC6_B | 345 | CGCGTAGGGCGAGTTT | 287 | GCCTCCTCCTACCTCTC | 288 |
| | | C | | G | |
| CXCL12_B | 346 | TCGGCGGTTTTTAGTA | 289 | AAATCTCCCGTCCCACT | 290 |
| | | AAAGCG | | CC | |
| DLX4_B | 347 | GGTATATTCGCGTAGG | 291 | AACCGAATACCGAAATCT | 292 |
| | | TGCG | | ATAACCC | |
| DNM3_D | 348 | GTAGTTGGGTTGTAGT | 293 | CCCGAACTTCCCATCGA | 294 |
| | | GCGTG | | AC | |
| EMX1_A | 74 | TTCGTACGGTTTTTTCG | 295 | CCACCACGTAATAATTCT | 296 |
| | | TTTTCG | | TCTCGAAA | |
| ESPN_B | 349 | CGGTTTGATATTATTCG | 297 | AATTAACGCCCCCTATAA | 298 |
| | | GGGTTCG | | CATCC | |
| FAM59B_7764 | 350 | CGCGATAGCGTTTTTT | 299 | CGCACGACCGTAAAATA | 300 |
| | | ATTGTCGCG | | CTCG | |
| FOXP4_B | 351 | CGGTTCGTAGATTGTT | 301 | CAAATACCGTCGAAAAAA | 302 |
| | | TTAGAGCG | | AACTAAATCAAAAC | |
| GP5 | 104 | CGTTGTAGGACGGTTA | 303 | CATCCTACTCTTCGAAAT | 304 |
| | | TGTCG | | AAACCGC | |
| HOXA1_C | 352 | AGTCGTTTTTTTAGGTA | 305 | CGACCTTTACAATCGCC | 306 |
| | | GTTTAGGCG | | GC | |
| IGF2BP3_C | 353 | AGATTGGCGCGTAAAA | 307 | ACCGACCCCGAAAAACG | 308 |
| | | GCG | | | |
| IPTRIPL1_1138 | 354 | CGTTTTCGGAGTCGCG | 309 | AACCATACTTATCCGAAC | 310 |
| | | TG | | GTCTAAAC | |
| IPTRIPL1_1200 | 355 | GAGTAGGGTTATTTTC | 311 | CTACTTTTTTCCCGACAA | 312 |
| | | GCGGG | | AATAAAAACGT | |
| KCNK9_B | 356 | TTTTCGCGTATTTCGTG | 313 | AACGCCGCCGTATTCG | 314 |
| | | GTTC | | | |
| KCNK17_C | 357 | TCGCGTTGGAAGTTGC | 315 | CGTATTCTAAACGCTAAA | 316 |
| | | G | | AAACCGC | |
| KLHDC7B_B | 358 | CGGCGGTAGTTTTGCG | 317 | CTACTAAACAAAAACCAA | 318 |
| | | G | | CACGTCC | |
| LAYN_B | 359 | GGTAGGTTTGTTAGTT | 319 | CGCTATCTCTACGACCG | 320 |
| | | GGTTTTCG | | CCT | |
| LIME1_B | 360 | CGGAGGTAGCGGGCG | 321 | CACTCACCGCTTCCGCC | 322 |
| | | AG | | | |
| LMX1B_D | 361 | GGCGTTCGTTTCGGCG | 323 | CGCTTCTCCGACGCCC | 324 |
| LOC100132891_B | 362 | GCGGTTGAGTTTTTGG | 325 | CCCCGTATAACTAAAAAC | 326 |
| | | TCGG | | GACGAC | |
| MAST1_B | 375 | CGTTTTTTTTATGTAGT | 327 | AAACGACGACGAACGCC | 328 |
| | | AAGCGATTTTTCGC | | | |
| MAX.chr12.427.br | 338 | GCGTTTTGGTTTTTTCG | 329 | GAACGACGAAACTAAAA | 330 |
| | | TTTCGAG | | CCGC | |
| MAX.chr17.73073 | 174 | CGTTTTTTGGTAGTTTT | 331 | GCTTAAACGTAACCGAA | 332 |
| 682-73073814 | | TTTCGAGTCG | | ACGCC | |
| MAX.chr20.4422 | 363 | GGTTGCGCGTCGTTTT | 333 | CCCGACGCGTTTAAATC | 334 |
| | | TTC | | GT | |
| MPZ_5742 | 364 | GGATGGGAATAGTTAA | 335 | TCCAACATTACATACAAC | 336 |
| | | GTTTTAGTCGTT | | ACTAACGTC | |
| MPZ_5554 | 365 | GGTTAGGGGTGGAGTT | 337 | ACTCCGAACTCTACTCAT | 338 |
| | | CGTTA | | CCTTTC | |
| MSX2P1_B | 366 | TAGGTTGGAGATTTTG | 339 | CGAAACCTAAAAACGCC | 340 |
| | | ACGCG | | GAAAC | |
| ODC1_B | 367 | GGTTGGTAGTCGTTTT | 341 | CAAAACCCATCTAATTAC | 342 |
| | | TACGTTTTC | | AAAATACCTCGA | |
| OSR2_A | 234 | TGGAGTTATCGGAAGG | 343 | CGAACTCCCGAAACGAC | 344 |
| | | CGA | | G | |
| OTX1_B | 368 | GGAAATGGTTTAGAGT | 345 | TTCTAAAAAATACTTTCG | 346 |
| | | TTTGGATTTCG | | ATACCGACA | |
| PLXNC1_B | 246 | GTGGTTTGAAGAGTTG | 347 | GCCAAAAATTCGATTCCA | 348 |
| | | TTAGTTCGTTTAG | | ACGCA | |
| PRKCB_7570 | 369 | AAGGTGGGTTGTTTGA | 349 | ACCCTCCGACAAAAAAA | 350 |
| | | AGAAGC | | CGTAC | |
| SCRT2_C | 370 | GCGAGAAGGTTTTGTC | 351 | ACCTACTCACGCACAAC | 352 |
| | | GTAGA | | CT | |
| SLC30A10 | 279 | CGCGGTGAGGAAGAT | 353 | ACGCCACCTACGACTAC | 354 |
| | | CG | | G | |
| SPHK2_B | 371 | GTACGGTTATTGGTTG | 355 | CCGAATCCTCCTCCAAA | 356 |
| | | AGCGG | | CG | |
| ST8SIA4_B | 372 | GGAATTTAATTGGAGA | 357 | CCAAAATTTCCCTCATCT | 358 |
| | | GAAATTTTGGCG | | ATATACGCC | |
| STX16_C | 373 | GTTGCGGGTCGGGTT | 359 | GCAAAAACAAAAAACGC | 360 |
| | | GC | | GTAAAAACC | |
| TBX1_B | 374 | GTCGTCGTTGTCGTAG | 361 | CGTAAAAACCGAACGAC | 362 |
| | | TTGTC | | GCG | |
| TRH_A | 303 | TTTTCGTTGATTTTATT | 363 | GAACCCTCTTCAAATAAA | 364 |
| | | CGAGTCGTC | | CCGC | |
| TRIM67_B | 328 | GATTAAATAGTCGGGG | 365 | ATTCTCCAACGCCAACC | 366 |
| | | TCGCG | | AC | |

TABLE 7

| Gene | DMR | | SEQ |
|---|---|---|---|
| Annotation | No. | Probe Sequence | ID NO: |
| ABLIM1_B | 329 | CGCGCCGAGG CGCGCTTCCACTCC/3C6/ | 367 |
| AJAP1_C | 330 | AGGCCACGGACG | 368 |
| | | GCGGCGTTTTTTTTTATGTTG/3C6/ | |
| ALOX5_B | 331 | AGGCCACGGACG | 369 |
| | | CAACCGAACTAAAAAAAAAAACTAACG/3C6/ | |
| ASCL2_B | 332 | CGCGCCGAGG GCGCGTAAGATTTTCGG/3C6/ | 370 |
| BANK1_B | 333 | CGCGCCGAGG GCGGGTAGTAGTGCG/3C6/ | 371 |
| BHLHE23_E | 334 | CGCGCCGAGG | 372 |
| | | CGACCGAAAAATCGAAAAACA/3C6/ | |
| C10orf125_B | 335 | CGCGCCGAGG | 373 |
| | | GCTAACGCGAATAAAACACG/3C6/ | |
| C17orf64_B | 336 | CGCGCCGAGG | 374 |
| | | TTTTCGTTTTCGGTTTCGG/3C6/ | |
| CALN1_1520 | 337 | CGCGCCGAGG | 375 |
| | | CCGTACCTATTAACTCCG/3C6/ | |
| CALN1_B | 37 | AGGCCACGGACG | 376 |
| | | TCGTTTTTTTTTTGCGGGT/3C6/ | |
| CD1D_1058 | 339 | AGGCCACGGACG | 377 |
| | | CGTATTGGCGCGATTTAG/3C6/ | |
| CDH4_7890 | 340 | AGGCCACGGACG | 378 |
| | | GTTCGAAAAAAACTCGACGAA/3C6/ | |
| CHST2_8128 | 341 | AGGCCACGGACG | 379 |
| | | GCCGTTCTCTAACTTCCG/3C6/ | |
| CHST2_8384 | 342 | AGGCCACGGACG | 380 |
| | | CCGAAATACGAACGCGA/3C6/ | |
| CHST2_9316 | 343 | AGGCCACGGACG | 381 |
| | | TCGTTCCTCGATTTCGC/3C6/ | |
| CHST2_9470 | 344 | AGGCCACGGACG | 382 |
| | | CGAATAAAACCTACGAAAAAAAACG/3C6/ | |
| CLIC6_B | 345 | AGGCCACGGACG | 383 |
| | | GAAAACCGCAAAATCCTCG/3C6/ | |
| CXCL12_B | 346 | AGGCCACGGACG | 384 |
| | | CGCGAAATAAACCTATAATTAACTCA/3C6/ | |
| DLX4_B | 347 | CGCGCCGAGG | 385 |
| | | CCGAACCAACACTCAAAAC/3C6/ | |
| DNM3_D | 348 | CGCGCCGAGG GCGCGTTTGGTTTGGT/3C6/ | 386 |
| EMX1_A | 74 | AGGCCACGGACG AACGCGCTCCAACC/3C6/ | 387 |
| ESPN_B | 349 | CGCGCCGAGG | 388 |
| | | CGCGACGACTAAAAAAATTCA/3C6/ | |
| FAM59B_7764 | 350 | AGGCCACGGACG | 389 |
| | | GTCGAAATCGAAACGCTC/3C6/ | |
| FOXP4_B | 351 | CGCGCCGAGG CCGCGACTACCTCTTC/3C6/ | 390 |
| GP5 | 104 | AGGCCACGGACG | 391 |
| | | CGACGTCCTACAAAACCA/3C6/ | |
| HOXA1_C | 352 | CGCGCCGAGG GGCGGTAGTTGTTGC/3C6/ | 392 |
| IGF2BP3_C | 353 | CGCGCCGAGG GCGAAAACCCCGCC/3C6/ | 393 |
| IPTRIPL1_1138 | 354 | CGCGCCGAGG | 394 |
| | | CGTCTAACTAAACGCGATAAAC/3C6/ | |
| IPTRIPL1_1200 | 355 | CGCGCCGAGG | 395 |
| | | GCGGTTTTAGCGATGAATC/3C6/ | |
| KCNK9_B | 356 | CGCGCCGAGG CGATTCGAGGGCGT/3C6/ | 396 |
| KCNK17_C | 357 | AGGCCACGGACG CGCGACGCAAAACTC/3C6/ | 397 |
| KLHDC7B_B | 358 | AGGCCACGGACG GCGGCGGTTGGATT/3C6/ | 398 |
| LAYN_B | 359 | AGGCCACGGACG | 399 |
| | | TCCCGAAACGAACGATAAA/3C6/ | |
| LIME1_B | 360 | CGCGCCGAGG CGCCGTCGCACTAC/3C6/ | 400 |
| LMX1B_D | 361 | AGGCCACGGACG CGCGACTCCCCACT/3C6/ | 401 |
| LOC100132891_B | 362 | AGGCCACGGACG | 402 |
| | | CGCAAATAATAACGCGAACG/3C6/ | |
| MAST1_B | 375 | AGGCCACGGACG | 403 |
| | | CGTTCGAGGTTAGTTTTTTGG/3C6/ | |
| MAX.chr12.427.br | 338 | AGGCCACGGACG CGTACGTAACCCGCG/3C6/ | 404 |
| MAX.chr17.7307 | 174 | CGCGCCGAGG | 405 |
| 3682-73073814 | | CGCTACTAACCATAACCGC/3C6/ | |
| MAX.chr20.4422 | 363 | CGCGCCGAGG | 406 |
| | | CGTTTTCGTTTGATTCGGTT/3C6/ | |
| MPZ_5742 | 364 | CGCGCCGAGG | 407 |
| | | TCGGTGATTGATGTGTGCG/3C6/ | |
| MPZ_5554 | 365 | CGCGCCGAGG | 408 |
| | | CGTAACTCCATCTCGATAACC/3C6/ | |
| MSX2P1_B | 366 | CGCGCCGAGG CGACCGCGAAAAAACG/3C6/ | 409 |
| ODC1_B | 367 | AGGCCACGGACG | 410 |
| | | CGCGTTGGAAGTTTCG/3C6/ | |
| OSR2_A | 234 | CGCGCCGAGG GCGCGAACACAAAACG/3C6/ | 411 |
| OTX1_B | 368 | CGCGCCGAGG ACCGAAAACGCCCTAAA/3C6/ | 412 |
| PLXNC1_B | 246 | CGCGCCGAGG | 413 |
| | | GCGTGGAGAAATGTTAGTTTG/3C6/ | |
| PRKCB_7570 | 369 | AGGCCACGGACG | 414 |
| | | CGGGCGGTGAATTTGT/3C6/ | |
| SCRT2_C | 370 | AGGCCACGGACG | 415 |
| | | ACGTCGTATTTGTGGCG/3C6/ | |
| SLC30A10 | 279 | AGGCCACGGACG GCGTTGTTTAGCGCG/3C6/ | 416 |
| SPHK2_B | 371 | AGGCCACGGACG | 417 |
| | | GATCCCGCAAATCAACAC/3C6/ | |
| ST8SIA4_B | 372 | CGCGCCGAGG CGATCCCCAACTCCC/3C6/ | 418 |
| STX16_C | 373 | CGCGCCGAGG | 419 |
| | | CGCTTCTAAAACCTCGATCC/3C6/ | |
| TBX1_B | 374 | CGCGCCGAGG | 420 |
| | | CGCGGTCGTTAATATGTATTC/3C6/ | |
| TRH_A | 303 | AGGCCACGGACG | 421 |
| | | CGTTTGGCGTAGATATAAGC/3C6/ | |
| TRIM67_B | 328 | AGGCCACGGACG | 422 |
| | | CGAACTACGAAAACAACCTC/3C6/ | |

TABLE 8

| Marker | DMR | Marker | DMR |
|---|---|---|---|
| AJAP1_C | 330 | CHST2_9316 | 343 |
| C10orf125_B | 335 | ASCL2_B | 332 |
| CALN1_B | 37 | ESPN_B | 349 |
| BHLHE23_E | 334 | DLX4_B | 347 |
| CD1D_1058 | 339 | KCNK17_C | 357 |
| HOXA1_C | 352 | EMX1_A | 74 |
| LOC100132891_B | 362 | MPZ_5742 | 364 |
| MSX2P1_B | 366 | LAYN_B | 359 |
| PRKCB_7570 | 369 | KCNK9_B | 356 |
| ITPRIPL1_1200 | 355 | ABLIM1_B | 329 |
| SPHK2_B | 371 | MAX.chr12.427.br | 338 |
| C17orf64_B | 336 | SCRT2_C | 370 |
| TRIM67_B | 328 | IGF2BP3_C | 353 |
| MAX.chr20.4422 | 363 | MAST1_B | 375 |
| DNM3_D | 348 | MAX.chr17.73073682-73073814 | 174 |
| ODC1_B | 367 | OTX1_B | 368 |
| OSR2_A | 234 | ST8SIA4_B | 372 |
| SLC30A10 | 279 | CXCL12_B | 346 |
| TRH_A | 303 | LIME1_B | 360 |
| ALOX5_B | 331 | TBX1_B | 374 |
| PLXNC1_B | 246 | STX16_C | 373 |
| CDH4_7890 | 340 | FOXP4_B | 351 |
| CLIC6_B | 345 | CALN1_1520 | 337 |
| LMX1B_D | 361 | ITPRIPL1_1138 | 354 |
| FAM59B_7764 | 350 | CHST2_8128 | 341 |
| GP5 | 104 | CHST2_8384 | 342 |
| BANK1_B | 333 | CHST2_9470 | 344 |
| KLHDC7B_B | 358 | MPZ_5554 | 365 |

A collection of 38 normal breast cancer samples were tested for presence of the 56 methylation markers. The 56 markers displayed a range of sensitivities from ˜15% to 92% at 95% specificity. Table 9 shows the markers demonstrating sensitivity at or above 25% at 95% specificity. A 5 marker panel (SPHK2, c17orf64_B, DLX4_B, MPZ_5742, ITPRIPL1_1138) showed 96% sensitivity at 100% specificity. The resulting ROC curve had an AUC of 0.995.

TABLE 9

| Marker | DMR No. | Sensitivity |
|---|---|---|
| AJAP1_C | 330 | 66.30% |
| C10orf125_B | 335 | 58.40% |
| CALN1_B | 37 | 69.70% |
| BHLHE23_E | 334 | 43.80% |
| CD1D_1058 | 339 | 68.50% |
| HOXA1_C | 352 | 62.90% |
| LOC100132891_B | 362 | 79.80% |
| MSX2P1_B | 366 | 79.80% |
| PRKCB_7570 | 369 | 86.50% |
| ITPRIPL1_1200 | 355 | 79.80% |
| SPHK2_B | 371 | 65.20% |
| C17orf64_B | 336 | 77.50% |
| TRIM67_B | 328 | 79.80% |
| MAX.chr20.4422 | 363 | 71.90% |
| CHST2_9316 | 343 | 73.00% |
| ASCL2_B | 332 | 53.90% |
| ESPN_B | 349 | 67.40% |
| DLX4_B | 347 | 83.10% |
| KCNK17_C | 357 | 55.10% |
| EMX1_A | 74 | 77.50% |
| MPZ_5742 | 364 | 91.00% |
| LAYN_B | 359 | 57.30% |
| KCNK9_B | 356 | 62.90% |
| ABLIM1_B | 329 | 44.90% |
| MAX.chr12.427.br | 338 | 79.80% |
| SCRT2_C | 370 | 78.70% |
| IGF2BP3_C | 353 | 70.80% |
| MAST1_B | 375 | 77.50% |
| DNM3_D | 348 | 74.20% |
| ODC1_B | 367 | 65.20% |
| OSR2_A | 234 | 70.80% |
| SLC30A10 | 279 | 60.70% |
| TRH_A | 303 | 85.40% |
| ALOX5_B | 331 | 59.60% |
| PLXNC1_B | 246 | 61.80% |
| CDH4_7890 | 340 | 71.90% |
| CLIC6_B | 345 | 48.30% |
| LMX1B_D | 361 | 56.20% |
| FAM59B_7764 | 350 | 66.30% |
| GP5 | 104 | 61.80% |
| BANK1_B | 333 | 43.80% |
| OTX1_B | 368 | 70.80% |
| ST8SIA4_B | 372 | 40.40% |
| CXCL12_B | 346 | 56.20% |
| LIME1_B | 360 | 47.20% |
| STX16_C | 373 | 52.80% |
| FOXP4_B | 351 | 36.00% |
| CALN1_1520 | 337 | 66.30% |
| ITPRIPL1_1138 | 354 | 83.10% |
| CHST2_8128 | 341 | 62.90% |
| CHST2_8384 | 342 | 60.70% |
| CHST2_9470 | 344 | 66.30% |
| MPZ_5554 | 365 | 92.10% |

Based on the results of the tissue testing, a set of 28 markers were selected to test on a set of plasma samples collected from breast cancer patients and normal controls. The 28 markers were split into two pools of 14 due to the high number of markers to be tested. The markers in the two pools are shown in Tables 10 and 11 below.

TABLE 10
Pool 7 Breast Cancer Plasma Markers

| | |
|---|---|
| AJAP1 | C10orf125 |
| CALN1_B | BHLHE23 |
| LOC100132891 | MSX2P1 |
| SPHK2 | C17orf64 |
| MAST1 | DNM3 |
| MAX.chr.12.427.br | OTX1 |
| SCRT2 | ALOX5 |

TABLE 11
Pool 8 Breast Cancer Plasma Markers

| | |
|---|---|
| FAM59B | ITPRIPL1_B |
| ODC1_B | OSR2_A |
| CD1D_B | DLX4_2591 |
| PRKCB_7570 | MAX.chr20.4422 |
| TRIM67 | MPZ |
| TRH_A | CXCL12_B |
| EMX1_br | CHST2_B |

The testing of Pool 7 markers was done on a collection of EDTA plasma samples comprised of 85 breast cancer samples (33 stage I, 33 stage II, 18 stage III, and 1 stage IV) and 100 healthy normal controls. The testing of Pool 8 markers was done on a similar collection of EDTA plasma samples comprised of 85 breast cancer samples (34 stage I, 32 stage II, 18 stage III and 1 stage IV) and 100 healthy normal controls. Based on the results of the Pool 7 and Pool 8 testing, a collection of 14 assays were selected for further testing (shown in Table 12).

TABLE 12
Pool 9 Breast Cancer Plasma Markers

| | |
|---|---|
| SPHK2 | C17orf64 |
| FAM59B | ITPRIPL1_B |
| ODC1_B | OSR2_A |
| TRIM67 | MPZ |
| TRH_A | CXCL12_B |
| CD1D_B | C10orf125 |
| CALN1_B | CHST2_B |

The testing of Pool 9 markers was done on a collection of LBgard (Biomatrica, San Diego, CA) plasma samples comprised of 42 breast cancer samples (1 stage I, 16 stage II, 14 stage III, and 11 stage IV) and 84 healthy normal controls. Table 13 shows the identified methylated region for the Pool 9 markers. Table 14 shows the exhibited sensitivity and 90% specificity for the Pool 9 markers. Tables 15 and 16 show the primer information, and probe information for the Pool 9 markers. A collection of 4 markers (FAM59B, ITPRIPL1, TRH_A, and C17orf64_B) exhibited a sensitivity of 74% at 90% specificity. The resulting ROC curve exhibited an AUC of 0.884.

TABLE 13

| DMR | | Region on Chromosome |
|---|---|---|
| No. | Gene Annotation | (starting base-ending base) |
| 47 | CD1D | chr1: 158150864-158151129 |
| 134 | ITPRIPL1 | chr2: 96990968-96991328 |
| 90 | FAM59B | chr2: 26407713-26407972 |
| 27 | C10orf125 | chr10: 135171410-135171504 |
| 305 | TRIM67 | chr1: 231297047-231297159 |
| 284 | SPHK2 | chr19: 49127580-49127683 |
| 37 | CALN1_B | chr7: 71801741-71801800 |
| 57 | CHST2_B | chr3: 142839223-142839568 |
| 221 | MPZ | chr1: 161275561-161275996 |
| 346 | CXCL12_B | chr10: 44881200-44881315 |
| 367 | ODC1_B | chr2: 10589075-10589225 |
| 234 | OSR2_A | chr8: 99952233-99952366 |
| 303 | TRH_A | chr3: 129693484-129693575 |
| 336 | C17orf64_B | chr17: 58499085-58499196 |

TABLE 14

| Marker | | Sens @ | DMR |
|---|---|---|---|
| Name | AUC | 90% sp | No. |
| FAM59B | 0.814 | 50.0% | 90 |
| ITPRIPL1 | 0.804 | 61.9% | 134 |
| ODC1_B | 0.809 | 59.5% | 367 |
| OSR2_A | 0.749 | 42.9% | 234 |
| TRIM67 | 0.669 | 30.9% | 305 |
| MPZ | 0.698 | 47.6% | 221 |
| TRH_A | 0.83 | 50.0% | 303 |
| CXCL12_B | 0.71 | 28.6% | 346 |
| SPHK2 | 0.585 | 31.0% | 284 |
| C17orf64_B | 0.763 | 59.5% | 336 |
| CD1D | 0.613 | 33.3% | 47 |
| C10orf125 | 0.775 | 45.2% | 27 |
| CALN1_B | 0.622 | 26.2% | 37 |
| CHST2_B | 0.687 | 38.1% | 57 |

TABLE 15

| Gene | DMR | Forward | SEQ ID | Reverse Primer | SEQ ID |
|---|---|---|---|---|---|
| Annotation | No. | Primer 5′-3′ | NO: | 5′-3′ | NO: |
| CD1D | 47 | GGATTGGTGA | 423 | CCCGAAACCAAA | 424 |
| | | GATTCGGGAC | | AAACAACGA | |
| ITPRIPL1 | 134 | GAGTAGGGTT | 425 | CTACTTTTTTCC | 426 |
| | | ATTTTCGCGG | | CGACAAAATAAA | |
| | | G | | AACGT | |
| FAM59B | 90 | CGCGATAGCG | 427 | CGCACGACCGT | 428 |
| | | TTTTTTATTGT | | AAAATACTCG | |
| | | CGCG | | | |
| C10orf125 | 27 | CGGTTCGTTG | 429 | CCCCCGAACTAC | 430 |
| | | CGTTTATCGA | | TCTACGCG | |
| TRIM67 | 305 | GATTAAATAGT | 431 | ATTCTCCAACGC | 432 |
| | | CGGGGTCGC | | CAACCAC | |
| | | G | | | |
| SPHK2 | 284 | GTACGGTTAT | 433 | CCGAATCCTCCT | 434 |
| | | TGGTTGAGCG | | CCAAACG | |
| | | G | | | |
| CALN1_B | 37 | TCGTTCGGCG | 273 | CGCGAAAAACTT | 274 |
| | | TATTTATTTCG | | CCTCCGA | |
| | | TAT | | | |
| CHST2_B | 57 | GGGATTTTTA | 437 | CGACGAACTATC | 438 |
| | | GCGGAAGCGA | | CGACTATCACT | |
| MPZ | 221 | GGTTAGGGGT | 439 | ACTCCGAACTCT | 440 |
| | | GGAGTTCGTT | | ACTCATCCTTTC | |
| | | A | | | |
| CXCL12_B | 346 | TCGGCGGTTT | 441 | AAATCTCCCGTC | 442 |
| | | TTAGTAAAAG | | CCACTCC | |
| | | CG | | | |
| ODC1_B | 367 | GGTTGGTAGT | 443 | CAAAACCCATCT | 444 |
| | | CGTTTTTACGT | | AATTACAAAATA | |
| | | TTTC | | CCTCGA | |
| OSR2_A | 234 | TGGAGTTATC | 445 | CGAACTCCCGAA | 446 |
| | | GGAAGGCGA | | ACGACG | |
| TRH_A | 303 | TTTTCGTTGAT | 447 | GAACCCTCTTCA | 448 |
| | | TTTATTCGAGT | | AATAAACCGC | |
| | | CGTC | | | |
| C17orf64_B | 336 | GATTATATTCG | 449 | GACTCTTCCTAC | 450 |
| | | GATTTTGTTTA | | CCGCGA | |
| | | TCGCGT | | | |

TABLE 16

| Gene | DMR | | SEQ ID |
|---|---|---|---|
| Annotation | No. | Probe Sequence | NO: |
| CD1D | 47 | AGGCCACGGACG | 451 |
| | | CGTATTGGCGCGATTTAG/3C6/ | |
| ITPRIPL1 | 134 | CGCGCCGAGG | 452 |
| | | GCGGTTTTAGCGATGAATC/3C6/ | |
| FAM59B | 90 | AGGCCACGGACG | 453 |
| | | GTCGAAATCGAAACGCTC/3C6/ | |
| C10orf125 | 27 | CGCGCCGAGG | 454 |
| | | GCTAACGCGAATAAAACACG/3C6/ | |
| TRIM67 | 305 | AGGCCACGGACG | 455 |
| | | CGAACTACGAAAACAACCTC/3C6/ | |
| SPHK2 | 284 | AGGCCACGGACG | 456 |
| | | GATCCCGCAAATCAACAC/3C6/ | |
| CALN1_B | 37 | AGGCCACGGACG | 376 |
| | | TCGTTTTTTTTTTGCGGGT/3C6/ | |
| CHST2_B | 57 | CGCGCCGAGG | 458 |
| | | TCGTTCCTCGATTTCGC/3C6/ | |
| MPZ | 221 | CGCGCCGAGG | 459 |
| | | CGTAACTCCATCTCGATAACC/3C6/ | |
| CXCL12_B | 346 | CGCGCCGAGG | 460 |
| | | CGCGAAATAAACCTATAATTAACTCA/3C6/ | |
| ODC1_B | 367 | AGGCCACGGACG | 461 |
| | | CGCGTTGGAAGTTTCG/3C6/ | |
| OSR2_A | 234 | CGCGCCGAGG | 462 |
| | | GCGCGAACACAAAACG/3C6/ | |
| TRH_A | 303 | AGGCCACGGACG | 463 |
| | | CGTTTGGCGTAGATATAAGC/3C6/ | |
| C17orf64_B | 336 | CGCGCCGAGG | 464 |
| | | TTTTCGTTTTCGGTTTCGG/3C6/ | |

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1-20. (canceled)

21. A method comprising:
treating DNA in a sample from a subject with a reagent that modifies DNA in a methylation-specific manner;
amplifying the treated DNA using a set of primers for one or more genes selected from TRH, BANK1, C10orf125, C17orf64, CALN1, CD1D, CHST2, FAIM2, FAM59B, ITPRIPL1, MPZ, ODC1, OSR2, OTX1, RYR2, SPHK2, and/or TRIM67; and
determining a methylation level of the one or more genes using polymerase chain reaction, nucleic acid sequencing, mass spectrometry, methylation-specific nuclease, mass-based separation, and/or target capture.

22. The method of claim 21, wherein the reagent that modifies DNA in a methylation-specific manner comprises a methylation-sensitive restriction enzyme, a methylation-dependent restriction enzyme, and/or a bisulfite reagent.

23. The method of claim 21, wherein the reagent that modifies DNA in a methylation-specific manner is a bisulfite reagent, and wherein treatment with the bisulfite reagent produces bisulfite-treated DNA.

24. The method of claim 21, wherein determining the methylation level of each of the one or more genes comprises multiplex amplification.

25. The method of claim 21, wherein determining the methylation level of the one or more genes comprises using multiplex amplification, methylation-specific PCR, quantitative methylation-specific PCR, methylation-specific DNA restriction enzyme analysis, quantitative bisulfite pyrosequencing, flap endonuclease assay, PCR-flap assay, and/or bisulfite genomic sequencing PCR.

26. The method of claim 21, wherein the sample is a blood sample, a serum sample, a plasma sample, or a tissue sample.

27. The method of claim 26, wherein the tissue sample is a breast tissue sample.

28. The method of claim 21, wherein determining the methylation level of the one or more genes comprises determining a methylation score for one or more CpG sites and/or determining a methylation frequency for one or more CpG sites.

29. The method of claim 28, wherein the one or more CpG sites are present in a coding region or a regulatory region.

30. The method of claim 21, wherein the method further comprises determining a methylation level of one or more corresponding genes from a control sample.

31. The method of claim 30, wherein the control sample is from a subject that does not have breast cancer.

32. The method of claim 30, wherein comparing the methylation level of the one or more genes to the methylation level of the one or more corresponding genes from the control sample indicates that the subject has breast cancer or has an increased risk of developing breast cancer.

32. The method of claim 30, wherein the methylation level of the one or more genes is increased compared to the methylation level of the one or more corresponding genes from a control sample.

33. The method of claim 21, wherein:
the set of primers specific for TRH comprises SEQ ID NOs: 245 and 246;
the set of primers specific for BANK1 comprise SEQ ID NOs: 423 and 424;
the set of primers specific for C10orf125 comprises SEQ ID NOs: 429 and 430;
the set of primers specific C17orf64 comprises SEQ ID NOs: 269 and 270, or SEQ ID NOs: 449 and 450;
the set of primers specific for CALN1 comprises SEQ ID NOs: 273 and 274;
the set of primers specific for CD1D comprises SEQ ID NOs: 33 and 34;
the set of primers specific for CHST2 comprises SEQ ID NOs: 39 and 40;
the set of primers specific for FAM59B comprises SEQ ID NOs: 245 and 246, or SEQ ID NOs: 427 and 428;
the set of primers specific for ITPRIPL1 comprises SEQ ID NOs: 97 and 98, SEQ ID NOs: 99 and 100, or SEQ ID NOs: 425 and 426;
the set of primers specific for MPZ comprises SEQ ID NOs: 175 and 176, or SEQ ID NOs: 439 and 440;
the set of primers specific for ODC1 comprises SEQ ID NOs: 341 and 342, or SEQ ID NOs: 443 and 444;
the set of primers specific for OSR2 comprises SEQ ID NOs: 187 and 188;
the set of primers specific OTX1 comprises SEQ ID NOs: 289 and 290, or SEQ ID NOs: 441 and 442;
the set of primers specific for SPHK2 comprises SEQ ID NOs: 433 and 434; and
the set of primers specific for TRIM67 comprises SEQ ID NOs: 431 and 432.

34. The method of claim 21, wherein the one or more genes comprises TRH.
